SEQUENCE LISTING (30 sequences)

SEQ ID NO: 1 - protein, 20 aa, Artificial Sequence
  Description of Artificial Sequence Synthetic peptide
RRANAALKAGELYKSILYGC

SEQ ID NO: 2 - protein, 11 aa, Artificial Sequence
  Description of Artificial Sequence Synthetic peptide
RLDGNEIKRGC

SEQ ID NO: 3 - protein, 15 aa, Artificial Sequence
  Description of Artificial Sequence Synthetic peptide
AHEEISTTNEGVMGC

SEQ ID NO: 4 - protein, 30 aa, Artificial Sequence
  Description of Artificial Sequence Synthetic polypeptide
NGVFKYRPRYFLYKHAYFYPPLKRFPVQGC

SEQ ID NO: 5 - protein, 10 aa, Artificial Sequence
  Description of Artificial Sequence Synthetic peptide
CQDSETRTFY

SEQ ID NO: 6 - protein, 9 aa, Artificial Sequence
  Description of Artificial Sequence Synthetic peptide
TKKTLRTGC

SEQ ID NO: 7 - protein, 30 aa, Artificial Sequence
  Description of Artificial Sequence Synthetic polypeptide
GLRSKSKKFRRPDIQYPDATDEDITSHMGC

SEQ ID NO: 8 - protein, 9 aa, Artificial Sequence
  Description of Artificial Sequence Synthetic peptide
SQNPVQPGC

SEQ ID NO: 9 - protein, 13 aa, Artificial Sequence
  Description of Artificial Sequence Synthetic peptide
SYIRIADTNITGC

SEQ ID NO: 10 - protein, 11 aa, Artificial Sequence
  Description of Artificial Sequence Synthetic peptide
SYIRIADTNIT

SEQ ID NO: 11 - protein, 8 aa, Artificial Sequence
  Description of Artificial Sequence Synthetic peptide
KELNLVYT

SEQ ID NO: 12 - protein, 10 aa, Artificial Sequence
  Description of Artificial Sequence Synthetic peptide
KELNLVYTGC

SEQ ID NO: 13 - protein, 11 aa, Artificial Sequence
  Description of Artificial Sequence Synthetic peptide
GELYKSILYGC

SEQ ID NO: 14 - protein, 12 aa, Artificial Sequence
  Description of Artificial Sequence Synthetic peptide
GSITTIDVPWNV

SEQ ID NO: 15 - protein, 12 aa, Artificial Sequence
  Description of Artificial Sequence Synthetic peptide
GCGGELYKSILY

SEQ ID NO: 16 - protein, 14 aa, Artificial Sequence
  Description of Artificial Sequence Synthetic peptide
GSITTIDVPWNVGC

SEQ ID NO: 17 - protein, 18 aa, Artificial Sequence
  Description of Artificial Sequence Synthetic peptide
RRANAALKAGELYKSILY

SEQ ID NO: 18 - protein, 9 aa, Artificial Sequence
  Description of Artificial Sequence Synthetic peptide
RLDGNEIKR

SEQ ID NO: 19 - protein, 13 aa, Artificial Sequence
  Description of Artificial Sequence Synthetic peptide
AHEEISTTNEGVM

SEQ ID NO: 20 - protein, 28 aa, Artificial Sequence
  Description of Artificial Sequence Synthetic peptide
NGVFKYRPRYFLYKHAYFYPPLKRFPVQ

SEQ ID NO: 21 - protein, 7 aa, Artificial Sequence
  Description of Artificial Sequence Synthetic peptide
TKKTLRT

SEQ ID NO: 22 - protein, 28 aa, Artificial Sequence
  Description of Artificial Sequence Synthetic peptide
GLRSKSKKFRRPDIQYPDATDEDITSHM

SEQ ID NO: 23 - protein, 7 aa, Artificial Sequence
  Description of Artificial Sequence Synthetic peptide
SQNPVQP

SEQ ID NO: 24 - protein, 11 aa, Artificial Sequence
  Description of Artificial Sequence Synthetic peptide
SYIRIADTNIT

SEQ ID NO: 25 - protein, 9 aa, Artificial Sequence
  Description of Artificial Sequence Synthetic peptide
GELYKSILY

SEQ ID NO: 26 - protein, 12 aa, Artificial Sequence
  Description of Artificial Sequence Synthetic peptide
CQDSETRTFYGC

SEQ ID NO: 27 - protein, 14 aa, Artificial Sequence
  Description of Artificial Sequence Synthetic peptide
GCGGELYKSILYGC

SEQ ID NO: 28 - protein, 21 aa, Artificial Sequence
  Description of Artificial Sequence Synthetic peptide
GRRANAALKAGELYKSILYGC

SEQ ID NO: 29 - protein, 12 aa, Artificial Sequence
  Description of Artificial Sequence Synthetic peptide
GSYIRIADTNIT

SEQ ID NO: 30 - protein, 14 aa, Artificial Sequence
  Description of Artificial Sequence Synthetic peptide
GSYIRIADTNITGC

(19) United States
(12) Patent Application Publication
(10) Pub. No.: US 2016/0229895 A1
(43) Pub. Date: Aug. 11, 2016

(54) COLLAGEN-BINDING SYNTHETIC PEPTIDOGLYCANS FOR USE IN VASCULAR INTERVENTION

(71) Applicant: Symic IP, LLC., San Francisco, CA (US)

(72) Inventors: John Eric Paderi, San Francisco, CA (US); Alyssa Panitch, West Lafayette, IN (US); Kinam Park, West Lafayette, CA (US); Katherine Allison Stuart, West Lafayette, IN (US)

(73) Assignee: Symic IP, LLC., San Francisco, CA (US)

(21) Appl. No.: 15/042,684

(22) Filed: Feb. 12, 2016

Related U.S. Application Data
(63) Continuation of application No. 14/474,832, filed on Sep. 2, 2014, now abandoned.
Application No. 14/474,832 is a continuation of application No. 13/806,438, filed on Dec. 21, 2012, now abandoned.
(60) Provisional application No. 61/357,912, filed on Jun. 23, 2010.

(51) Int. Cl. C07K 7/08

(52) U.S. Cl. CPC C07K 7/08; A61K 38/00

(57) ABSTRACT

This invention relates to collagen-binding synthetic peptidoglycans. More particularly, this invention relates to collagen-binding synthetic peptidoglycans for use in vascular intervention procedures. The invention also relates to kits comprising such collagen-binding synthetic peptidoglycans and catheters or stents.

### CROSS REFERENCE TO RELATED APPLICATIONS

This application is a continuation of U.S. patent application Ser. No. 14/474,832, filed Sep. 2, 2014, which is a continuation of U.S. patent application Ser. No. 13/806,438, filed Dec. 21, 2012, which is a U.S. National Phase Application of PCT/US2011/041654, filed Jun. 23, 2011, which claims priority under 35 U.S.C. §119(e) to U.S. Provisional Application No. 61/357,912, filed on Jun. 23, 2010, the entire disclosures of which are incorporated herein by reference.

### SEQUENCE LISTING

The instant application contains a Sequence Listing which has been submitted in U.S. patent application Ser. No. 13/806,438, filed Dec. 21, 2012, in ASCII format via EFS-Web and is hereby incorporated by reference in its entirety. Said ASCII copy, created on Mar. 15, 2013, is named 322022US.txt and is 8,695 bytes in size.

### TECHNICAL FIELD

This invention pertains to the field of collagen-binding synthetic peptidoglycans. More particularly, this invention relates to collagen-binding synthetic peptidoglycans for use in vascular intervention procedures.

### BACKGROUND AND SUMMARY OF THE INVENTION

Vascular interventions that involve a medical device inserted or implanted into the body of a patient, for example, angioplasty, stenting, atherectomy and grafting, are often associated with undesirable effects. For example, the insertion or implantation of catheters or stents can lead to the formation of emboli or clots in blood vessels. Other adverse reactions to vascular intervention can include hyperplasia, restenosis, occlusion of blood vessels, platelet aggregation, and calcification.

The number of percutaneous coronary intervention (PCI) procedures, commonly known as balloon angioplasty, has increased by 30% over the past 10 years totaling more than 1.3 million patients in the U.S. annually at a cost of more than $60 billion. During percutaneous coronary intervention, guide catheters are advanced from the periphery, usually the femoral artery, into the aorta. The tip of the catheter is positioned in the ostium of a coronary artery. Subsequently, wires, balloon catheters, or other devices are advanced through the guide catheter into the large epicardial coronary arteries to treat stenotic lesions.

The large increase in the number of procedures is due to a rise in heart disease as well as technological advancements, which has led to safer and more effective practice. Still, PCI procedures are not without problems including thrombosis and intimal hyperplasia, which are complications from the procedure. Areas of focus for mitigating these complications are the coagulation and inflammatory responses which occur at the vessel wall as a result of the procedure. Balloon inflation results in endothelial denudation of the vessel wall, which initiates coagulation and inflammation through platelet activation and is currently a possible consequence of PCI procedures.

The synthetic collagen-binding peptidoglycans described herein can be synthesized with design control and in large quantities at low cost, making their clinical use feasible. As described herein the synthetic collagen-binding peptidoglycans are designed to bind collagen with high affinity, where they remain bound during blood flow to prevent platelet binding to exposed collagen of the denuded endothelium and, consequently, to prevent platelet activation, thrombosis, inflammation resulting from denuding the endothelium, intimal hyperplasia, and vasospasm. The collagen-binding synthetic peptidoglycans described herein can also stimulate endothelial cell proliferation and can bind to collagen in a denuded vessel.

The following numbered embodiments are contemplated and are non-limiting:

1. A method for vascular intervention, said method comprising the steps of providing a collagen-binding synthetic peptidoglycan; and administering the collagen-binding synthetic peptidoglycan to a patient, wherein the collagen-binding synthetic peptidoglycan is administered to the patient prior to during, or after the vascular intervention and binds to a denuded vessel in the patient.

2. The method of clause 1 wherein the collagen-binding synthetic peptidoglycan inhibits platelet activation.

3. The method of any one of clauses 1 to 2 wherein the collagen-binding synthetic peptidoglycan inhibits platelet binding to the denuded vessel.

4. The method of any one of clauses 1 to 3 wherein the collagen-binding synthetic peptidoglycan inhibits intimal hyperplasia.

5. The method of any one of clauses 1 to 4 wherein the collagen-binding synthetic peptidoglycan inhibits inflammation resulting from denuding of the vessel.

6. The method of any one of clauses 1 to 5 wherein the collagen-binding synthetic peptidoglycan inhibits thrombosis.

7. The method of any one of clauses 1 to 6 wherein the collagen-binding synthetic peptidoglycan inhibits vasospasm.

8. The method of any one of clauses 1 to 7 wherein the collagen-binding synthetic peptidoglycan stimulates endothelial cell proliferation.

9. The method of any one of clauses 1 to 8 wherein the collagen-binding synthetic peptidoglycan binds to exposed collagen on the denuded vessel.

10. The method of any one of clauses 1 to 9 wherein the collagen-binding synthetic peptidoglycan is a compound of formula

PₙGₓ wherein n is 1 to 50;

x is 1 to 10

P is a synthetic peptide of about 5 to about 40 amino acids comprising a sequence of a collagen-binding domain; and

G is a glycan.

11. The method of any one of clauses 1 to 9 wherein the collagen-binding synthetic peptidoglycan is a compound of formula

(PₙL)ₓG wherein n is 1 to 7;

x is 1 to 50;

P is a synthetic peptide of about 5 to about 40 amino acids comprising a sequence of a collagen-binding domain;

L is a linker; and

G is a glycan.

12. The method of any one of clauses 1 to 9 wherein the collagen-binding synthetic peptidoglycan is a compound of formula

P(LGₙ)ₓ wherein n is 1 to 5;

x is 1 to 10;

P is a synthetic peptide of about 5 to about 40 amino acids comprising a sequence of a collagen-binding domain;

L is a linker; and

G is a glycan.

13. The method of any one of clauses 1 to 9 wherein the collagen-binding synthetic peptidoglycan is a compound of formula

PₙGₓ wherein n is MWG/1000;

wherein MWG is the molecular weight of G rounded to the nearest 1 kDa;

wherein x is 1 to 10;

wherein P is a synthetic peptide of about 5 to about 40 amino acids comprising a sequence of a collagen-binding domain; and

wherein G is a glycan.

14. The method of any one of clauses 1 to 9 wherein the collagen-binding synthetic peptidoglycan is a compound of formula

(PₙL)ₓG wherein n is 1 to 7;

wherein x is MWG/1000;

wherein MWG is the molecular weight of G rounded to the nearest 1 kDa;

wherein P is a synthetic peptide of about 5 to about 40 amino acids comprising a sequence of a collagen-binding domain;

wherein L is a linker; and

wherein G is a glycan.

15. The method of any one of clauses 1 to 14 wherein the glycan is a glycosaminoglycan or a polysaccharide.

16. The method of any one of clauses 1 to 15 wherein the glycan component of the peptidoglycan is selected from the group consisting of alginate, agarose, dextran, chondroitin, dermatan, dermatan sulfate, heparan, heparin, keratin, and hyaluronan.

17. The method of any one of clauses 1 to 16 wherein the glycan component of the peptidoglycan is selected from the group consisting of dermatan sulfate, dextran, hyaluronan, and heparin.

18. The method of any one of clauses 1 to 17 wherein the glycan is dermatan sulfate.

19. The method of any one of clauses 1 to 18 wherein the peptide component of the peptidoglycan comprises an amino acid sequence selected from the group consisting of RRANAALKAGELYKSILYGC (SEQ ID NO: 1), RLDGNEIKRGC (SEQ ID NO: 2), AHEEISTTNEGVMGC (SEQ ID NO: 3), NGVFKYRPRYFLYKHAYFYPPLKRFPVQGC (SEQ ID NO: 4), CQDSETRTFY (SEQ ID NO: 5), TKKTLRTGC (SEQ ID NO: 6), GLRSKSKKFRRPDIQYPDATDEDITSHMGC (SEQ ID NO: 7), SQNPVQPGC (SEQ ID NO: 8), SYIRIADTNITGC (SEQ ID NO: 9), SYIRIADTNIT (SEQ ID NO: 10), KELNLVYT (SEQ ID NO: 11), KELNLVYTGC (SEQ ID NO: 12), GELYKSILYGC (SEQ ID NO: 13), GSITTIDVPWNV (SEQ ID NO: 14), GCGGELYKSILY (SEQ ID NO: 15) and GSITTIDVPWNVGC (SEQ ID NO: 16).

20. The method of any one of clauses 1 to 19 wherein the peptide component of the peptidoglycan comprises an amino acid sequence of RRANAALKAGELYKSILYGC (SEQ ID NO: 1).

21. The method of any one of clauses 1 to 20 wherein the collagen-binding synthetic peptidoglycan is DS-SILY₁₈.

22. The method of any one of clauses 1 to 21 wherein the collagen-binding synthetic peptidoglycan is administered to the patient parenterally.

23. The method of clause 22 wherein the parenteral administration is through a route selected from the group consisting of intravascular, intravenous, intraarterial, intramuscular, cutaneous, subcutaneous, percutaneous, intradermal, and intraepidermal.

24. The method of clause 22 or 23 wherein the collagen-binding synthetic peptidoglycan is administered parenterally using a needle or a device for infusion.

25. The method of any one of clauses 1 to 24 wherein the collagen-binding synthetic peptidoglycan is administered to the patient with a catheter, as a coating on a balloon, through a porous balloon, or as a coating on a stent.

26. A compound for use in vascular intervention in a patient, said compound comprising a collagen-binding synthetic peptidoglycan wherein the collagen-binding synthetic peptidoglycan binds to a denuded vessel in the patient.

27. The compound of clause 26 wherein the collagen-binding synthetic peptidoglycan inhibits platelet activation.

28. The compound of any one of clauses 26 to 27 wherein the collagen-binding synthetic peptidoglycan inhibits platelet binding to the denuded vessel.

29. The compound of any one of clauses 26 to 28 wherein the collagen-binding synthetic peptidoglycan inhibits intimal hyperplasia.

30. The compound of any one of clauses 26 to 29 wherein the collagen-binding synthetic peptidoglycan inhibits inflammation resulting from denuding of the vessel.

31. The compound of any one of clauses 26 to 30 wherein the collagen-binding synthetic peptidoglycan inhibits thrombosis.

32. The compound of any one of clauses 26 to 31 wherein the collagen-binding synthetic peptidoglycan inhibits vasospasm.

33. The compound of any one of clauses 26 to 32 wherein the collagen-binding synthetic peptidoglycan stimulates endothelial cell proliferation.

34. The compound of any one of clauses 26 to 33 wherein the collagen-binding synthetic peptidoglycan binds to exposed collagen on the denuded vessel.

35. The compound of any one of clauses 26 to 34 wherein the collagen-binding synthetic peptidoglycan is a compound of formula

PₙGₓ wherein n is 1 to 50;

x is 1 to 10

P is a synthetic peptide of about 5 to about 40 amino acids comprising a sequence of a collagen-binding domain; and

G is a glycan.

36. The compound of any one of clauses 26 to 34 wherein the collagen-binding synthetic peptidoglycan is a compound of formula

(PₙL)ₓG wherein n is 1 to 7;

x is 1 to 10;

P is a synthetic peptide of about 5 to about 40 amino acids comprising a sequence of a collagen-binding domain;

L is a linker; and

G is a glycan.

37. The compound of any one of clauses 26 to 34 wherein the collagen-binding synthetic peptidoglycan is a compound of formula

P(LGₙ)ₓ wherein n is 1 to 5;

x is 1 to 10;

P is a synthetic peptide of about 5 to about 40 amino acids comprising a sequence of a collagen-binding domain;

L is a linker; and

G is a glycan.

38. The compound of any one of clauses 26 to 34 wherein the collagen-binding synthetic peptidoglycan is a compound of formula

PₙGₓ wherein n is MWG/1000;

wherein MWG is the molecular weight of G rounded to the nearest 1 kDa;

wherein x is 1 to 10;

wherein P is a synthetic peptide of about 5 to about 40 amino acids comprising a sequence of a collagen-binding domain; and

wherein G is a glycan.

39. The compound of any one of clauses 26 to 34 wherein the collagen-binding synthetic peptidoglycan is a compound of formula

(PₙL)ₓG wherein n is 1 to 7;

wherein x is MWG/1000;

wherein MWG is the molecular weight of G rounded to the nearest 1 kDa;

wherein P is a synthetic peptide of about 5 to about 40 amino acids comprising a sequence of a collagen-binding domain;

wherein L is a linker; and

wherein G is a glycan.

40. The compound of any one of clauses 26 to 39 wherein the glycan is a glycosaminoglycan or a polysaccharide.

41. The compound of any one of clauses 26 to 40 wherein the glycan component of the peptidoglycan is selected from the group consisting of alginate, agarose, dextran, chondroitin, dermatan, dermatan sulfate, heparan, heparin, keratin, and hyaluronan.

42. The compound of any one of clauses 26 to 41 wherein the glycan component of the peptidoglycan is selected from the group consisting of dermatan sulfate, dextran, hyaluronan, and heparin.

43. The compound of any one of clauses 26 to 42 wherein the glycan is dermatan sulfate.

44. The compound of any one of clauses 26 to 43 wherein the peptide component of the peptidoglycan comprises an amino acid sequence selected from the group consisting of RRANAALKAGELYKSILYGC (SEQ ID NO: 1), RLDGNEIKRGC (SEQ ID NO: 2), AHEEISTTNEGVMGC (SEQ ID NO: 3), NGVFKYRPRYFLYKHAYFYPPLKRFPVQGC (SEQ ID NO: 4), CQDSETRTFY (SEQ ID NO: 5), TKKTLRTGC (SEQ ID NO: 6), GLRSKSKKFRRPDIQYPDATDEDITSHMGC (SEQ ID NO: 7), SQNPVQPGC (SEQ ID NO: 8), SYIRIADTNITGC (SEQ ID NO: 9), SYIRIADTNIT (SEQ ID NO: 10), KELNLVYT (SEQ ID NO: 11), KELNLVYTGC (SEQ ID NO: 12), GELYKSILYGC (SEQ ID NO: 13), GSITTIDVPWNV (SEQ ID NO: 14), GCGGELYKSILY (SEQ ID NO: 15) and GSITTIDVPWNVGC (SEQ ID NO: 16).

45. The compound of any one of clauses 26 to 44 wherein the peptide component of the peptidoglycan comprises an amino acid sequence of RRANAALKAGELYKSILYGC (SEQ ID NO: 1).

46. The compound of any one of clauses 26 to 45 wherein the collagen-binding synthetic peptidoglycan is DS-SILY₁₈.

47. The compound of any one of clauses 26 to 46 wherein the collagen-binding synthetic peptidoglycan is administered to the patient parenterally.

48. The compound of clause 47 wherein the parenteral administration is through a route selected from the group consisting of intravascular, intravenous, intraarterial, intramuscular, cutaneous, subcutaneous, percutaneous, intradermal, and intraepidermal.

49. The compound of clause 47 or 48 wherein the collagen-binding synthetic peptidoglycan is administered parenterally using a needle or a device for infusion.

50. The compound of any one of clauses 26 to 49 wherein the collagen-binding synthetic peptidoglycan is administered to the patient with a catheter, as a coating on a balloon, through a porous balloon, or as a coating on a stent.

51. A kit comprising

a collagen-binding synthetic peptidoglycan; and

a component selected from the group consisting of a catheter, a stent, a balloon, and a combination thereof.

52. The kit of clause 51 wherein the collagen-binding synthetic peptidoglycan is a compound of formula

PₙGₓ wherein n is 1 to 50;

x is 1 to 10

P is a synthetic peptide of about 5 to about 40 amino acids comprising a sequence of a collagen-binding domain; and

G is a glycan.

53. The kit of clause 51 wherein the collagen-binding synthetic peptidoglycan is a compound of formula

(PₙL)ₓG wherein n is 1 to 7;

x is 1 to 10;

P is a synthetic peptide of about 5 to about 40 amino acids comprising a sequence of a collagen-binding domain;

L is a linker; and

G is a glycan.

54. The kit of clause 51 wherein the collagen-binding synthetic peptidoglycan is a compound of formula

P(LGₙ)ₓ wherein n is 1 to 5;

x is 1 to 10;

P is a synthetic peptide of about 5 to about 40 amino acids comprising a sequence of a collagen-binding domain;

L is a linker; and

G is a glycan.

55. The kit of clause 51 wherein the collagen-binding synthetic peptidoglycan is a compound of formula

PₙGₓ wherein n is MWG/1000;

wherein MWG is the molecular weight of G rounded to the nearest 1 kDa;

wherein x is 1 to 10;

wherein P is a synthetic peptide of about 5 to about 40 amino acids comprising a sequence of a collagen-binding domain; and

wherein G is a glycan.

56. The kit of clause 51 wherein the collagen-binding synthetic peptidoglycan is a compound of formula

(PₙL)ₓG wherein n is 1 to 7;

wherein x is MWG/1000;

wherein MWG is the molecular weight of G rounded to the nearest 1 kDa;

wherein P is a synthetic peptide of about 5 to about 40 amino acids comprising a sequence of a collagen-binding domain;

wherein L is a linker; and

wherein G is a glycan.

57. The kit of any one of clauses 51 to 56 wherein the glycan is a glycosaminoglycan or a polysaccharide.

58. The kit of any one of clauses 51 to 57 wherein the glycan component of the peptidoglycan is selected from the group consisting of alginate, agarose, dextran, chondroitin, dermatan, dermatan sulfate, heparan, heparin, keratin, and hyaluronan.

59. The kit of any one of clauses 51 to 58 wherein the glycan component of the peptidoglycan is selected from the group consisting of dermatan sulfate, dextran, hyaluronan, and heparin.

60. The kit of any one of clauses 51 to 59 wherein the glycan is dermatan sulfate.

61. The kit of any one of clauses 51 to 60 wherein the peptide component of the peptidoglycan comprises an amino acid sequence selected from the group consisting of RRANAALKAGELYKSILYGC (SEQ ID NO: 1), RLDGNEIKRGC (SEQ ID NO: 2), AHEEISTTNEGVMGC (SEQ ID NO: 3), NGVFKYRPRYFLYKHAYFYPPLKRFPVQGC (SEQ ID NO: 4), CQDSETRTFY (SEQ ID NO: 5), TKKTLRTGC (SEQ ID NO: 6), GLRSKSKKFRRPDIQYPDATDEDITSHMGC (SEQ ID NO: 7), SQNPVQPGC (SEQ ID NO: 8), SYIRIADTNITGC (SEQ ID NO: 9), SYIRIADTNIT (SEQ ID NO: 10), KELNLVYT (SEQ ID NO: 11), KELNLVYTGC (SEQ ID NO: 12), GELYKSILYGC (SEQ ID NO: 13), GSITTIDVPWNV (SEQ ID NO: 14), GCGGELYKSILY (SEQ ID NO: 15) and GSITTIDVPWNVGC (SEQ ID NO: 16).

62. The kit of any one of clauses 51 to 61 wherein the peptide component of the peptidoglycan comprises an amino acid sequence of RRANAALKAGELYKSILYGC (SEQ ID NO: 1).

63. The kit of any one of clauses 51 to 62 wherein the collagen-binding synthetic peptidoglycan is DS-SILY₁₈.

64. A compound the formula

PₙGₓ wherein n is 10 to 25;

x is 1 to 10

P is a synthetic peptide of about 5 to about 40 amino acids comprising a sequence of a collagen-binding domain; and

G is a glycan.

65. The compound of clause 64 wherein n is 15 to 25.

66. The compound of clause 64 wherein n is 15 to 20.

67. The compound of clause 64 wherein n is about 18.

68. The compound of clause 64 of the formula P₁₈G₁₋₁₀.

69. The compound of clause 64 of the formula P₁₈G₁.

70. The compound of clause 64 wherein the compound is DS-SILY₁₈.

71. The compound method or kit of any of the preceding numbered clauses wherein the synthetic peptidoglycan inhibits blood cell binding to the denuded vessel.

72. The method, compound, or kit of any of the preceding clauses where the peptide component of the peptidoglycan comprises or is an amino acid sequence selected from the group consisting of RRANAALKAGELYKSILY (SEQ ID NO: 17), RLDGNEIKR (SEQ ID NO: 18), AHEEISTTNEGVM (SEQ ID NO: 19), NGVFKYRPRYFLYKHAYFYPPLKRFPVQ (SEQ ID NO: 20), CQDSETRTFY (SEQ ID NO: 5), TKKTLRT (SEQ ID NO: 21), GLRSKSKKFRRPDIQYPDATDEDITSHM (SEQ ID NO: 22), SQNPVQP (SEQ ID NO: 23), SYIRIADTNIT (SEQ ID NO: 24), SYIRIADTNIT (SEQ ID NO: 24), KELNLVYT (SEQ ID NO: 11), KELNLVYT (SEQ ID NO: 11), GELYKSILY (SEQ ID NO: 25), GSITTIDVPWNV (SEQ ID NO: 14), GCGGELYKSILY (SEQ ID NO: 15) and GSITTIDVPWNV (SEQ ID NO: 14).

### BRIEF DESCRIPTION OF THE DRAWINGS

FIG. 1 shows a schematic representation of the interaction between neighboring proteoglycans on adjacent tropocollagen strands which is important in determining the mechanical and alignment properties of collagen matrices.

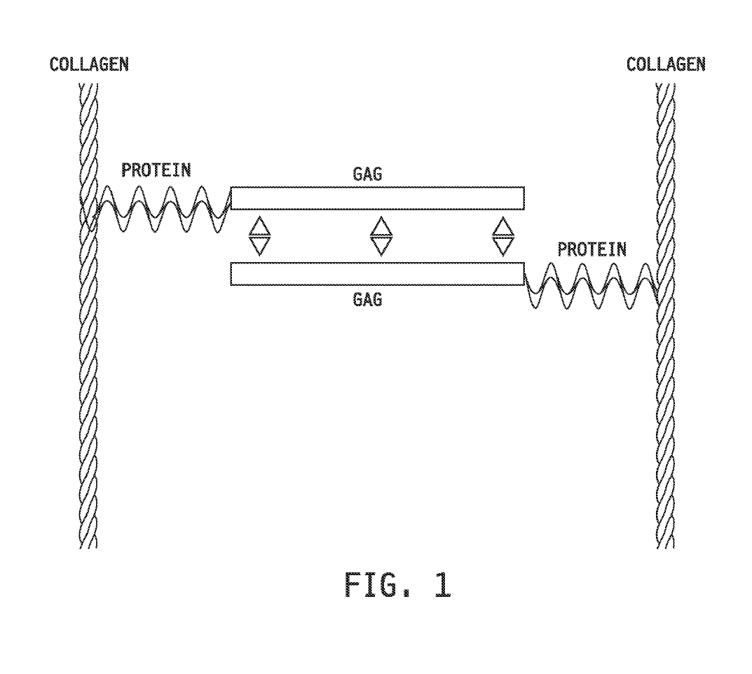

FIG. 2. AFM images made in contact mode, with a scan rate of 2 Hz with Silicon Nitride contact mode tip k=0.05 N/m tips and deflection setpoint: 0-1 Volts, of gel samples prepared as in EXAMPLE 15 (10:1 collagen:treatment) after dehydration with ethanol. Samples are for collagen alone (Collagen), and for collagen with dermatan sulfate (DS), with decorin (Decorin), dermatan sulfate-RRANAALKAGELYKSILYGC (“RRANAALKAGELYKSILYGC” disclosed as SEQ ID NO: 1) conjugate (DS-SILY) and dermatan sulfate-SYIRIADTNIT (“SYIRIADTNIT” disclosed as SEQ ID NO: 10) conjugate (DS-SYIR).

FIG. 3. Surface Plasmon Resonance scan in association mode and dissociation mode of peptide RRANAALKAGELYKSILYGC (SILY) (SEQ ID NO: 1) binding to collagen bound to CM-3 plates. SILY was dissolved in 1×HBS-EP buffer at varying concentrations from 100 μM to 1.5 μm in 2-fold dilutions.

FIG. 4. Binding of dansyl-modified peptide SILY to collagen measured in 96-well high-binding plate (black with a clear bottom (Costar)). PBS, buffer only; BSA, BSA-treated well; Collagen, collagen-treated well. Fluorescence readings were taken on an M5 Spectramax Spectrophotometer (Molecular Devices) at excitation/emission wavelengths of 335 nm/490 nm, respectively.

FIG. 5. Collagen-dansyl-modified peptide SILY binding curve derived from fluorescence data described in FIG. 4.

FIG. 6. A schematic description of the reagent, PDPH, and the chemistry of the two-step conjugation of a cysteine-containing peptide with an oxidized glycosylaminoglycoside showing the release of 2-pyridylthiol in the final step.

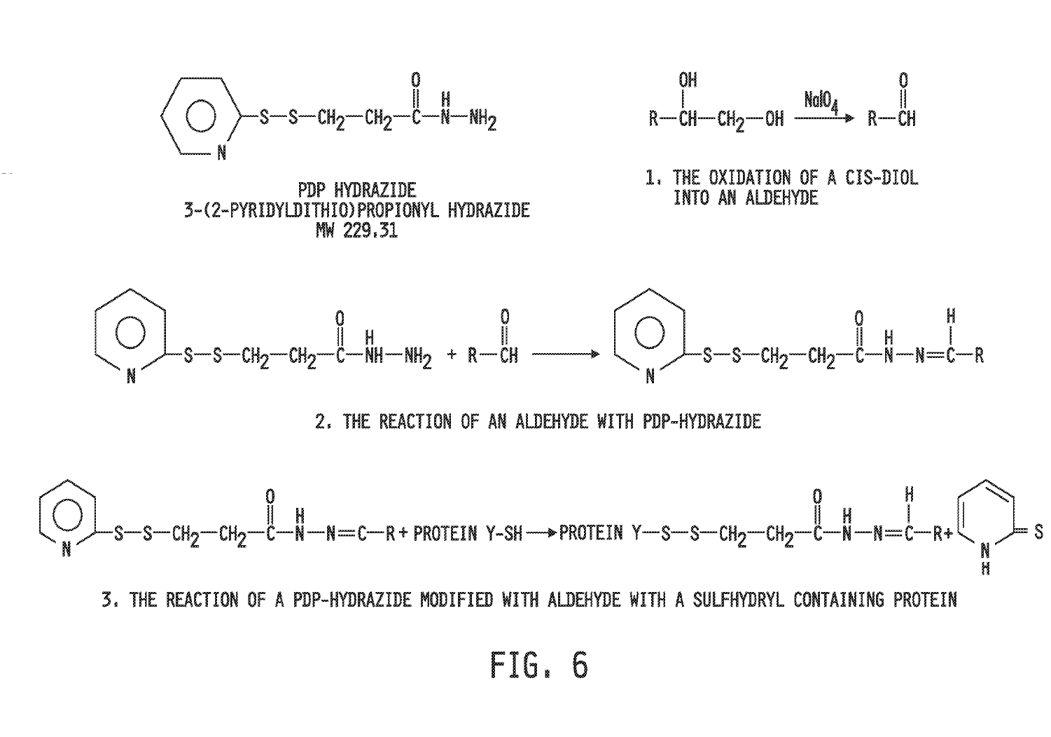

FIG. 7. Binding of dansyl-modified peptide SILY conjugated to dermatan sulfate as described herein to collagen measured in 96-well high-binding plate (black with a clear bottom (Costar)). PBS, buffer only; BSA, BSA-treated well; Collagen, collagen-treated well. Fluorescence readings were taken on an M5 Spectramax Spectrophotometer (Molecular Devices) at excitation/emission wavelengths of 335 nm/490 nm respectively.

FIG. 8. Measurement of Shear modulus of gel samples (1.5 mg/mL collagen III, 5:1 collagen:treatment) on a AR-G2 rheometer with 20 mm stainless steel parallel plate geometry (TA Instruments, New Castle, Del.), and the 20 mm stainless steel parallel plate geometry was lowered to a gap distance of 500 μm using a normal force control of 0.25N. ♦—no treatment, i.e. collagen III alone; ▪—collagen+dermatan sulfate (1:1); +—collagen+dermatan sulfate (5:1); x—collagen+dermatan sulfate-KELNLVYTGC (DS-KELN) (“KELNLVYTGC” disclosed as SEQ ID NO: 12) conjugate (1:1); ▴—collagen+dermatan sulfate-KELN conjugate (5:1); —collagen+KELNLVYTGC (KELN) (“KELNLVYTGC” disclosed as SEQ ID NO: 12) peptide.

FIG. 9. Measurement of Shear modulus of gel samples (1.5 mg/mL collagen III, 5:1 collagen:treatment) on a AR-G2 rheometer with 20 mm stainless steel parallel plate geometry (TA Instruments, New Castle, Del.), and the 20 mm stainless steel parallel plate geometry was lowered to a gap distance of 500 μm using a normal force control of 0.25N. ♦—no treatment, i.e. collagen III alone; ▪—collagen+dermatan sulfate (1:1); +—collagen+dermatan sulfate (5:1); x—collagen+dermatan sulfate-GSIT conjugate (DS-GSIT) (1:1); ▴—collagen+dermatan sulfate-GSIT conjugate (5:1); —collagen+GSITTIDVPWNVGC (GSIT) (“GSITTIDVPWNVGC” disclosed as SEQ ID NO: 16) peptide.

FIG. 10. Turbidity measurement. Gel solutions were prepared as described in EXAMPLE 15 (collagen 4 mg/mL and 10:1 collagen to treatment, unless otherwise indicated) and 50 μL/well were added at 4° C. to a 384-well plate. The plate was kept at 4° C. for 4 hours before initiating fibril formation. A SpectraMax M5 at 37° C. was used to measure absorbance at 313 nm at 30 s intervals for 6 hours. Col, no treatment, i.e., collagen alone; DS, collagen+dermatan sulfate; decorin, collagen+decorin; DS-SILY, collagen+dermatan sulfate-SILY conjugate.

FIG. 11. Cryo-Scanning Electron Microscopy images of gel structure at a magnification of 5000. Gels for cryo-SEM were formed, as described in EXAMPLE 18 (1 mg/mL collagen (Type III), 1:1 collagen:treatment), directly on the SEM stage. Regions with similar orientation were imaged for comparison across treatments. Panel a, Collagen, no treatment, i.e., collagen alone; Panel b, collagen+dermatan sulfate; Panel c, collagen+dermatan sulfate-KELN conjugate; Panel d, collagen+dermatan sulfate-GSIT conjugate.

FIG. 12. The average void space fraction measured from the Cryo-SEM images shown in FIG. 11. a) Collagen, no treatment, i.e., collagen alone; b) collagen+dermatan sulfate; c) collagen+dermatan sulfate-KELN conjugate; d) collagen+dermatan sulfate-GSIT conjugate. All differences are significant with p=0.05.

FIG. 13. Measurement of absorbance at 343 nm before treatment of oxidized heparin conjugated to PDPH, and after treatment with SILY, which releases 2-pyridylthiol from the conjugate and allows determination of the ratio of SILY peptide conjugated to oxidized heparin. The measured AA, corresponds to 5.44 SILY molecules/oxidized heparin.

FIG. 14. DS-SILY Conjugation Characterization. After 2 hours, a final ΔA_{343 nm} corresponded to 1.06 SILY molecules added to each DS molecule. Note, t=0 is an approximate zero time point due to the slight delay between addition of SILY to the DS-PDPH and measurement of the solution at 343 nm.

FIG. 15. Conjugation of Dc13 to DS. Production of pyridine-2-thione measured by an increase in absorbance at 343 nm indicates 0.99 Dc13 peptides per DS polymer chain.

FIG. 16. Microplate Fluorescence Binding of DS-ZDc13 to Collagen. DS-ZDc13 bound specifically to the collagen surface in a dose-dependent manner.

FIG. 17. Collagen Fibrillogenesis by Turbidity Measurements. DS-Dc13 delays fibrillogenesis and decreases overall absorbance in a dose-dependent manner. Free Dc13 peptide, in contrast, appears to have little effect on fibrillogenesis compared to collagen alone at the high 1:1 collagen:additive molar ratio.

FIG. 18. Average Fibril Diameter from Cryo-SEM. A. Decorin and synthetic peptidoglycans significantly decrease fibril diameter over collagen or collagen+DS. B. Compared to collagen alone, free peptide Dc13 does not affect fibril diameter while SILY results in a decrease in fibril diameter.

FIG. 19. Gel Compaction. A. and B. Days 3 and 5 respectively: Decorin and peptidoglycans are significant relative to collagen and DS, * indicates DS-Dc13 and DS are not significant at day 3. Bars indicate no significance. C. Day 7: +Decorin is significant against all samples, # DS is significant compared to collagen. D. Day 10: ++collagen and DS are significant, DS-Dc13 is significant compared to decorin and collagen.

FIG. 20. Elastin Estimate by Fastin Assay. A. DS-SILY significantly increased elastin production over all samples. DS and DS-Dc13 significantly decreased elastin production over collagen. Control samples of collagen gels with no cells showed no elastin production. B. Free peptides resulted in a slight decrease in elastin production compared to collagen, but no points were significant.

FIG. 21. SEM Images of Platelet-Rich Plasma Incubated Slides. Arrows in Heparin-SILY treatment indicate fibril-like structures unique to this treatment. Scale bar=100 μm.

FIG. 22. Fibril Density from Cryo-SEM. Fibril density, defined as the ratio of fibril containing area to void space. DS-SILY and free SILY peptide had significantly greater fibril density, while collagen had significantly lower fibril density. DS-Dc13 was not significant compared to collagen.

FIG. 23. Storage Modulus (G′) of Collagen Gels. Rheological mechanical testing of collagen gels formed with each additive at A. 5:1 B. 10:1 and C. 30:1 molar ratio of collagen:additive. Frequency sweeps from 0.1 Hz to 1.0 Hz with a controlled stress of 1.0 Pa were performed. G′avg±S.E. are presented.

FIG. 24. Cell Proliferation and Cytotoxicity Assays. No significant differences were found between all additives in A. CyQuant B. Live and C. Dead assays.

FIG. 25. Cryo-SEM Images for Fibril Density. Collagen gels formed in the presence of each additive at a 10:1 molar ratio of collagen:additive. DS, Decorin, or peptidoglycans. Free Peptides. Images are taken at 10,000×, Scale bar=5 p.m.

FIG. 26. AFM Images of Collagen Gels. Collagen gels were formed in the presence of each additive at a 10:1 molar ratio of collagen:additive. D-banding is observed for all additives. Images are 1 μm².

FIG. 27. Inhibition of Platelet Activation. Measured by determining the release of activation factors Platelet Factor 4 (PF-4) and β-thromboglobulin (Nap-2). Collagen immobilized on the surface of a 96-well plate was pre-incubated with each treatment and subsequently incubated with platelet rich plasma (PRP). Values are reported as a percentage of activation factor released by the treatment compared to the amount of activation factor released by the control treatment (phosphate buffered saline, PBS). The * indicates that the difference is significant vs. collagen surface with no treatment (phosphate buffered saline, PBS). Dex, dextran; Dex-SILY9, dextran-(SILY)₉ conjugate; Hep, heparin; Hep-SILY, heparin-SILY conjugate; HA, hyaluronan; HA-SILY, hyaluronan-SILY conjugate; SILY, SILY peptide. Due to solubility limits, Hep, Hep-SILY, HA, and HA-SILY were incubated at 25 μM. All other treatments were at 50 μM (after the treatment was removed, the plates were washed with PBS<1 min, before addition of PRP). Hep and HA (hyaluronic acid) conjugates contained approximately 4 peptides per polysaccharide.

FIG. 28. Inhibition of Platelet Activation. Measured by determining the release of activation factors Platelet Factor 4 (PF-4) and β-thromboglobulin (Nap-2). Collagen immobilized on the surface of a 96-well plate was pre-incubated with each treatment and subsequently incubated with platelet rich plasma (PRP). Values are reported as a percentage of activation factor released by the treatment compared to the amount of activation factor released by the control treatment (phosphate buffered saline, PBS). Dex, dextran; Dex-SILY6, dextran-(SILY)₆ conjugate; Hep, heparin; Hep-GSIT, heparin-GSIT conjugate; GSIT, GSIT peptide; SILY, SILY peptide. The values measured for all treatments are significant vs. PBS. Dex, SILY, and Dex-SILY6 are at 25 μM, all other treatments are at 50 μM. The ** indicates that the value for the Hep-GSIT treatment was significant vs. the values for the Hep treatment, similarly the value for the Dex-SILY6 treatment was significant vs. the value for the Dex treatment for PF4. (After the treatment was removed the plates were rinsed for 20 min). Hep conjugates contained approximately 4 peptides per polysaccharide.

FIG. 29. Inhibition of Platelet Binding to Collagen by Colorimetric Assay. Collagen immobilized on the surface of a 96-well plate was pre-incubated with each treatment and subsequently incubated with platelet rich plasma (PRP). Microplate assay prepared as described was pre-incubated with treatments Collagen, PBS only; Dextran; Dex-SILY6, dextran-(SILY)₆; SILY, SILY peptide. * Significant vs. collagen (no treatment).

FIG. 30. Fluorescence image of adhered platelets. Adhered platelets were fixed with 4% paraformaldehyde, permeabilized with 0.1% Triton X-100, and platelet actin was labeled with phalloidin-AlexaFluor 488. The adhered platelets were imaged using an upright fluorescent microscope using a DAPI filter. No treatment, i.e. collagen treated with PBS.

FIG. 31. Fluorescence image of adhered platelets. Adhered platelets were fixed with 4% paraformaldehyde, permeabilized with 0.1% Triton X-100, and platelet actin was labeled with phalloidin-AlexaFluor 488. The adhered platelets were imaged using an upright fluorescence microscope using a DAPI filter. Treatment: dextran.

FIG. 32. Fluorescence image of adhered platelets. Adhered platelets were fixed with 4% paraformaldehyde, permeabilized with 0.1% Triton X-100, and platelet actin was labeled with phalloidin-AlexaFluor 488. The adhered platelets were imaged using an upright fluorescence microscope using a DAPI filter. Treatment: dextran-SILY9 conjugate.

FIG. 33. Fluorescence image of adhered platelets. Adhered platelets were fixed with 4% paraformaldehyde, permeabilized with 0.1% Triton X-100, and platelet actin was labeled with phalloidin-AlexaFluor 488. The adhered platelets were imaged using an upright fluorescence microscope using a DAPI filter. Treatment: hyaluronan.

FIG. 34. Fluorescence image of adhered platelets. Adhered platelets were fixed with 4% paraformaldehyde, permeabilized with 0.1% Triton X-100, and platelet actin was labeled with phalloidin-AlexaFluor 488. The adhered platelets were imaged using an upright fluorescence microscope using a DAPI filter. Treatment: hyaluronan-SILY conjugate.

FIG. 35. Fluorescence image of adhered platelets. Adhered platelets were fixed with 4% paraformaldehyde, permeabilized with 0.1% Triton X-100, and platelet actin was labeled with phalloidin-AlexaFluor 488. The adhered platelets were imaged using an upright fluorescence microscope using a DAPI filter. Treatment: heparin.

FIG. 36. Fluorescence image of adhered platelets. Adhered platelets were fixed with 4% paraformaldehyde, permeabilized with 0.1% Triton X-100, and platelet actin was labeled with phalloidin-AlexaFluor 488. The adhered platelets were imaged using an upright fluorescence microscope using a DAPI filter. Treatment: heparin-SILY conjugate.

FIG. 37. Fluorescence image of adhered platelets. Adhered platelets were fixed with 4% paraformaldehyde, permeabilized with 0.1% Triton X-100, and platelet actin was labeled with phalloidin-AlexaFluor 488. The adhered platelets were imaged using an upright fluorescence microscope using a DAPI filter. Treatment: SILY peptide.

FIG. 38. Collagen Degradation Determined by Hydroxyproline. Treatments: Ctrl, no cells added; Col, collagen without added treatment; DS, dermatan sulfate; Decorin; DS-SILY, dermatan sulfate-SILY conjugate; DS-Dc13, dermatan sulfate-Dc13 conjugate; SILY, SILY peptide; Dc13, Dc13 peptide.

FIG. 39. Inhibition of Platelet Activation. Measured by determining the release of activation factors Platelet Factor 4 (PF-4) and β-thromboglobulin (Nap-2). Type I and III collagen gels on the surface of a 96-well plate were pre-incubated with each treatment and subsequently incubated with PRP. Platelet activation was measured by the release of activation factors PF-4 and Nap-2. Treatments: PBS, buffer alone; Dex, dextran; Dex-SILY, dextran-SILY conjugate; Dex-GSIT, dextran-GSIT conjugate; Dex-KELN, dextran-KELN conjugate; Dex-Dc13, dextran-Dc13 conjugate; SILY, SILY peptide; GSIT, GSIT peptide; KELN, KELN peptide; Dc13, Dc13 peptide; Dex-SILY+Dex-GSIT; combination of dextran-SILY conjugate and dextran-GSIT conjugate; SILY+GSIT; combination of SILY peptide and GSIT peptide. * Indicates the results are significant vs. collagen surface with no treatment (PBS). ** Indicates the results are also significant vs. collagen surface with Dex. *** Indicates the results are also significant vs. collagen surface with corresponding peptide control. All peptidoglycans caused significant decrease in NAP-2 release compared to no treatment (PBS) or dextran treatment, while Dex-GSIT additionally decreased release over its peptide control (GSIT). Dex-GSIT and Dex-KELN significantly decreased PF-4 release relative to no treatment (PBS) and dextran treatment, while Dex-Dc13 significantly decreased PF-4 release over no treatment (PBS).

FIG. 40. Inhibition of Platelet Binding to Collagen (Adhesion) by Colorimetric Assay. Treatments: PBS, buffer alone; Dex, dextran; Dex-SILY, dextran-SILY conjugate; Dex-GSIT, dextran-GSIT conjugate; Dex-KELN, dextran-KELN conjugate; Dex-Dc13, dextran-Dc13 conjugate; SILY, SILY peptide; GSIT, GSIT peptide; KELN, KELN peptide; Dc13, Dc13 peptide; Dex-SILY+Dex-GSIT; combination of dextran-SILY conjugate and dextran-GSIT conjugate; SILY+GSIT; combination of SILY peptide and GSIT peptide. * Significant vs. Collagen surface with no treatment (PBS). ** Also significant vs. collagen surface with Dex. *** Also significant vs. collagen surface with corresponding peptide control. Dex-SILY and Dex-KELN had significantly decreased platelet adherence as compared to no treatment (PBS) or Dextran treatment, while Dex-GSIT additionally decreased platelet adherence over its peptide control treatment (GSIT).

FIG. 41 shows the purification of DS-BMPH. The number of BMPH crosslinkers attached to DS is determined by calculating the excess BMPH which is then subtracted from the known amount added, which yields the amount reacted with oxidized DS. Excellent separation of the two molecular species is achieved under the purification procedures, which is shown by the wide separation of peaks.

FIG. 42 shows periodate oxidation. By increasing the amount of sodium meta-periodate during oxidation of DS, the number of BMPH crosslinkers per DS chain increases linearly.

FIG. 43 shows peptidoglycan binding affinity. Biotin labeled peptidoglycans DS-SILY₄ and DS-SILY₁₈ were synthesized and incubated on a fibrillar collagen surface. After washing, the bound peptidoglycan was detected and saturation binding curves were fitted to calculate the binding affinities. DS-SILY₄ and DS-SILY₁₈ binding to collagen with K_{D}=118 nM and 24 nM respectively, demonstrating that increasing the number of attached peptides increases the affinity of the peptidoglycan to collagen. In addition a greater number of peptides increases the amount of peptidoglycan that binds to the surface, which is noted by the increase in absorbance. Note DS-SILY₁₈ does not contain more biotin labeled peptides than DS-SILY₄.

FIG. 44 shows the percent decrease in release of activation factors PF4 and NAP2 as compared to untreated collagen surfaces (NT). DS, SILY, or DS-SILY was incubated for 15 min at a concentration of 50 μM and then rinsed from the collagen surface for 24 hours. * indicates significance to NT, ** indicates significance to NT, and DS, and SILY α=0.05.

FIG. 45 shows the inhibition of platelet activation. Fibrillar collagen surfaces were incubated with varying concentrations of peptidoglycan DS-SILY₁₈. Unbound peptidoglycan was rinsed from the surface over 24 hours. Human platelets were then incubated on the surface and activation was measured by release of PF-4 and Nap-2 following FDA guidelines. Maximal inhibition of platelet activation was achieved at 10 μM concentrations.

FIG. 46 shows the diffusion of DS-SILY₁₈ from a fibrillar collagen surface. Labeled DS-SILY₁₈ was bound on a collagen surface as described and incubated at 37° C. with extensive rinsing for up to 11 days. Detection of the peptidoglycan at various time points showed that it diffuses from the surface over time but even after 1 week, the equivalent of approximately 10 nM remained bound.

FIG. 47 shows endothelial cell proliferation. Proliferation was measured in the presence of varying concentrations of peptidoglycan to determine whether the peptidoglycan had an adverse effect on endothelial regrowth. At the highest concentration tested, a significant increase in cell proliferation was observed. * indicates significance α=0.05, n=6, presented as average+std. dev.

FIG. 48 shows endothelial migration on treated collagen surfaces. To more closely mimic the environment of a denuded vessel with exposed collagen, endothelial growth onto collagen with varying concentrations of bound peptidoglycan was tested. At higher peptidoglycan concentrations there was a significant increase in endothelial cell migration. * indicates significance α=0.05, n=6, presented as avg.+std dev.

FIG. 49 shows platelet binding to collagen under flow. Human platelet-rich plasma was tested under flow for platelet binding on fibrillar collagen surfaces. Treatment conditions DS-SILY treated (Panel A) or untreated (Panel B) collagen surfaces show significantly fewer bound platelets on the peptidoglycan treated surface.

FIG. 50 shows a schematic representation of peptidoglycan inhibition of platelet binding and activation on collagen of denuded endothelium.

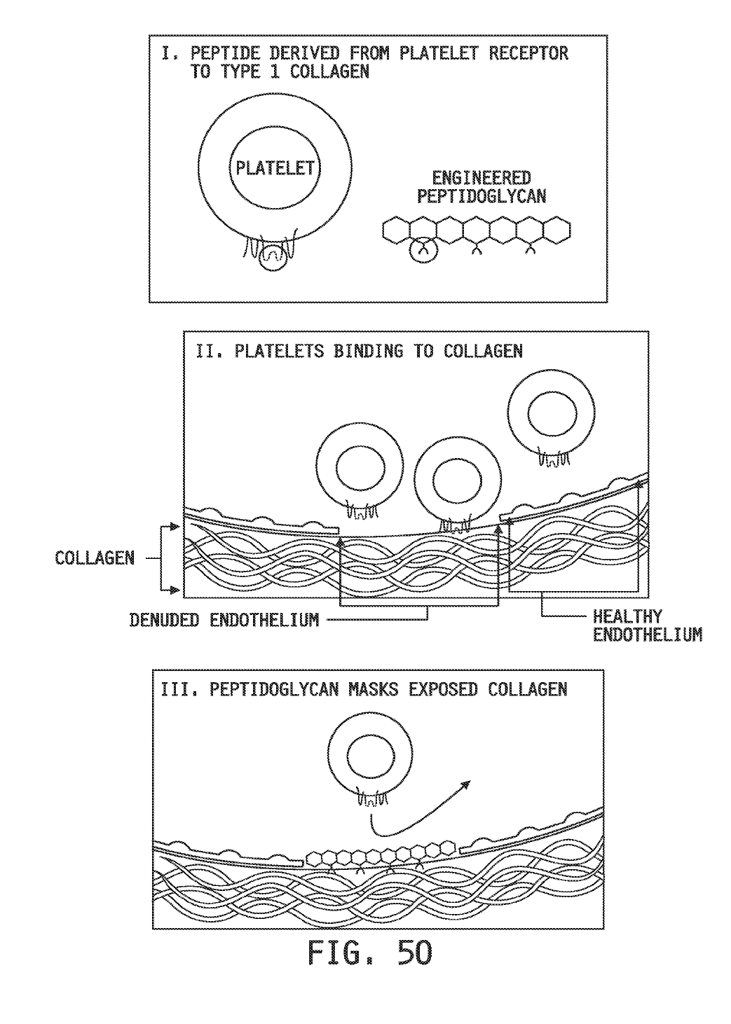

FIG. 51 shows the quantification of inhibited platelet binding by vasospasm. Panel A shows a representative angiography profile of treated and untreated balloon injured vessels. Vasospasm is apparent in the untreated vessel while the peptidoglycan treatment does not exhibit vasospasm. Panel B shows vasospasm quantified by measuring the % vessel occlusion using angiography data. A total of 12 balloon injuries, 7 untreated and 5 treated, were analyzed. * indicates significance compared to untreated p=0.005.

FIG. 52 shows histological evaluation of balloon injured vessels using Verhoff-Van Gieson staining. Intimal hyperplasia is apparent in the sham control (panel A) as noted by growth from the internal elastic lamina. In peptidoglucan treated vessels, intimal hyperplasia is absent (panel B).

FIG. 53 shows denuded arteries incubated with 1×PBS (Control) or labeled peptidoglycan (Peptidoglycan (10 μM DS-SILY_{18-biotin}).

FIG. 54 A and B show inhibition by DS-SILY₁₈ of whole blood binding to collagen under flow.

### DETAILED DESCRIPTION OF THE ILLUSTRATIVE EMBODIMENTS

As used in accordance with this invention, a “collagen-binding synthetic peptidoglycan” means a collagen-binding conjugate of a glycan with a synthetic peptide. The “collagen-binding synthetic peptidoglycans” can have amino acid homology with a portion of a protein or a proteoglycan not normally involved in collagen fibrillogenes or can have amino acid homology to a portion of a protein or to a proteoglycan normally involved in collagen fibrillogenesis.

In an illustrative embodiment, these collagen-binding synthetic proteoglycans can be used in vascular intervention procedures including, for example, to prevent any one or a combination of platelet binding to exposed collagen of the denuded endothelium, platelet activation, thrombosis, inflammation resulting from denuding the endothelium, intimal hyperplasia, and vasospasm. The collagen-binding synthetic peptidoglycans described herein can also stimulate endothelial cell proliferation and can bind to collagen in a denuded vessel.

In various embodiments described herein, the collagen-binding synthetic peptidoglycans described comprise synthetic peptides of about 5 to about 40 amino acids. In some embodiments, these peptides have homology to the amino acid sequence of a small leucine-rich proteoglycan, a platelet receptor sequence, or a protein that regulates collagen fibrillogenesis. In various embodiments the synthetic peptide comprises an amino acid sequence selected from the group consisting of RRANAALKAGELYKSILYGC (SEQ ID NO: 1), RLDGNEIKRGC (SEQ ID NO: 2), AHEEISTTNEGVMGC (SEQ ID NO: 3), GCGGELYKSILY (SEQ ID NO: 15), NGVFKYRPRYFLYKHAYFYPPLKRFPVQGC (SEQ ID NO: 4), CQDSETRTFY (SEQ ID NO: 5), TKKTLRTGC (SEQ ID NO: 6), GLRSKSKKFRRPDIQYPDATDEDITSHMGC (SEQ ID NO: 7), SQNPVQPGC (SEQ ID NO: 8), SYIRIADTNITGC (SEQ ID NO: 9), SYIRIADTNIT (SEQ ID NO: 10), KELNLVYT (SEQ ID NO: 11), KELNLVYTGC (SEQ ID NO: 12), GSITTIDVPWNV (SEQ ID NO: 14), GELYKSILYGC (SEQ ID NO: 13), and GSITTIDVPWNVGC (SEQ ID NO: 16). In another embodiment, the synthetic peptide can comprise or can be an amino acid sequence selected from the group consisting of RRANAALKAGELYKSILYGC (SEQ ID NO: 1), RLDGNEIKRGC (SEQ ID NO: 2), AHEEISTTNEGVMGC (SEQ ID NO: 3), NGVFKYRPRYFLYKHAYFYPPLKRFPVQGC (SEQ ID NO: 4), CQDSETRTFY (SEQ ID NO: 5), TKKTLRTGC (SEQ ID NO: 6), GLRSKSKKFRRPDIQYPDATDEDITSHMGC (SEQ ID NO: 7), SQNPVQPGC (SEQ ID NO: 8), SYIRIADTNITGC (SEQ ID NO: 9), SYIRIADTNIT (SEQ ID NO: 10), KELNLVYT (SEQ ID NO: 11), KELNLVYTGC (SEQ ID NO: 12), GSITTIDVPWNV (SEQ ID NO: 14), GELYKSILYGC (SEQ ID NO: 13), GSITTIDVPWNVGC (SEQ ID NO: 16), GCGGELYKSILY (SEQ ID NO: 15), and an amino acid sequence with 80%, 85%, 90%, 95%, or 98% homology with to any of these sixteen amino acid sequences. In another embodiment, the synthetic peptide can comprise or can be an amino acid sequence selected from the group consisting of RRANAALKAGELYKSILY (SEQ ID NO: 17), RLDGNEIKR (SEQ ID NO: 18), AHEEISTTNEGVM (SEQ ID NO: 19), NGVFKYRPRYFLYKHAYFYPPLKRFPVQ (SEQ ID NO: 20), CQDSETRTFYGC (SEQ ID NO: 26), TKKTLRT (SEQ ID NO: 21), GLRSKSKKFRRPDIQYPDATDEDITSHM (SEQ ID NO: 22), SQNPVQP (SEQ ID NO: 23), SYIRIADTNIT (SEQ ID NO: 24), SYIRIADTNITGC (SEQ ID NO: 9), KELNLVYTGC (SEQ ID NO: 12), KELNLVYT (SEQ ID NO: 11), GSITTIDVPWNVGC (SEQ ID NO: 16), GELYKSILY (SEQ ID NO: 25), GSITTIDVPWNV (SEQ ID NO: 14), GCGGELYKSILYGC (SEQ ID NO: 27), and an amino acid sequence with 80%, 85%, 90%, 95%, or 98% homology with to any of these sixteen amino acid sequences. The synthetic peptide can also be any peptide of 5 to 40 amino acids selected from peptides that have collagen-binding activity and that are 80%, 85%, 90%, 95%, 98%, or 100% homologous with the collagen-binding domain(s) of the von Willebrand factor or a platelet collagen receptor as described in Chiang, et al., J. Biol. Chem. 277: 34896-34901 (2002), Huizinga, et al., Structure 5: 1147-1156 (1997), Romijn, et al., J. Biol. Chem. 278: 15035-15039 (2003), and Chiang, et al., Cardio. & Haemato. Disorders-Drug Targets 7: 71-75 (2007), each incorporated herein by reference.

The glycan (e.g. glycosaminoglycan, abbreviated GAG, or polysaccharide) attached to the synthetic peptide can be selected from the group consisting alginate, agarose, dextran, chondroitin, dermatan, dermatan sulfate, heparan, heparin, keratin, and hyaluronan. In one embodiment, the glycan is selected from the group consisting of dermatan sulfate, dextran, and heparin. In another illustrative embodiment the glycan is dermatan sulfate. The collagen-binding synthetic proteoglycan in any of these embodiments can be used to inhibit platelet binding to exposed collagen of the denuded endothelium, platelet activation, thrombosis, inflammation resulting from denuding the endothelium, intimal hyperplasia, and vasospasm during a vascular intervention procedure. The collagen-binding synthetic peptidoglycans described herein can also stimulate endothelial cell proliferation and can bind to collagen in a denuded vessel.

In one illustrative aspect, the collagen-binding synthetic peptidoglycan may be sterilized. As used herein “sterilization” or “sterilize” or “sterilized” means disinfecting the collagen-binding synthetic peptidoglycans by removing unwanted contaminants including, but not limited to, endotoxins and infectious agents.

In various illustrative embodiments, the collagen-binding synthetic peptidoglycan can be disinfected and/or sterilized using conventional sterilization techniques including propylene oxide or ethylene oxide treatment, gas plasma sterilization, gamma radiation, electron beam, and/or sterilization with a peracid, such as peracetic acid. Sterilization techniques which do not adversely affect the structure and biotropic properties of the collagen-binding synthetic peptidoglycan can be used. Illustrative sterilization techniques are exposing the collagen-binding synthetic peptidoglycan to peracetic acid, 1-4 Mrads gamma irradiation (or 1-2.5 Mrads of gamma irradiation), ethylene oxide treatment, sterile filtration, or gas plasma sterilization. In one embodiment, the collagen-binding synthetic peptidoglycan can be subjected to one or more sterilization processes. Another illustrative embodiment is subjecting the collagen-binding synthetic proteoglycan to sterile filtration. The collagen-binding synthetic peptidoglycan may be wrapped in any type of container including a plastic wrap or a foil wrap, and may be further sterilized.

In various embodiments described herein, the collagen-binding synthetic peptidoglycans can be combined with minerals, amino acids, sugars, peptides, proteins, vitamins (such as ascorbic acid), or laminin, collagen, fibronectin, hyaluronic acid, fibrin, elastin, or aggrecan, or growth factors such as epidermal growth factor, platelet-derived growth factor, transforming growth factor beta, or fibroblast growth factor, and glucocorticoids such as dexamethasone or viscoelastic altering agents, such as ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic.

In various embodiments described herein, a kit is provided comprising one or more collagen-binding synthetic peptidoglycans. The kit itself can be within a container of any type, and the kit can contain instructions for use of the components of the kit. In one embodiment, the kit comprises a vessel, vial, container, bag, or wrap, for example, containing a collagen-binding synthetic peptidoglycan. In another embodiment, the kit comprises a vessel or separate vessels (e.g., a vial, container, bag, or wrap), each containing one of the following components: a buffer and one or more types of collagen-binding synthetic peptidoglycans. In any of these embodiments, the kits can further comprise a buffer, a sterilizing or disinfecting agent, non-collagenous proteins or polysaccharides, and/or instructional materials describing methods for using the kit reagents. In any of these embodiments, the kit can contain a component selected from the group consisting of a catheter, a stent, a balloon, and a combination thereof. The collagen-binding synthetic peptidoglycan can be lyophilized, for example, in a buffer or in water.

In any of the embodiments herein described, the collagen-binding synthetic peptidoglycan can be a compound of any of the following formulas

A) PₙGₓ wherein n is 1 to 50;
wherein x is 1 to 10;
wherein P is a synthetic peptide of about 5 to about 40 amino acids comprising a sequence of a collagen-binding domain; and
wherein G is a glycan.
OR

B) (PₙL)ₓG wherein n is 1 to 7;
wherein x is 1 to 10;
wherein P is a synthetic peptide of about 5 to about 40 amino acids comprising a sequence of a collagen-binding domain;
wherein L is a linker; and
wherein G is a glycan.
OR

C) P(LGₙ)ₓ wherein n is 1 to 5;
wherein x is 1 to 10;
wherein P is a synthetic peptide of about 5 to about 40 amino acids comprising a sequence of a collagen-binding domain;
wherein L is a linker; and
wherein G is a glycan.

In any of the above described formulas, n can be 1 to 5, 1 to 10, 1 to 15, 1 to 20, 1 to 25, 1 to 30, 1 to 35, 1 to 40, 1 to 45, 1 to 50, 10 to 25, 15 to 25, 15 to 20, 18, or about 18.

In alternative embodiments, the collagen-binding synthetic peptidoglycan can be a compound of any of the following formulas

A) PₙGₓ wherein n is MWG/1000;
wherein MWG is the molecular weight of G rounded to the nearest 1 kDa;
wherein x is 1 to 10;
wherein P is a synthetic peptide of about 5 to about 40 amino acids comprising a sequence of a collagen-binding domain; and
wherein G is a glycan.
OR

B) (PₙL)ₓG wherein n is 1 to 7;
wherein x is MWG/1000;
wherein MWG is the molecular weight of G rounded to the nearest 1 kDa;
wherein P is a synthetic peptide of about 5 to about 40 amino acids comprising a sequence of a collagen-binding domain;
wherein L is a linker; and
wherein G is a glycan.

In various embodiments described herein, a collagen-binding synthetic peptidoglycan comprising a synthetic peptide of about 5 to about 40 amino acids with amino acid sequence homology to a collagen binding peptide (e.g. a portion of an amino acid sequence of a collagen binding protein or proteoglycan) conjugated to alginate, agarose, dextran, chondroitin, dermatan, dermatan sulfate, heparan, heparin, keratin, and hyaluronan. In one embodiment, the glycan is selected from the group consisting of dermatan sulfate, dextran, hyaluronan, and heparin. In another illustrative embodiment the glycan is dermatan sulfate. In yet another embodiment, the glycan is dermatan sulfate with 18 peptides of the sequence RRANAALKAGELYKSILYGC (SEQ ID NO: 1) linked to the glycan (i.e., DS-SILY₁₈). In yet another embodiment, the glycan is dermatan sulfate with 18 peptides comprising the sequence RRANAALKAGELYKSILY (SEQ ID NO: 17) linked to the glycan. The collagen-binding synthetic proteoglycan in any of these embodiments can be used to inhibit platelet binding to exposed collagen of the denuded endothelium, inhibit binding of other cells in blood to exposed collagen of the denuded epithelium, inhibit platelet activation, inhibit thrombosis, inhibit inflammation resulting from denuding the endothelium, inhibit intimal hyperplasia, and/or inhibit vasospasm. The collagen-binding synthetic peptidoglycans described herein can also stimulate endothelial cell proliferation and can bind to collagen in a denuded vessel. In any of these embodiments, these aforementioned effects can occur during a vascular intervention procedure, such as a catheter-based procedure. In any of these embodiments, any of the above-described compounds can be used.

In another illustrative embodiment, any of the compounds described above as embodiments A, B, or C or alternative embodiments A or B can inhibit platelet binding to exposed collagen of the denuded endothelium, platelet activation, thrombosis, inflammation resulting from denuding the endothelium, intimal hyperplasia, and/or vasospasm, or can stimulate endothelial cell proliferation or can bind to collagen in a denuded vessel. In another illustrative embodiment, during a vascular intervention procedure, any of the compounds described above as embodiments A, B, or C or alternative embodiments A or B, can inhibit platelet binding to exposed collagen of the denuded endothelium, platelet activation, thrombosis, inflammation resulting from denuding the endothelium, intimal hyperplasia, and/or vasospasm, or can stimulate endothelial cell proliferation or can bind to collagen in a denuded vessel. In another illustrative embodiment, during a vascular intervention procedure, any of the compounds described above as embodiments A, B, or C or alternative embodiments A or B, can inhibit platelet binding to exposed collagen of the denuded endothelium, platelet activation, intimal hyperplasia, and/or vasospasm, or can bind to collagen in a denuded vessel.

In another illustrative embodiment, DS-SILY₁₈ can inhibit platelet binding to exposed collagen of the denuded endothelium, platelet activation, thrombosis, inflammation resulting from denuding the endothelium, intimal hyperplasia, and/or vasospasm, or can stimulate endothelial cell proliferation or can bind to collagen in a denuded vessel. In another illustrative embodiment, during a vascular intervention procedure, DS-SILY₁₈ can inhibit platelet binding to exposed collagen of the denuded endothelium, platelet activation, thrombosis, inflammation resulting from denuding the endothelium, intimal hyperplasia, and/or vasospasm, or can stimulate endothelial cell proliferation or can bind to collagen in a denuded vessel. In another illustrative embodiment, during a vascular intervention procedure, DS-SILY₁₈ can inhibit platelet binding to exposed collagen of the denuded endothelium, platelet activation, intimal hyperplasia, and/or vasospasm, or can bind to collagen in a denuded vessel.

In various embodiments described herein, the synthetic peptides described herein can be modified by the inclusion of one or more conservative amino acid substitutions. As is well known to those skilled in the art, altering any non-critical amino acid of a peptide by conservative substitution should not significantly alter the activity of that peptide because the side-chain of the replacement amino acid should be able to form similar bonds and contacts to the side chain of the amino acid which has been replaced.

Non-conservative substitutions are possible provided that these do not excessively affect the collagen binding activity of the peptide and/or reduce its effectiveness in inhibiting platelet activation, platelet binding to exposed collagen of the denuded endothelium, platelet activation, thrombosis, inflammation resulting from denuding the endothelium, intimal hyperplasia, and/or vasospasm, or its effectiveness in stimulating endothelial cell proliferation or in binding to collagen in a denuded vessel.

As is well-known in the art, a “conservative substitution” of an amino acid or a “conservative substitution variant” of a peptide refers to an amino acid substitution which maintains: 1) the secondary structure of the peptide; 2) the charge or hydrophobicity of the amino acid; and 3) the bulkiness of the side chain or any one or more of these characteristics. Illustratively, the well-known terminologies “hydrophilic residues” relate to serine or threonine. “Hydrophobic residues” refer to leucine, isoleucine, phenylalanine, valine or alanine, or the like. “Positively charged residues” relate to lysine, arginine, ornithine, or histidine. “Negatively charged residues” refer to aspartic acid or glutamic acid. Residues having “bulky side chains” refer to phenylalanine, tryptophan or tyrosine, or the like. A list of illustrative conservative amino acid substitutions is given in TABLE 1.

TABLE 1

| For Amino Acid | Replace With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

In the various conservative amino acid substitution embodiments described herein, a collagen-binding synthetic peptidoglycan comprising a synthetic peptide of about 5 to about 40 amino acids with amino acid sequence homology to a collagen binding peptide (e.g. a portion of an amino acid sequence of a collagen binding protein or proteoglycan) conjugated to a glycan selected from the group consisting of alginate, agarose, dextran, chondroitin, dermatan, dermatan sulfate, heparan, heparin, keratin, and hyaluronan can be used. In one embodiment, the glycan is selected from the group consisting of dermatan sulfate, dextran, hyaluronan, and heparin. In another illustrative embodiment the glycan is dermatan sulfate. In yet another embodiment, the glycan is dermatan sulfate with 18 peptides of the sequence RRANAALKAGELYKSILYGC (SEQ ID NO: 1) linked to the glycan and this sequence can be conservatively substituted. The collagen-binding synthetic proteoglycan in any of these conservative substitution embodiments can be used to inhibit platelet binding to exposed collagen of the denuded endothelium, platelet activation, thrombosis, inflammation resulting from denuding the endothelium, intimal hyperplasia, and/or vasospasm. The collagen-binding synthetic peptidoglycans described herein with conservative amino acid substitutions can also stimulate endothelial cell proliferation and can bind to collagen in a denuded vessel. In any of these embodiments, these aforementioned effects can occur during a vascular intervention procedure, such as a catheter-based procedure. In any of these conservative substitution embodiments, any of the above-described compounds can be used.

In another illustrative embodiment, any of the compounds selected from the group consisting of RRANAALKAGELYKSILYGC (SEQ ID NO: 1), RLDGNEIKRGC (SEQ ID NO: 2), AHEEISTTNEGVMGC (SEQ ID NO: 3), NGVFKYRPRYFLYKHAYFYPPLKRFPVQGC (SEQ ID NO: 4), CQDSETRTFY (SEQ ID NO: 5), TKKTLRTGC (SEQ ID NO: 6), GLRSKSKKFRRPDIQYPDATDEDITSHMGC (SEQ ID NO: 7), SQNPVQPGC (SEQ ID NO: 8), SYIRIADTNITGC (SEQ ID NO: 9), SYIRIADTNIT (SEQ ID NO: 10), KELNLVYT (SEQ ID NO: 11), KELNLVYTGC (SEQ ID NO: 12), GSITTIDVPWNV (SEQ ID NO: 14), GELYKSILYGC (SEQ ID NO: 13), GSITTIDVPWNVGC (SEQ ID NO: 16), and GCGGELYKSILY (SEQ ID NO: 15) having conservative amino acid substitutions can be used. In any of these embodiments the compounds with conservative amino acid substitutions can inhibit platelet binding to exposed collagen of the denuded endothelium, platelet activation, thrombosis, inflammation resulting from denuding the endothelium, intimal hyperplasia, and/or vasospasm, or can stimulate endothelial cell proliferation or can bind to collagen in a denuded vessel. In another illustrative embodiment, during a vascular intervention procedure, any of these compounds with conservative amino acid substitutions can inhibit platelet binding to exposed collagen of the denuded endothelium, platelet activation, thrombosis, inflammation resulting from denuding the endothelium, intimal hyperplasia, and/or vasospasm, or can stimulate endothelial cell proliferation or can bind to collagen in a denuded vessel. In another illustrative embodiment, during a vascular intervention procedure, any of the compounds with conservative amino acid substitutions described in this paragraph can inhibit platelet binding to exposed collagen of the denuded endothelium, platelet activation, intimal hyperplasia, and/or vasospasm, or can bind to collagen in a denuded vessel.

In various embodiments described herein, the synthetic peptide is synthesized according to solid phase peptide synthesis protocols that are well known by persons of skill in the art. In one embodiment a peptide precursor is synthesized on a solid support according to the well-known Fmoc protocol, cleaved from the support with trifluoroacetic acid and purified by chromatography according to methods known to persons skilled in the art.

In various embodiments described herein, the synthetic peptide is synthesized utilizing the methods of biotechnology that are well known to persons skilled in the art. In one embodiment a DNA sequence that encodes the amino acid sequence information for the desired peptide is ligated by recombinant DNA techniques known to persons skilled in the art into an expression plasmid (for example, a plasmid that incorporates an affinity tag for affinity purification of the peptide), the plasmid is transfected into a host organism for expression, and the peptide is then isolated from the host organism or the growth medium according to methods known by persons skilled in the art (e.g., by affinity purification). Recombinant DNA technology methods are described in Sambrook et al., “Molecular Cloning: A Laboratory Manual”, 3rd Edition, Cold Spring Harbor Laboratory Press, (2001), incorporated herein by reference, and are well-known to the skilled artisan.

In various embodiments described herein, the synthetic peptide is conjugated to a glycan by reacting a free amino group of the peptide with an aldehyde function of the glycan in the presence of a reducing agent, utilizing methods known to persons skilled in the art, to yield the peptide glycan conjugate. In one embodiment an aldehyde function of the glycan (e.g. polysaccharide or glycosaminoglycan) is formed by reacting the glycan with sodium metaperiodate according to methods known to persons skilled in the art.

In one embodiment, the synthetic peptide is conjugated to a glycan by reacting an aldehyde function of the glycan with 3-(2-pyridyldithio)propionyl hydrazide (PDPH) to form an intermediate glycan and further reacting the intermediate glycan with a peptide containing a free thiol group to yield the peptide glycan conjugate. In yet another embodiment, the sequence of the peptide may be modified to include a glycine-cysteine segment to provide an attachment point for a glycan or a glycan-linker conjugate.

In any of the embodiments described herein, the synthetic peptide is conjugated to a glycan by reacting an aldehyde function of the glycan with a crosslinker, e.g., 3-(2-pyridyldithio)propionyl hydrazide (PDPH), to form an intermediate glycan and further reacting the intermediate glycan with a peptide containing a free thiol group to yield the peptide glycan conjugate. In any of the various embodiments described herein, the sequence of the peptide may be modified to include a glycine-cysteine segment to provide an attachment point for a glycan or a glycan-linker conjugate. In any of the embodiments described herein, the crosslinker can be N-[β-Maleimidopropionic acid]hydrazide (BMPH).

Although specific embodiments have been described in the preceding paragraphs, the collagen-binding synthetic peptidoglycans described herein can be made by using any art-recognized method for conjugation of the peptide to the glycan (e.g. polysaccharide or glycosaminoglycan). This can include covalent, ionic, or hydrogen bonding, either directly or indirectly via a linking group such as a divalent linker. The conjugate is typically formed by covalent bonding of the peptide to the glycan through the formation of amide, ester or imino bonds between acid, aldehyde, hydroxy, amino, or hydrazo groups on the respective components of the conjugate. All of these methods are known in the art or are further described in the Examples section of this application or in Hermanson G. T., Bioconjugate Techniques, Academic Press, pp. 169-186 (1996), incorporated herein by reference. The linker typically comprises about 1 to about 30 carbon atoms, more typically about 2 to about 20 carbon atoms. Lower molecular weight linkers (i.e., those having an approximate molecular weight of about 20 to about 500) are typically employed.

In addition, structural modifications of the linker portion of the conjugates are contemplated herein. For example, amino acids may be included in the linker and a number of amino acid substitutions may be made to the linker portion of the conjugate, including but not limited to naturally occurring amino acids, as well as those available from conventional synthetic methods. In another aspect, beta, gamma, and longer chain amino acids may be used in place of one or more alpha amino acids. In another aspect, the linker may be shortened or lengthened, either by changing the number of amino acids included therein, or by including more or fewer beta, gamma, or longer chain amino acids. Similarly, the length and shape of other chemical fragments of the linkers described herein may be modified.

In various embodiments described herein, the linker may include one or more bivalent fragments selected independently in each instance from the group consisting of alkylene, heteroalkylene, cycloalkylene, cycloheteroalkylene, arylene, and heteroarylene each of which is optionally substituted. As used herein heteroalkylene represents a group resulting from the replacement of one or more carbon atoms in a linear or branched alkylene group with an atom independently selected in each instance from the group consisting of oxygen, nitrogen, phosphorus and sulfur.

In various embodiments described herein, a collagen-binding synthetic peptidoglycan may be administered to a patient (e.g., a patient in need of treatment to inhibit platelet activation, such as that involved in thrombosis, platelet binding to exposed collagen of the denuded endothelium, thrombosis, inflammation resulting from denuding the endothelium, intimal hyperplasia, or vasospasm). In various embodiments, the collagen-binding synthetic peptidoglycan can be administered intravenously or into muscle, for example. Suitable routes for parenteral administration include intravascular, intravenous, intraarterial, intramuscular, cutaneous, subcutaneous, percutaneous, intradermal, and intraepidermal delivery. Suitable means for parenteral administration include needle (including microneedle) injectors, infusion techniques, and catheter-based delivery.

In an illustrative embodiment, pharmaceutical formulations for use with collagen-binding synthetic peptidoglycans for parenteral administration or catheter-based delivery comprising: a) a pharmaceutically active amount of the collagen-binding synthetic peptidoglycan; b) a pharmaceutically acceptable pH buffering agent to provide a pH in the range of about pH 4.5 to about pH 9; c) an ionic strength modifying agent in the concentration range of about 0 to about 300 millimolar; and d) water soluble viscosity modifying agent in the concentration range of about 0.25% to about 10% total formula weight or any individual component a), b), c), or d) or any combinations of a), b), c) and d) are provided.

In various embodiments described herein, the pH buffering agents for use in the compositions and methods herein described are those agents known to the skilled artisan and include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or MES.

In various embodiments described herein, the ionic strength modifying agents include those agents known in the art, for example, glycerin, propylene glycol, mannitol, glucose, dextrose, sorbitol, sodium chloride, potassium chloride, and other electrolytes.

Useful viscosity modulating agents include but are not limited to, ionic and non-ionic water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. Typically, non-acidic viscosity enhancing agents, such as a neutral or basic agent are employed in order to facilitate achieving the desired pH of the formulation.

In various embodiments described herein, parenteral formulations may be suitably formulated as a sterile non-aqueous solution or as a dried form to be used in conjunction with a suitable vehicle such as sterile, pyrogen-free water. The preparation of parenteral formulations under sterile conditions, for example, by lyophilisation, may readily be accomplished using standard pharmaceutical techniques well known to those skilled in the art.

In various embodiments described herein, the solubility of a collagen-binding synthetic peptidoglycan used in the preparation of a parenteral formulation may be increased by the use of appropriate formulation techniques, such as the incorporation of solubility-enhancing compositions such as mannitol, ethanol, glycerin, polyethylene glycols, propylene glycol, poloxomers, and others known to those of skill in the art.

In various embodiments described herein, formulations for parenteral administration may be formulated to be for immediate and/or modified release. Modified release formulations include delayed, sustained, pulsed, controlled, targeted and programmed release formulations. Thus, a collagen-binding synthetic peptidoglycan may be formulated as a solid, semi-solid, or thixotropic liquid for administration as an implanted depot providing modified release of the active compound. Illustrative examples of such formulations include drug-coated stents and copolymeric (dl-lactic, glycolic) acid (PGLA) microspheres. In another embodiment, collagen-binding synthetic peptidoglycans or compositions comprising collagen-binding synthetic peptidoglycan may be continuously administered, where appropriate.

In any of the embodiments described herein, the collagen-binding synthetic peptidoglycan can be administered intravascularly into the patient (e.g., into an artery or vein) in any suitable way. In various embodiments described herein, the collagen-binding synthetic peptidoglycan can be administered into a vessel of a patient prior to, during, or after vascular intervention. In various embodiments, vascular interventions, such as percutaneous coronary intervention (PCI), can include, for example, stenting, atherectomy, grafting, and angioplasty, such as balloon angioplasty. Illustratively, the vascular intervention can be one which involves temporarily occluding an artery, such as a coronary artery or a vein (e.g., balloon angioplasty), or it can be one which does not involve temporarily occluding an artery or a vein (e.g., non-balloon angioplasty procedures, stenting procedures that do not involve balloon angioplasty, etc.). Illustrative modes of delivery can include a catheter, parenteral administration, a coating on a balloon, through a porous balloon, a coated stent, and any combinations thereof or any other known methods of delivery of drugs during a vascular intervention procedure. In one illustrative embodiment, the target vessel can include a coronary artery, e.g., any blood vessel which supplies blood to the heart tissue of a patient, including native coronary arteries as well as those which have been grafted into the patient, for example, in an earlier coronary artery bypass procedure.

In any of the embodiments described herein, the target vessel into which the collagen-binding synthetic peptidoglycan is to be administered and on which the vascular intervention procedure is to be performed may contain a blockage, such as a stenosis or some other form of complete or partial blockage which causes reduced blood flow through the vessel. Thus, the collagen-binding synthetic peptidoglycan can be delivered to the vessel via a catheter (e.g., a dilatation catheter, an over-the-wire angioplasty balloon catheter, an infusion catheter, a rapid exchange or monorail catheter, or any other catheter device known in the art) which is percutaneously inserted into the patient and which is threaded through the patient's blood vessels to the target vessel. Various catheter-based devices are known in the art, including those described in U.S. Pat. No. 7,300,454, incorporated herein by reference. In various embodiments described herein where a catheter is used, the catheter used to deliver the collagen-binding synthetic peptidoglycan can be the same catheter through which the vascular intervention is to be performed, or it can be a different catheter (e.g., a different catheter which is percutaneously inserted into the patient via the same or a different cutaneous incision and/or which is threaded through the patient's blood vessels to the target vessel via the same or a different route). In another embodiment, the collagen-binding synthetic peptidoglycan can be injected directly into the target vessel. In another embodiment, the collagen-binding synthetic peptidoglycan can be delivered systemically (i.e., not delivered directly to the target vessel, but delivered by parenteral administration without catheter-based delivery).

In the case where the vessel contains a blockage (e.g., a stenosis), administration can be carried out by delivering the collagen-binding synthetic peptidoglycan directly to the target vessel at the site of the blockage or distal to the blockage or both. In another embodiment, the collagen-binding synthetic peptidoglycan can be delivered to one or more sites proximal to the blockage. Illustratively, the catheter tip can be maintained stationary while the collagen-binding synthetic peptidoglycan is being delivered, or the catheter tip can be moved while the collagen-binding synthetic peptidoglycan is being delivered (e.g., in a proximal direction from a position that is initially distal to the blockage, to or through the blockage, or to a position which is proximal to the blockage).

As indicated above, in one embodiment, the collagen-binding synthetic peptidoglycan can be administered directly into the patient's vessel at a time prior to vascular intervention, e.g., percutaneous coronary intervention. For example, delivery of the collagen-binding synthetic peptidoglycan can be carried out just prior to vascular intervention (e.g., within about 1 hour, such as within about 30 minutes, within about 15 minutes, and/or within about 5 minutes prior to vascular intervention). Optionally, delivery of the collagen-binding synthetic peptidoglycan directly to the target vessel can be continued during all or part of the vascular intervention procedure and/or subsequent to completion of such procedure, or delivery of the collagen-binding synthetic peptidoglycan directly to the target vessel can be stopped prior to the commencement of the vascular intervention procedure and not subsequently recommenced. In any of the embodiments described herein, delivery of the collagen-binding synthetic peptidoglycan can be continuous or it can be effected through a single or multiple administrations. Prior to, during, and/or after the collagen-binding synthetic peptidoglycan is administered to the target vessel, the same collagen-binding synthetic peptidoglycan or one or more different collagen-binding synthetic peptidoglycans can be administered.

In any of the embodiments described herein, the collagen-binding synthetic peptidoglycan can be administered alone or in combination with suitable pharmaceutical carriers or diluents. Diluent or carrier ingredients used in the collagen-binding synthetic peptidoglycan formulation can be selected so that they do not diminish the desired effects of the collagen-binding synthetic peptidoglycan. The collagen-binding synthetic peptidoglycan formulation may be in any suitable form. Examples of suitable dosage forms include aqueous solutions of the collagen-binding peptidoglycan, for example, a solution in isotonic saline, 5% glucose or other well-known pharmaceutically acceptable liquid carriers such as alcohols, glycols, esters and amides.

Suitable dosages of the collagen-binding synthetic peptidoglycan can be determined by standard methods, for example by establishing dose-response curves in laboratory animal models or in clinical trials. Illustratively, suitable dosages of collagen-binding synthetic peptidoglycan (administered in a single bolus or over time) include from 1 ng/kg to about 10 mg/kg, 100 ng/kg to about 1 mg/kg, from about 1 μg/kg to about 500 μg/kg, or from about 100 μg/kg to about 400 μg/kg. In each of these embodiments, dose/kg refers to the dose per kilogram of patient mass or body weight. In other illustrative aspects, effective doses can range from about 0.01 μg to about 1000 mg per dose, 1 μg to about 100 mg per dose, or from about 100 μg to about 50 mg per dose, or from about 500 μg to about 10 mg per dose or from about 1 mg to 10 mg per dose, or from about 1 to about 100 mg per dose, or from about 1 mg to 5000 mg per dose, or from about 1 mg to 3000 mg per dose, or from about 100 mg to 3000 mg per dose, or from about 1000 mg to 3000 mg per dose.

Vascular intervention, such as percutaneous coronary intervention, can be carried out by any conventional procedure prior to, during, or after administration of the collagen-binding synthetic peptidoglycan. Examples of vascular intervention procedures contemplated for use in conjunction with the method of the present invention include stenting, atherectomy, and angioplasty, such as balloon angioplasty. The vascular intervention procedure can be one which involves temporarily occluding the vessel (e.g., balloon angioplasty), or it can be one which does not involve temporarily occluding the vessel (e.g., non-balloon angioplasty procedures, stenting procedures that do not involve balloon angioplasty, etc.). Illustrative modes of delivery can include a catheter, parenteral administration, a coating on a balloon, through a porous balloon, a coated stent, and any combinations thereof or any other known methods of delivery of drugs during a vascular intervention procedure.

In any of the embodiments herein described, kits for carrying out vascular intervention, such as the kits described above, are contemplated. The kits can include a catheter or a stent and a collagen-binding synthetic peptidoglycan. The collagen-binding synthetic peptidoglycan can be provided in any of the formulations discussed above and in an amount needed to carry out a single vascular intervention, such as from 1 ng/kg to about 10 mg/kg, 100 ng/kg to about 1 mg/kg, from about 1 μg/kg to about 500 μg/kg, or from about 100 μg/kg to about 400 μg/kg. In each of these embodiments, dose/kg refers to the dose per kilogram of patient mass or body weight. In various embodiments herein described, effective doses provided in the formulations can range from about 0.01 μg to about 1000 mg per dose, 1 μg to about 100 mg per dose, or from about 100 μg to about 50 mg per dose, or from about 500 μg to about 10 mg per dose or from about 1 mg to 10 mg per dose, or from about 1 to about 100 mg per dose, or from about 1 mg to 5000 mg per dose, or from about 1 mg to 3000 mg per dose, or from about 100 mg to 3000 mg per dose, or from about 1000 mg to 3000 mg per dose. Articles of manufacture are also contemplated for any of these embodiments.

In any of the kit or article of manufacture embodiments described herein, the kit or article of manufacture can comprise a dose or multiple doses of the collagen-binding synthetic peptidoglycan. The collagen-binding synthetic peptidoglycan can be in a primary container, for example, a glass vial, such as an amber glass vial with a rubber stopper and/or an aluminum tear-off seal. In another embodiment, the primary container can be plastic or aluminum, and the primary container can be sealed. In another embodiment, the primary container may be contained within a secondary container to further protect the composition from light.

In any of the embodiments described herein, the kit or article of manufacture can contain instructions for use. Other suitable kit or article of manufacture components include excipients, disintegrants, binders, salts, local anesthetics (e.g., lidocaine), diluents, preservatives, chelating agents, buffers, tonicity agents, antiseptic agents, wetting agents, emulsifiers, dispersants, stabilizers, and the like. These components may be available separately or admixed with the collagen-binding synthetic peptidoglycan. Any of the composition embodiments described herein can be used to formulate the kit or article of manufacture.

In various embodiments herein described, the kit can contain more than one catheter or a stent and a plurality of separate containers, each containing sterilized collagen-binding synthetic peptidoglycan formulations in an amount needed to carry out a single or multiple vascular interventions. Any type of stent or catheter may be included with the kit, including, for example, dilatation catheters, over-the-wire angioplasty balloon catheters, infusion catheters, rapid exchange or monorail catheters, and the like.

It is also contemplated that any of the formulations described herein may be used to administer the collagen-binding synthetic peptidoglycan (e.g., one or more types) either in the absence or the presence of a catheter-based device. The collagen-binding synthetic proteoglycan can be formulated in an excipient. In any of the embodiments described herein, the excipient can have a concentration ranging from about 0.4 mg/ml to about 6 mg/ml. In various embodiments, the concentration of the excipient may range from about 0.5 mg/ml to about 10 mg/ml, about 0.1 mg/ml to about 6 mg/ml, about 0.5 mg/ml to about 3 mg/ml, about 1 mg/ml to about 3 mg/ml, about 0.01 mg/ml to about 10 mg/ml, and about 2 mg/ml to about 4 mg/ml.

In various embodiments described herein, the dosage of the collagen-binding synthetic peptidoglycan, can vary significantly depending on the patient condition, the disease state being treated, the route of administration and tissue distribution, and the possibility of co-usage of other therapeutic treatments. The effective amount to be administered to a patient is based on body surface area, patient weight or mass, and physician assessment of patient condition. In various exemplary embodiments, an effective dose can range from about 1 ng/kg to about 10 mg/kg, 100 ng/kg to about 1 mg/kg, from about 1 μg/kg to about 500 μg/kg, or from about 100 μg/kg to about 400 μg/kg. In each of these embodiments, dose/kg refers to the dose per kilogram of patient mass or body weight. In other illustrative aspects, effective doses can range from about 0.01 μg to about 1000 mg per dose, 1 μg to about 100 mg per dose, or from about 100 μg to about 50 mg per dose, or from about 500 μg to about 10 mg per dose or from about 1 mg to 10 mg per dose, or from about 1 to about 100 mg per dose, or from about 1 mg to 5000 mg per dose, or from about 1 mg to 3000 mg per dose, or from about 100 mg to 3000 mg per dose, or from about 1000 mg to 3000 mg per dose. In any of the various embodiments described herein, effective doses can range from about 0.01 μg to about 1000 mg per dose, 1 μg to about 100 mg per dose, about 100 μg to about 1.0 mg, about 50 μg to about 600 μg, about 50 μg to about 700 μg, about 100 μg to about 200 μg, about 100 μg to about 600 μg, about 100 μg to about 500 μg, about 200 μg to about 600 μg, or from about 100 μg to about 50 μg per dose, or from about 500 μg to about 10 mg per dose or from about 1 mg to 10 mg per dose. In other illustrative embodiments, effective doses can be 1 μg, 10 μg, 25 μg, 50 μg, 75 μg, 100 μg, 125 μg, 150 μg, 200 μg, 250 μg, 275 μg, 300 μg, 350 μg, 400 μg, 450 μg, 500 μg, 550 μg, 575 μg, 600 μg, 625 μg, 650 μg, 675 μg, 700 μg, 800 μg, 900 μg, 1.0 mg, 1.5 mg, 2.0 mg, 10 mg, 100 mg, or 100 mg to 30 grams.

Any effective regimen for administering the collagen-binding synthetic peptidoglycan can be used. For example, the collagen-binding synthetic peptidoglycan can be administered as a single dose, or as a multiple-dose daily regimen. Further, a staggered regimen, for example, one to five days per week can be used as an alternative to daily treatment.

In various embodiments described herein, the patient is treated with multiple injections of the collagen-binding synthetic peptidoglycan. In one embodiment, the patient is injected multiple times (e.g., about 2 up to about 50 times) with the collagen-binding synthetic peptidoglycan, for example, at 12-72 hour intervals or at 48-72 hour intervals. Additional injections of the collagen-binding synthetic peptidoglycan can be administered to the patient at an interval of days or months after the initial injections(s).

In any of the embodiments herein described, it is to be understood that a combination of two or more collagen-binding synthetic peptidoglycans, differing in the peptide portion, the glycan portion, or both, can be used in place of a single collagen-binding synthetic peptidoglycan.

It is also appreciated that in the foregoing embodiments, certain aspects of the compounds, compositions and methods are presented in the alternative in lists, such as, illustratively, selections for any one or more of G and P. It is therefore to be understood that various alternate embodiments of the invention include individual members of those lists, as well as the various subsets of those lists. Each of those combinations are to be understood to be described herein by way of the lists.

In the following illustrative examples, the terms “synthetic peptidoglycan” and “conjugate” are used synonymously with the term “collagen-binding synthetic peptidoglycan.”

### Example 1

### Peptide Synthesis

All peptides were synthesized using a Symphony peptide synthesizer (Protein Technologies, Tucson, Ariz.), utilizing an FMOC protocol on a Knorr resin. The crude peptide was released from the resin with TFA and purified by reverse phase chromatography on an AKTAexplorer (GE Healthcare, Piscataway, N.J.) utilizing a Grace-Vydac 218TP C-18 reverse phase column and a gradient of water/acetonitrile 0.1% TFA. Dansyl-modified peptides were prepared by adding an additional coupling step with dansyl-Gly (Sigma) before release from the resin. Peptide structures were confirmed by mass spectrometry. The following peptides were prepared as described above: RRANAALKAGELYKSILYGC (SEQ ID NO: 1), SYIRIADTNIT (SEQ ID NO: 10), Dansyl-GRRANAALKAGELYKSILYGC (SEQ ID NO: 28), and Dansyl-GSYIRIADTNIT (SEQ ID NO: 29). These peptides are abbreviated SILY, SYIR, Z-SILY, and Z-SYIR. A biotin-labeled Z-SYIR peptide has also been synthesized using protocols known in the art and the peptide is amide terminated. Additional peptides, KELNLVYTGC (abbreviated KELN) (SEQ ID NO: 12) and GSITTIDVPWNVGC (abbreviated GSIT) (SEQ ID NO: 16) were prepared as described above or purchased (GenScript, Piscataway, N.J.).

### Example 2

### Conjugation of SILY to Dermatan Sulfate

PDPH Attachment to oxDS

The bifunctional crosslinker PDPH (Pierce), reactive to sulfhydryl and amine groups, was used to conjugate SILY to oxDS. In the first step of the reaction, oxDS was dissolved in coupling buffer (0.1 M sodium phosphate, 0.25 M sodium chloride, pH 7.2) to a final concentration of 1.2 mM. PDPH was added in 10-fold molar excess, and the reaction proceeded at room temperature for 2 hours. Excess PDPH (MW 229 Da) was separated by gel filtration on an Akta Purifier using an XK 26-40 column packed with Sephadex G-25 medium and equilibrated with MilliQ water. Eluent was monitored at 215 nm, 254 nm, and 280 nm. The first eluting peak containing DS-PDPH was collected and lyophilized for conjugating with SILY.

### Conjugation of SILY

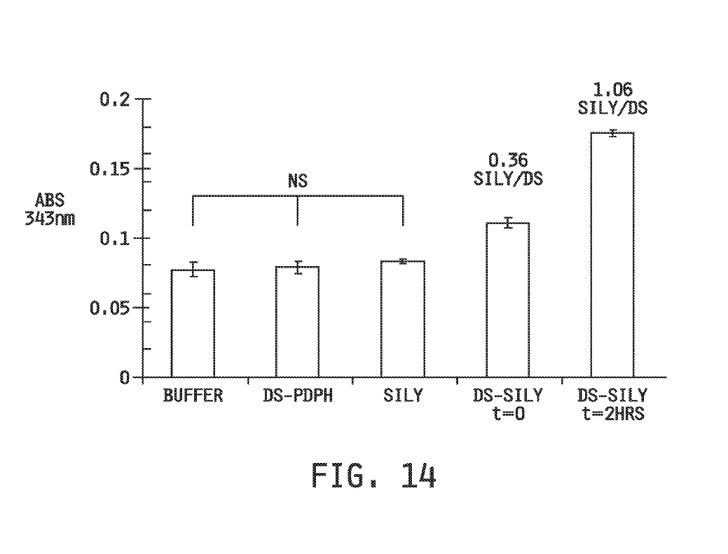

The peptide was dissolved in a 5:1 molar excess in coupling buffer at a final peptide concentration of approximately 1 mM (limited by peptide solubility). The reaction was allowed to proceed at room temperature overnight, and excess peptide was separated and the DS-SILY conjugate isolated by gel filtration as described above. See FIG. 14 showing a SILY/DS ratio of 1.06 after coupling.

### Example 3

### Conjugation of Z-SILY to Dermatan Sulfate

Dermatan sulfate was conjugated to Z-SILY according to the method of EXAMPLE 2.

### Example 4

### Conjugation of KELN to Dermatan Sulfate

Dermatan sulfate was conjugated to KELN according to the method of EXAMPLE 2.

### Example 5

### Conjugation of GSIT to Dermatan Sulfate

Dermatan sulfate was conjugated to GSIT according to the method of EXAMPLE 2.

### Example 6

### Conjugation of Z-SYIR to Dermatan Sulfate

Dermatan sulfate was conjugated to Z-SYIR using a method similar to that described in EXAMPLE 2.

### Example 7

### Conjugation of SILY to Heparin

Oxidized Heparin (oxHep) (MW=19.7 kDa) containing 1 aldehyde per molecule (purchased from Celsus Laboratories, Cincinnati, Ohio). Additional aldehydes were formed by further oxidation in sodium meta-periodate as follows. oxHep was dissolved in 0.1 M sodium acetate pH 5.5 at a concentration of 10 mg/mL. Sodium meta-periodate was then added at a concentration of 2 mg/mL and allowed to react for 4 hours at room temperature protected from light. Excess sodium meta-periodate was removed by desalting using a HiTrap size exclusion column (GE Healthcare) and oxHep was lyophilized protected from light until conjugation with PDPH.

oxHep was conjugated to PDPH by the method described for DS-PDPH conjugation, EXAMPLE 2. PDPH was reacted in 50-fold molar excess. To achieve a higher PDPH concentration, 10 mg PDPH was dissolved in 75 μL DMSO and mixed with 1 mL coupling buffer containing oxHep. The reaction proceeded at room temperature for 2.5 hours and excess PDPH was removed by desalting. Heparin containing PDPH (Hep-PDPH) was stored as a lyophilized powder until reacted with SILY.

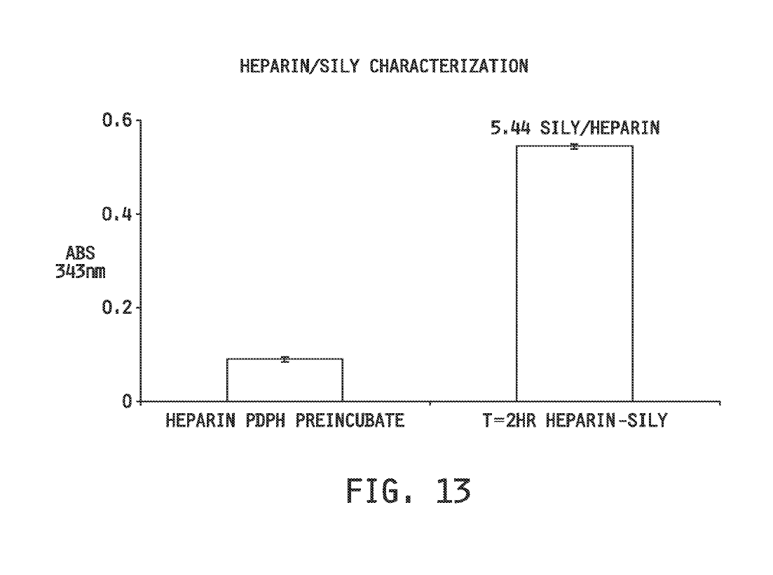

SILY was reacted in 10-fold molar excess with Hep-PDPH as described for DS-SILY conjugation in EXAMPLE 2. The reaction was monitored as described for DS-SILY in EXAMPLE 2 and showed 5.44 SILY peptides conjugated per heparin molecule as shown in FIG. 13.

### Example 8

### Conjugation of GSIT to Heparin

Heparin was conjugated to GSIT according to the method of EXAMPLE 7 (abbreviated Hep-GSIT).

### Example 9

### Conjugation of SILY to Dextran

Dextran was conjugated to SILY according to the method of EXAMPLE 7 replacing heparin with dextran. Modification of the conditions for oxidation of dextran with sodium meta-periodate in the first step to allowed preparation of conjugates with different molar ratios of SILY to dextran. For example dextran-SILY conjugates with a molar ratio of SILY to dextran of about 6 and a dextran-SILY conjugate with a molar ratio of SILY to dextran of about 9 were prepared (abbreviated Dex-SILY6 and Dex-SILY9).

### Example 10

### Conjugation of SILY to Hyaluronan

Hyaluronan was conjugated to SILY according to the method of EXAMPLE 7 (abbreviated HA-SILY).

### Example 11

### SILY Binding to Collagen (Biacore)

Biacore studies were performed on a Biacore 2000 using a CM-3 chip (Biacore, Inc., Piscataway, N.J.). The CM-3 chip is coated with covalently attached carboxymethylated dextran, which allows for attachment of the substrate collagen via free amine groups. Flow cells (FCs) 1 and 2 were used, with FC-1 as the reference cell and FC-2 as the collagen immobilized cell. Each FC was activated with EDC-NHS, and 1500 RU of collagen was immobilized on FC-2 by flowing 1 mg/mL collagen in sodium acetate, pH 4, buffer at 5 μL/min for 10 min. Unreacted NETS-ester sites were capped with ethanolamine; the control FC-1 was activated and capped with ethanolamin.

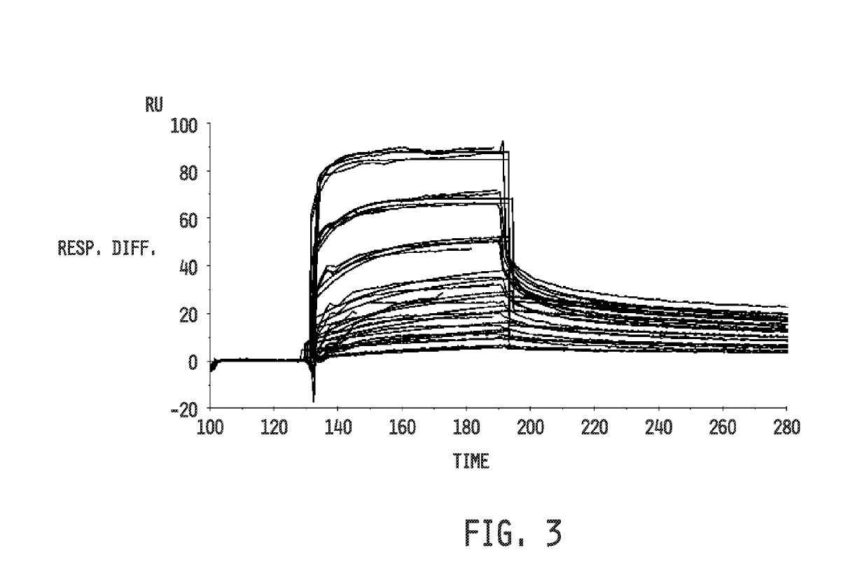

To determine peptide binding affinity, SILY was dissolved in 1×HBS-EP buffer (Biacore) at varying concentrations from 100 μM to 1.5 μm in 2-fold dilutions. The flow rate was held at 90 μL/min which is in the range suggested by Myska for determining binding kinetics (Myska, 1997). The first 10 injections were buffer injections, which help to prime the system, followed by randomized sample injections, run in triplicate. Analysis was performed using BIAevaluation software (Biacore). Representative association/disassociation curves are shown in FIG. 3 demonstrating that the SILY peptide binds reversibly with collagen. K_{D}=1.2 was calculated from the on-off binding kinetics.

### Example 12

### Z-SILY Binding to Collagen

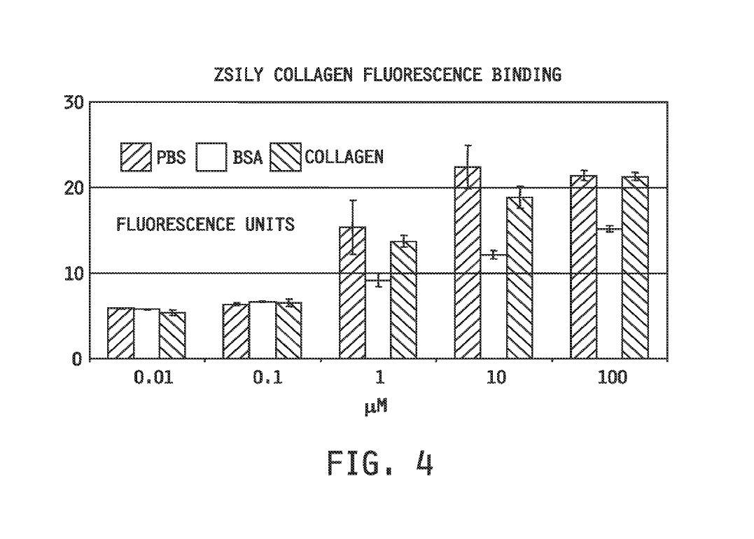

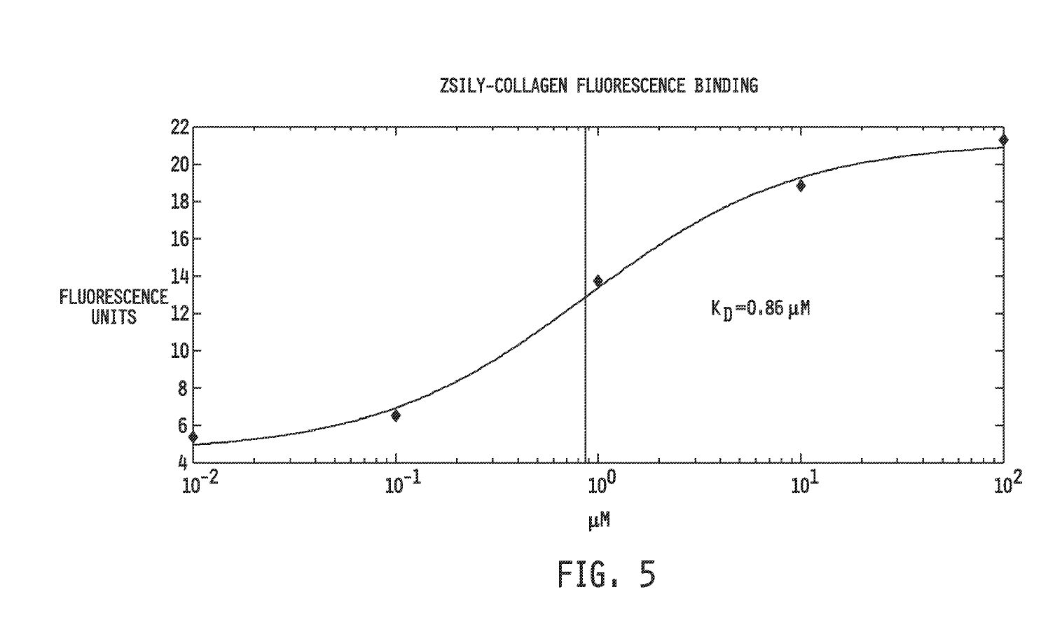

Binding assays were done in a 96-well high-binding plate, black with a clear bottom (Costar). Collagen was compared to untreated wells and BSA coated wells. Collagen and BSA were immobilized at 37° C. for 1 hr by incubating 90 μL/well at concentrations of 2 mg/mL in 10 mM HCl and 1×PBS, respectively. Each well was washed 3× with 1×PBS after incubating. Z-SILY was dissolved in 1×PBS at concentrations from 100 μM to 10 nM in 10-fold dilutions. Wells were incubated for 30 min at 37° C. and rinsed 3× with PBS and then filled with 90 μL of 1×PBS. Fluorescence readings were taken on an M5 Spectramax Spectrophotometer (Molecular Devices) at excitation/emission wavelengths of 335 nm/490 nm respectively. The results are shown in FIGS. 4 and 5. K_{D}=0.86 μM was calculated from the equilibrium kinetics.

### Example 13

### Characterizing DS-SILY

To determine the number of SILY molecules conjugated to DS, the production of pyridine-2-thione was measured using a modified protocol provided by Pierce. Dermatan sulfate with 1.1 PDPH molecules attached was dissolved in coupling buffer (0.1 M sodium phosphate, 0.25 M sodium chloride) at a concentration of 0.44 mg/mL and absorbance at 343 nm was measured using a SpectraMax M5 (Molecular Devices). SILY was reacted in 5-fold molar excess and absorbance measurements were repeated immediately after addition of SILY and after allowing to react for 2 hours. To be sure SILY does not itself absorb at 343 nm, coupling buffer containing 0.15 mg/mL SILY was measured and was compared to absorbance of buffer alone.

The number of SILY molecules conjugated to DS was calculated by the extinction coefficient of pyridine-2-thione using the following equation (Abs₃₄₃/8080)×(MW_{DS}/DS_{mg/mL}). The results are shown in FIG. 14.

### Example 14

### Collagen Binding, Fluorescence Data—DS-SILY

In order to determine whether the peptide conjugate maintained its ability to bind to collagen after its conjugation to DS, a fluorescent binding assay was performed. A fluorescently labeled version of SILY, Z-SILY, was synthesized by adding dansylglycine to the amine terminus. This peptide was conjugated to DS and purified using the same methods described for SILY.

Binding assays were done in a 96-well high binding plate, black with a clear bottom (Costar). Collagen was compared to untreated wells and BSA coated wells. Monomeric collagen (Advanced Biomatrix Cat. No. 5010) and BSA were immobilized at 37° C. for 1 hr by incubating 90 μL/well at concentrations of 2 mg/mL in 10 mM HCl and 1×PBS respectively. Each well was washed 3× with 1×PBS after incubating.

Wells were preincubated with DS at 37° C. for 30 min to eliminate nonspecific binding of DS to collagen. Wells were rinsed 3× with 1×PBS before incubating with DS-Z-SILY. DS-Z-SILY was dissolved in 1×PBS at concentrations from 100 μM to 10 nM in 10-fold dilutions. Wells were incubated for 30 min at 37° C. and rinsed 3× and then filled with 90 μL of 1×PBS. Fluorescence readings were taken on an M5 Spectramax Spectrophotometer (Molecular Devices) at excitation/emission wavelengths of 335 nm/490 nm, respectively.

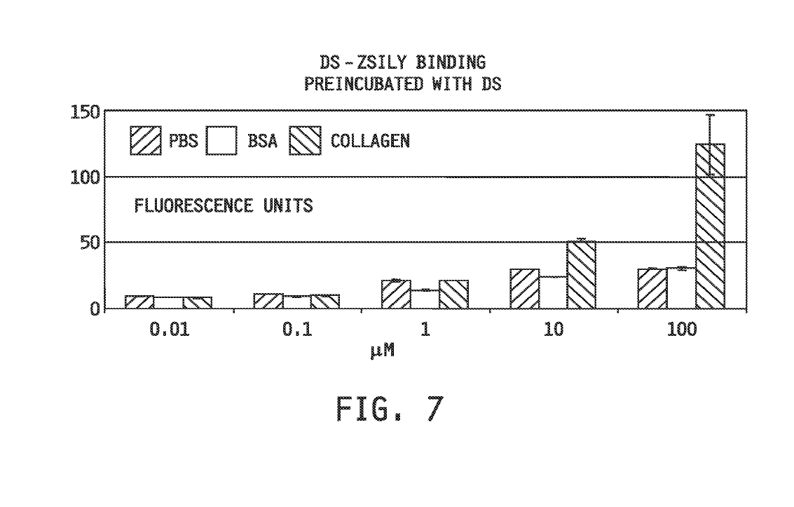

Fluorescence binding of DS-Z-SILY on immobilized collagen, BSA, and untreated wells are compared in FIG. 7. Results show that DS-Z-SILY binds specifically to the collagen-treated wells over BSA and untreated wells. The untreated wells of the high bind plate were designed to be a positive control, though little binding was observed relative to collagen treated wells. These results suggest that SILY maintains its ability to bind to collagen after it is conjugated to DS. Preincubating with DS did not prevent binding, suggesting that the conjugate binds separately from DS alone.

### Example 15

### Preparation of Type I Collagen Gels

Gels were made with Nutragen collagen (Inamed, Freemont, Calif.) at a final concentration of 4 mg/mL collagen. Nutragen stock is 6.4 mg/mL in 10 mM HCl. Gel preparation was performed on ice, and fresh samples were made before each test. The collagen solution was adjusted to physiologic pH and salt concentration, by adding appropriate volumes of 10×PBS (phosphate buffered saline), 1×PBS, and 1 M NaOH. For most experiments, samples of DS, decorin, DS-SILY, or DS-SYIR were added at a 10:1 collagen:sample molar ratio by a final 1×PBS addition (equal volumes across treatments) in which the test samples were dissolved at appropriate concentrations. In this way, samples are constantly kept at pH 7.4 and physiologic salt concentration. Collagen-alone samples received a 1×PBS addition with no sample dissolved. Fibrillogenesis will be induced by incubating neutralized collagen solutions at 37° C. overnight in a humidified chamber to avoid dehydration. Gel solutions with collagen:sample molar ratios of other than 10:1 were prepared similarly.

### Example 16

### Viscoelastic Characterization of Collagen Type III Containing Gels

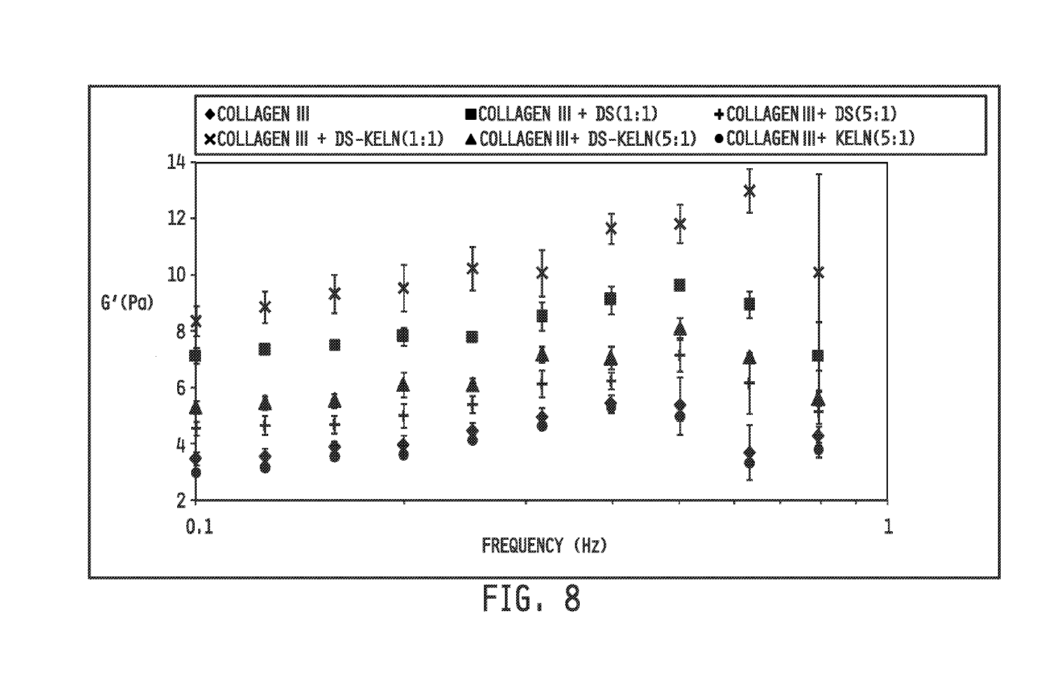

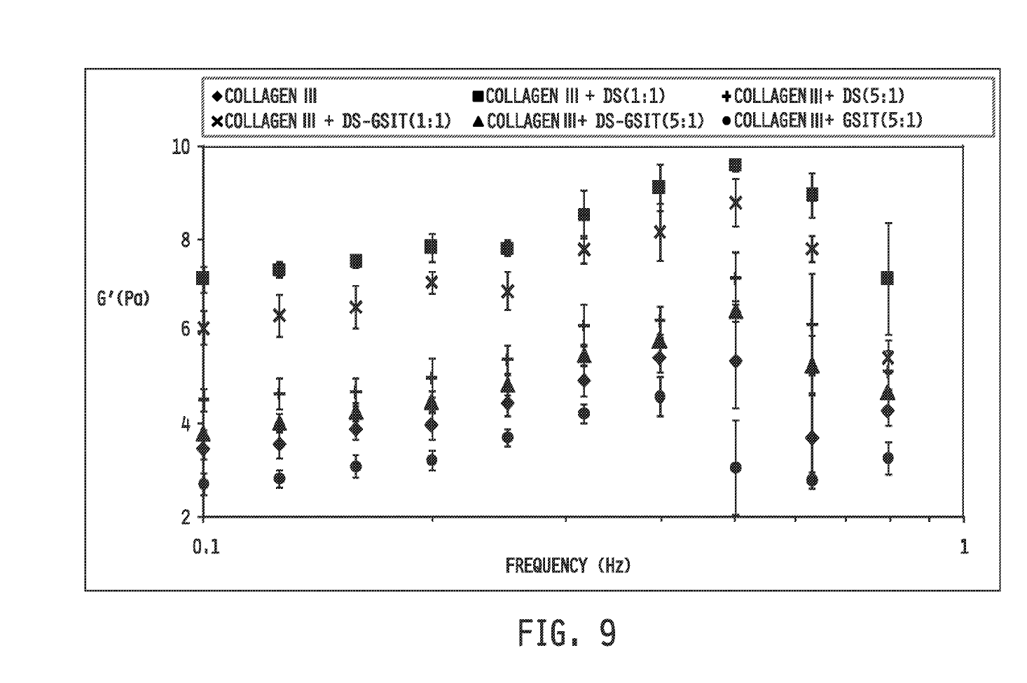

Gels containing type III collagen were prepared as in EXAMPLE 15 with the following modifications: treated and untreated gel solutions were prepared using a collagen concentration of 1.5 mg/mL (90% collagen type III (Millipore), 10% collagen type I), 200 samples were pipetted onto 20 mm diameter wettable surfaces of hydrophobic printed slides. These solutions were allowed to gel at 37° C. for 24 hours. Gels were formed from collagen alone, collagen treated with dermatan sulfate (1:1 and 5:1 molar ratio), and collagen treated with the collagen III-binding peptides alone (GSIT and KELN, 5:1 molar ratio) served as controls. The treated gels contained the peptidoglycans (DS-GSIT or DS-KELN at 1:1 and 5:1 molar ratios. All ratios are collagen:treatment compound ratios. The gels were characterized as in EXAMPLE 18, except the samples were tested over a frequency range from 0.1 Hz to 1.0 Hz at a controlled stress of 1.0 Pa. As shown in FIGS. 8 and 9, the dermatan sulfate-GSIT conjugate and the dermatan sulfate-KELN conjugate (synthetic peptidoglycans) can influence the viscoelastic properties of gels formed with collagen type III.

### Example 17

### Fibrillogenesis

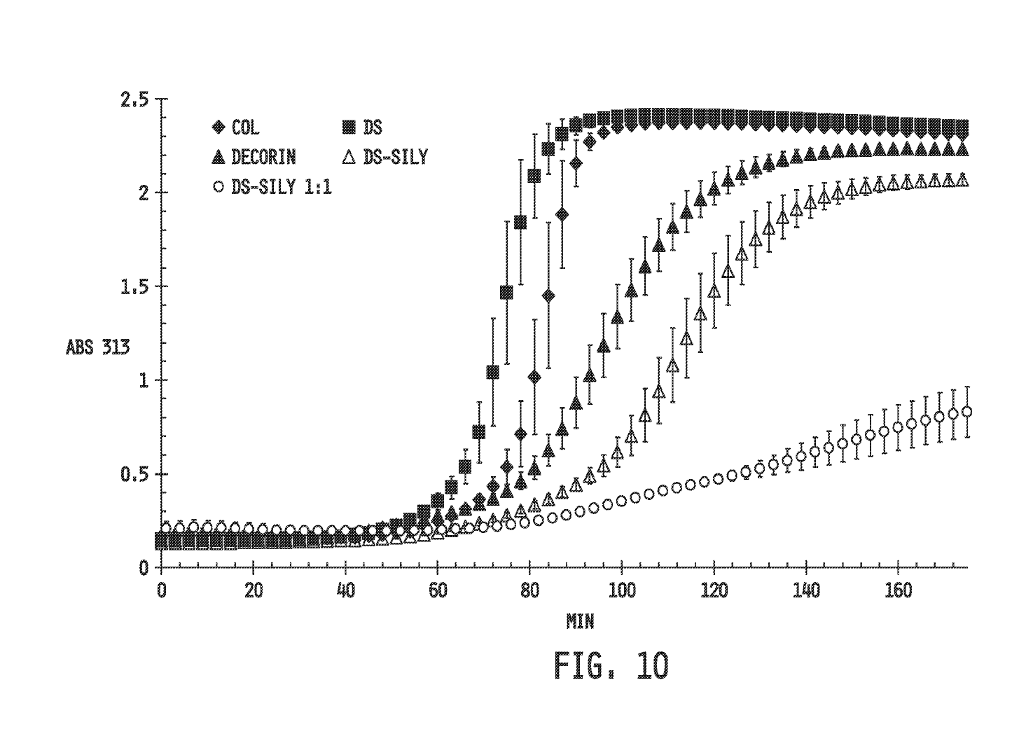

Collagen fibrillogenesis was monitored by measuring turbidity related absorbance at 313 nm providing information on rate of fibrillogenesis and fibril diameter. Gel solutions were prepared as described in EXAMPLE 15 (4 mg/mL collagen, 10:1 collagen:treatment, unless otherwise indicated) and 50 uL/well were added at 4° C. to a 384-well plate. The plate was kept at 4° C. for 4 hours before initiating fibril formation. A SpectraMax M5 at 37° C. was used to measure absorbance at 313 nm at 30 s intervals for 6 hours. The results are shown in FIG. 10. Dermatan sulfate-SILY decreases the rate of fibrillogenesis.

### Example 18

### Cryo-SEM Measurements on Collagen Type III

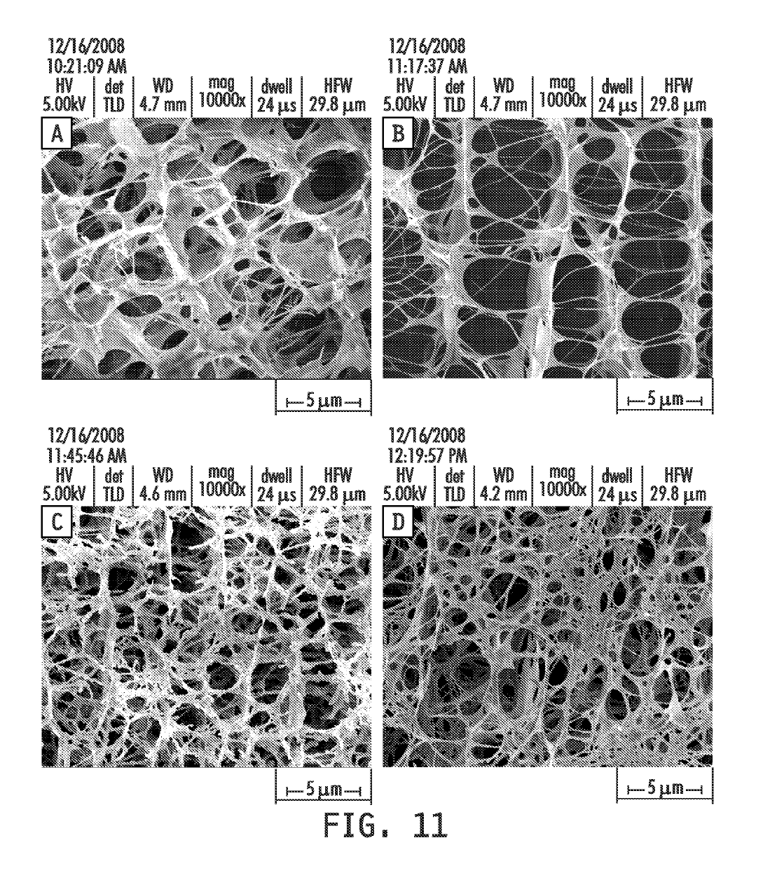

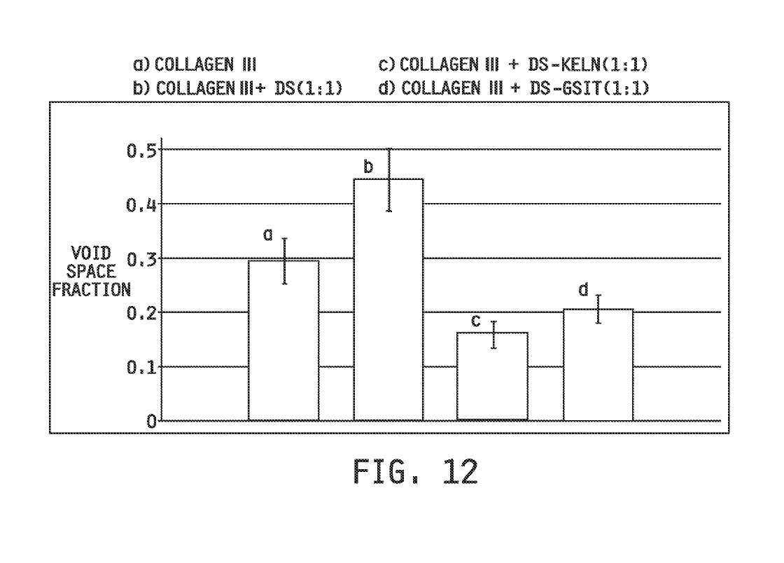

Gels for cryo-SEM were formed, as in EXAMPLE 15, directly on the SEM stage and incubated at 37° C. overnight with the following modifications. The collagen concentration was 1 mg/mL (90% collagen type III, 10% collagen type I). The collagen:DS ratio was 1:1 and the collagen:peptidoglycan ratio was 1:1. The images were recorded as in EXAMPLE 19. The ratio of void volume to fibril volume was measured using a variation of the method in EXAMPLE 28. The results are shown in FIGS. 11 and 12. Dermatan sulfate-KELN and dermatan sulfate-GSIT decrease void space (increase fibril diameter and branching) in the treated collagen gels.

### Example 19

### AFM Confirmation of D-Banding

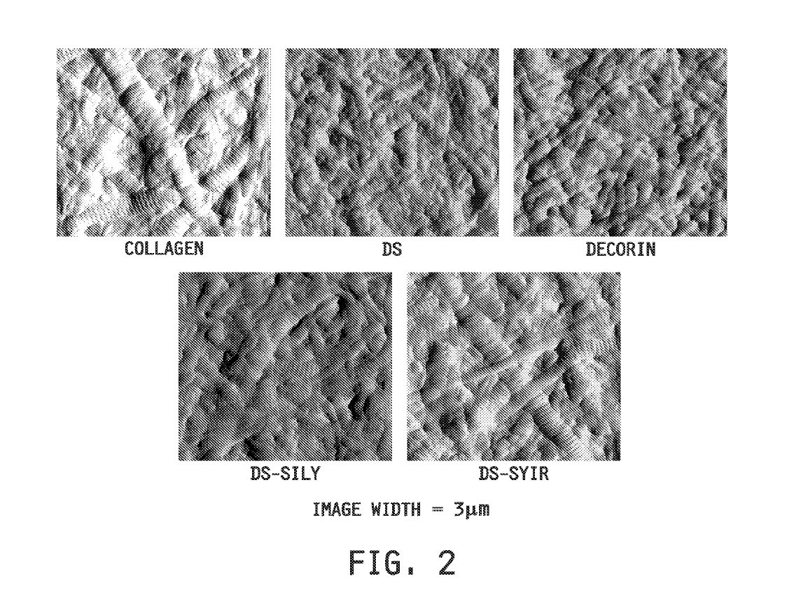

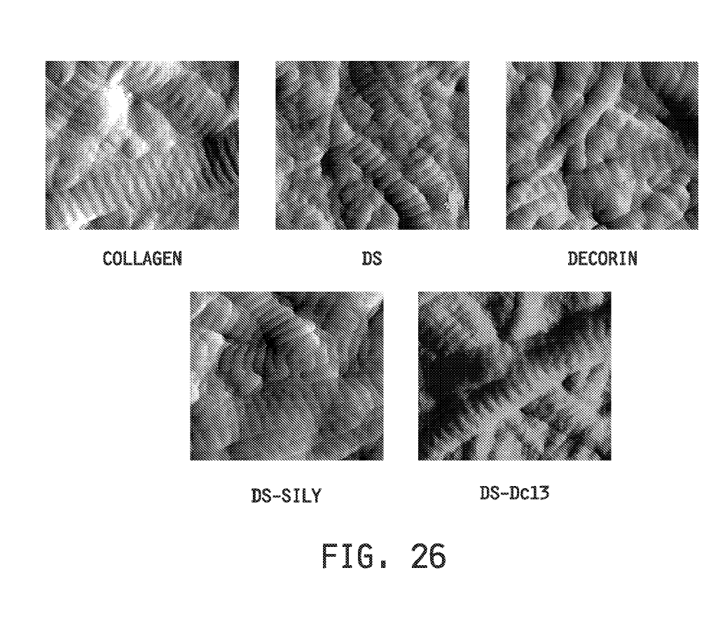

Gel solutions were prepared as described in EXAMPLE 15 and 20 μL of each sample were pipetted onto a glass coverslip and allowed to gel overnight in a humidified incubator. Gels were dehydrated by treatment with graded ethanol solutions (35%, 70%, 85%, 95%, 100%), 10 min in each solution. AFM images were made in contact mode, with a scan rate of 2 Hz (Multimode SPM, Veeco Instruments, Santa Barbara, Calif., USA, AFM tips Silicon Nitride contact mode tip k=0.05 N/m, Veeco Instruments) Deflection setpoint: 0-1 Volts. D-banding was confirmed in all treatments as shown in FIGS. 2 and 26.

### Example 20

### Collagen Remodeling

### Tissue Sample Preparation

Following a method by Grassl, et al. (Grassl, et al., Journal of Biomedical Materials Research 2002, 60, (4), 607-612), which is herein incorporated in its entirety, collagen gels with or without synthetic PG mimics were formed as described in EXAMPLE 15. Human aortic smooth muscle cells (Cascade Biologics, Portland, Oreg.) were seeded within collagen gels by adding 4×10⁶ cells/mL to the neutralized collagen solution prior to incubation. The cell-collagen solutions were pipetted into an 8-well Lab-Tek chamber slide and incubated in a humidified 37° C. and 5% CO₂ incubator. After gelation, the cell-collagen gels will be covered with 1 mL Medium 231 as prescribed by Cascade. Every 3-4 days, the medium was removed from the samples and the hydroxyproline content measured by a standard hydroxyproline assay (Reddy, 1996).

### Hydroxyproline Content

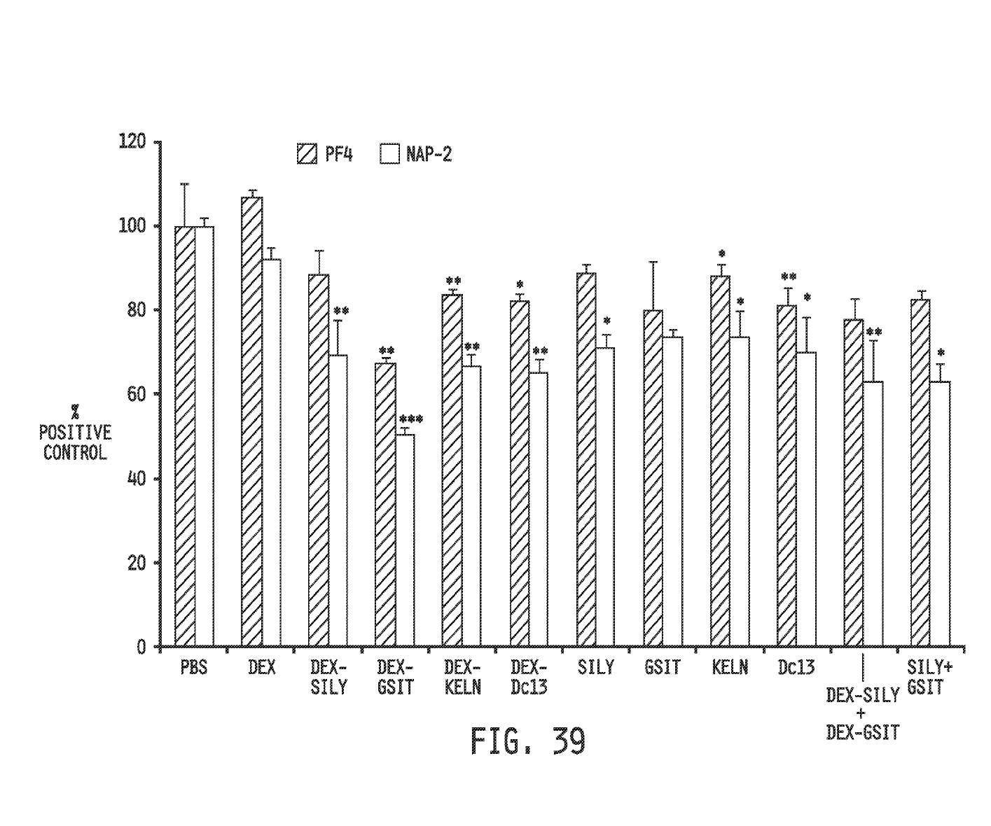

To measure degraded collagen in the supernatant medium, the sample was lyophilized, the sample hydrolyzed in 2 M NaOH at 120° C. for 20 min. After cooling, free hydroxyproline was oxidized by adding chloramine-T (Sigma) and reacting for 25 min at room temperature. Ehrlich's aldehyde reagent (Sigma) was added and allowed to react for 20 min at 65° C. and followed by reading the absorbance at 550 nm on an M-5 spectrophotometer (Molecular Devices). Hydroxyproline content in the medium is an indirect measure degraded collagen and tissue remodeling potential. Cultures were incubated for up to 30 days and three samples of each treatment measured. Gels incubated without added cells were used as a control. Free peptides SILY and Dc 13 resulted in greater collagen degradation compared to collagen alone as measured by hydroxyproline content in cell medium as shown in FIG. 39.

### Cell Viability

Cell viability was determined using a live/dead violet viability/vitality kit (Molecular Probes. The kit contains calcein-violet stain (live cells) and aqua-fluorescent reactive dye (dead cells). Samples were washed with 1×PBS and incubated with 300 μL of dye solution for 1 hr at room temperature. To remove unbound dye, samples were rinsed with 1×PBS. Live and dead cells were counted after imaging a 2-D slice with filters 400/452 and 367/526 on an Olympus FV1000 confocal microscope with a 20× objective. Gels were scanned for representative regions and 3 image sets were taken at equal distances into the gel for all samples.

### Example 21

### Preparation of DS-Dc 13

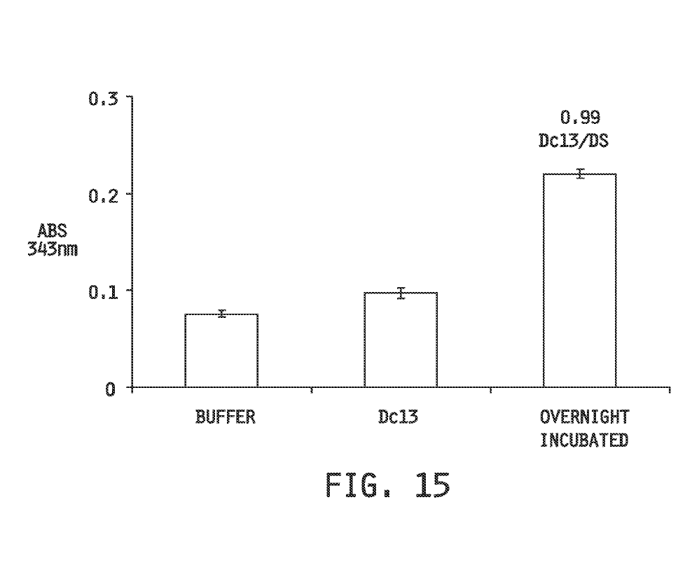

The Dc13 peptide sequence is SYIRIADTNITGC (SEQ ID NO: 9) and its fluorescently labeled form is ZSYIRIADTNITGC (SEQ ID NO: 30), where Z designates dansylglycine. Conjugation to dermatan sulfate using the heterobifunctional crosslinker PDPH is performed as described for DS-SILY in EXAMPLE 2. As shown in FIG. 15, the molar ratio of Dc13 to dermatan sulfate in the conjugate (DS-Dc13) was about 1.

### Example 22

### Fluorescence Binding Assay for DS-ZSILY

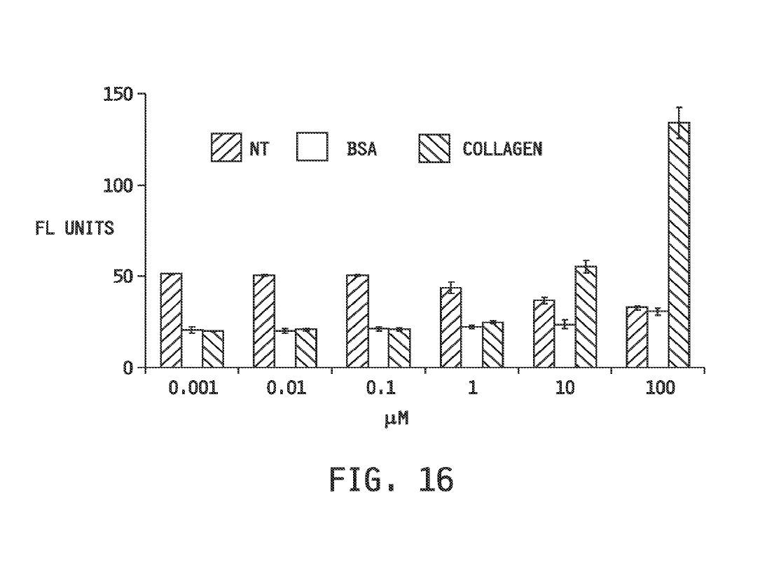

The fluorescence binding assays described for DS-ZSILY was performed with peptide sequence ZSYIRIADTNITGC (ZDc13) (SEQ ID NO: 30). The results appear in FIG. 16, showing that DS-ZDc13 binds specifically to the collagen surface in a dose-dependent manner, though saturation was not achieved at the highest rate tested.

### Example 23

### Fibrillogenesis Assay for DS-Dc13

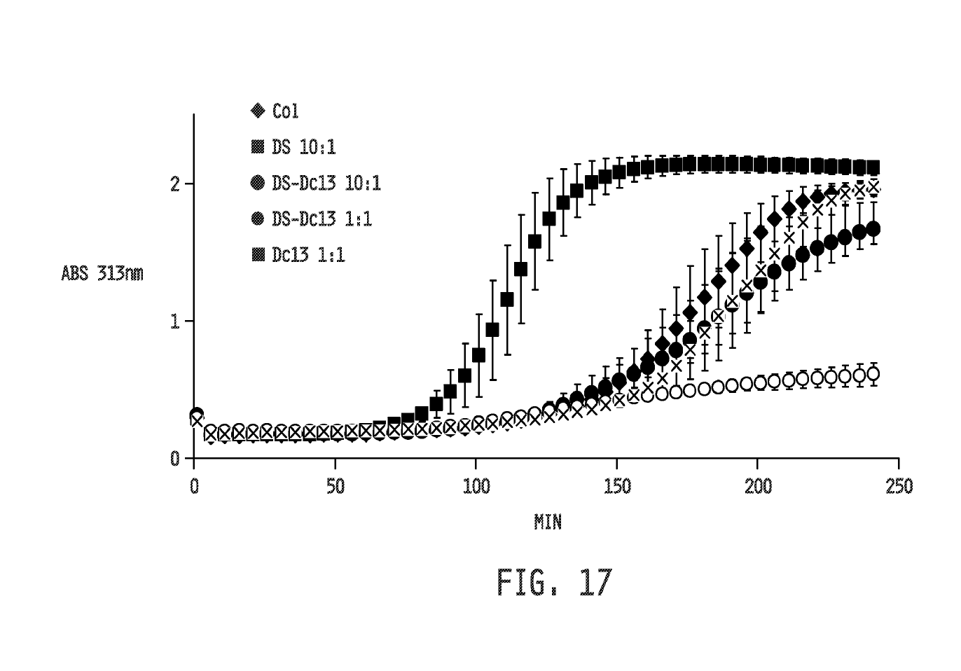

A fibrillogenesis assay as described for DS-SILY, EXAMPLE 17, performed with the conjugate DS-Dc13. The results shown in FIG. 17 indicate that the DS-Dc13 delays fibrillogenesis and decreases overall absorbance in a dose-dependent manner. Free Dc13 peptide in contrast has little effect on fibrillogenesis compared to collagen alone at the high 1:1 collagen:additive molar ratio.

### Example 24

### Use of Cryo-SEM to Measure Fibril Diameters

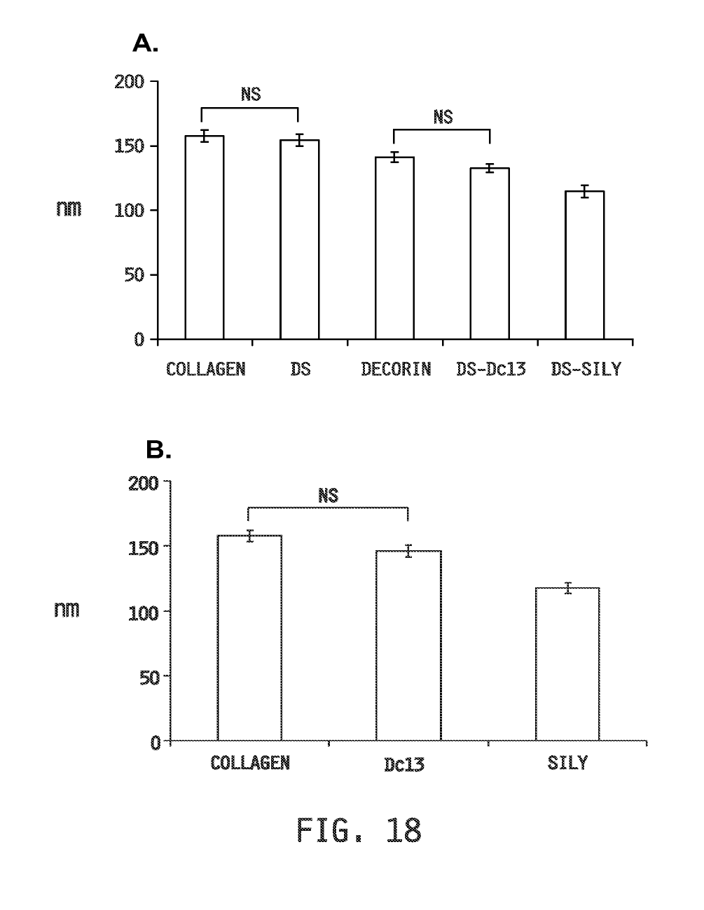

Using a modification of EXAMPLE 18 fibril diameters were measured by 8cryo-SEM. Fibril diameters from cryo-SEM images taken at 20,000× were measured using ImageJ software (NIH). At least 45 fibrils were measured for each treatment. Results are presented as Avg.±S.E. Statistical analysis was performed using DesignExpert software (StatEase) with α=0.05. The results are shown in FIG. 18. Decorin and synthetic peptidoglycans significantly decrease fibril diameter over collagen or collagen+dermatan sulfate. Compared to collagen alone, free peptide Dc13 does not affect fibril diameter while free SILY results in a decrease in fibril diameter.

### Example 25

### Cell Culture and Gel Compaction

Human coronary artery smooth muscle cells (HCA SMC) (Cascade Biologics) were cultured in growth medium (Medium 231 supplemented with smooth muscle growth factor). Cells from passage 3 were used for all experiments. Differentiation medium (Medium 231 supplemented with 1% FBS and 1× pen/strep) was used for all experiments unless otherwise noted. This medium differs from manufacturer protocol in that it does not contain heparin.

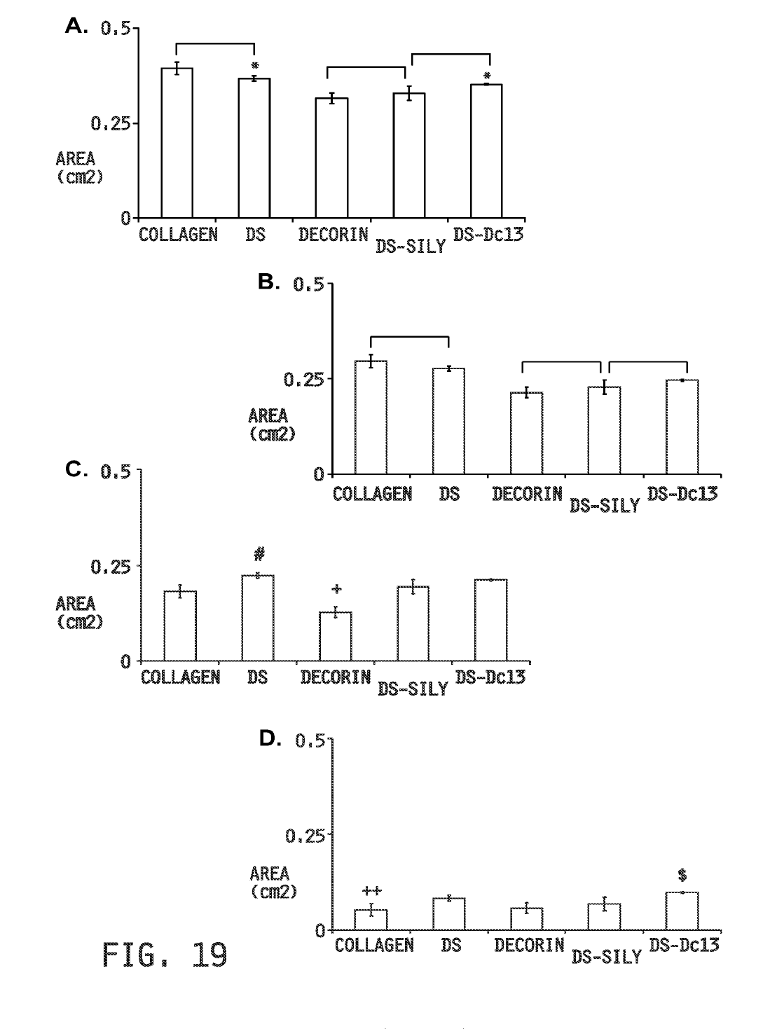

Collagen gels were prepared with each additive as described with the exception that the 1×PBS sample addition was omitted to accommodate the addition of cells in media. After incubating on ice for 30 min, HCA SMCs in differentiation medium were added to the gel solutions to a final concentration of 1×10⁶ cells/mL. Gels were formed in quadruplicate in 48-well non-tissue culture treated plates (Costar) for 6 hrs before adding 500 μL/well differentiation medium. Gels were freed from the well edges after 24 hrs. Medium was changed every 2-3 days and images for compaction were taken at the same time points using a Gel Doc System (Bio-Rad). The cross-sectional area of circular gels correlating to degree of compaction was determined using ImageJ software (NIH). Gels containing no cells were used as a negative control and cells in collagen gels absent additive were used as a positive control. The results are shown in FIG. 19. By day 10 all gels had compacted to approximately 10% of the original gel area, and differences between additives were small. Gels treated with DS-Dc13 were slightly, but significantly, less compact than gels treated with decorin or collagen but compaction was statistically equivalent to that seen with DS and DS-SILY treated gels.

### Example 26

### Measurement of Elastin

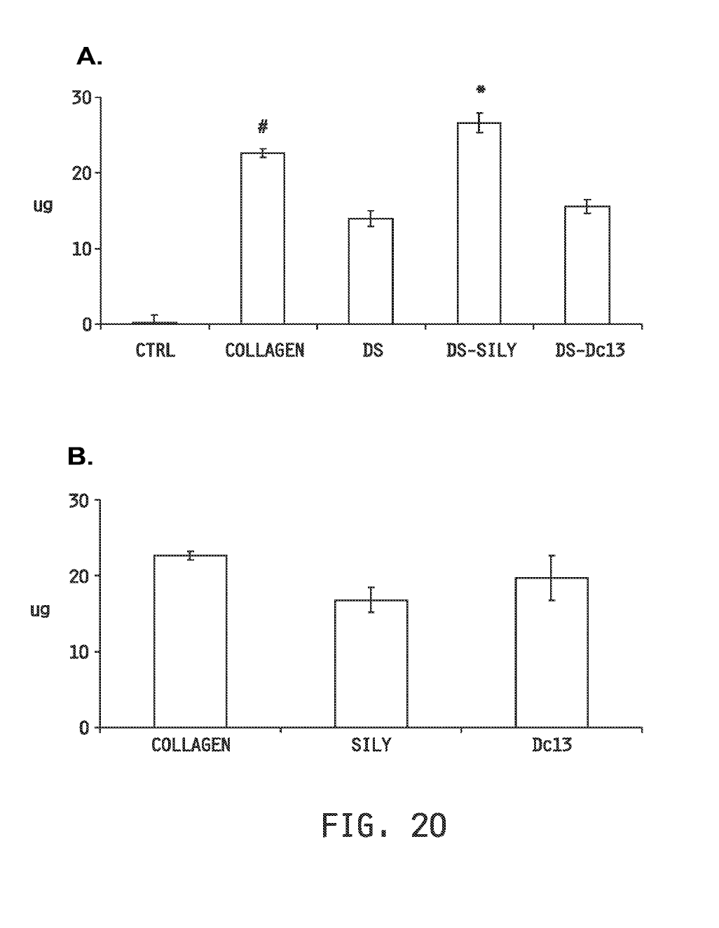

Collagen gels seeded with HCA SMCs were prepared as described in EXAMPLE 25. Differentiation medium was changed every three days and gels were cultured for 10 days. Collagen gels containing no cells were used as a control. Gels were rinsed in 1×PBS overnight to remove serum protein, and gels were tested for elastin content using the Fastin elastin assay per manufacturers protocol (Biocolor, County Atrim, U.K.). Briefly, gels were solubilized in 0.25 M oxalic acid by incubating at 100° C. for 1 hr. Elastin was precipitated and samples were then centrifuged at 11,000×g for 10 min. The solubilized collagen supernatant was removed and the elastin pellet was stained by Fastin Dye Reagent for 90 min at room temperature. Samples were centrifuged at 11,000×g for 10 min and unbound dye in the supernatant was removed. Dye from the elastin pellets was released by the Fastin Dye Dissociation Reagent, and 100 μL samples were transferred to a 96-well plate (Costar). Absorbance was measured at 513 nm, and elastin content was calculated from an α-elastin standard curve. The results of these assays are shown in FIG. 20. Treatment with DS-SILY significantly increased elastin production over all samples. Treatment with DS and DS-Dc13 significantly decreased elastin production over untreated collagen. Control samples of collagen gels with no cells showed no elastin production.

### Example 27

### Effect of Heparin or Heparin-SILY on Platelet Interaction

Collagen was immobilized on glass cover slides (18 mm) by incubating slides with collagen at 2 mg/mL in 10 mM HCl for 1 hr at 37° C. Slides were then washed with 1×PBS and stored at 4° C. in 1×PBS for 24 hrs until further testing. Untreated glass cover slides were used as a negative control. Slides were placed into a 48-well non tissue-culture treated plate (Costar) with the collagen surface facing up. Heparin or Heparin-SILY were dissolved in 1×PBS to a concentration of 100 μM and incubated at 100 μL/well for 30 min at 37° C. Unbound heparin or Heparin-SILY were aspirated and the surfaces were washed with 1 mL 1×PBS. Collagen immobilized slides incubated with 1×PBS containing no additive were used as a positive control.

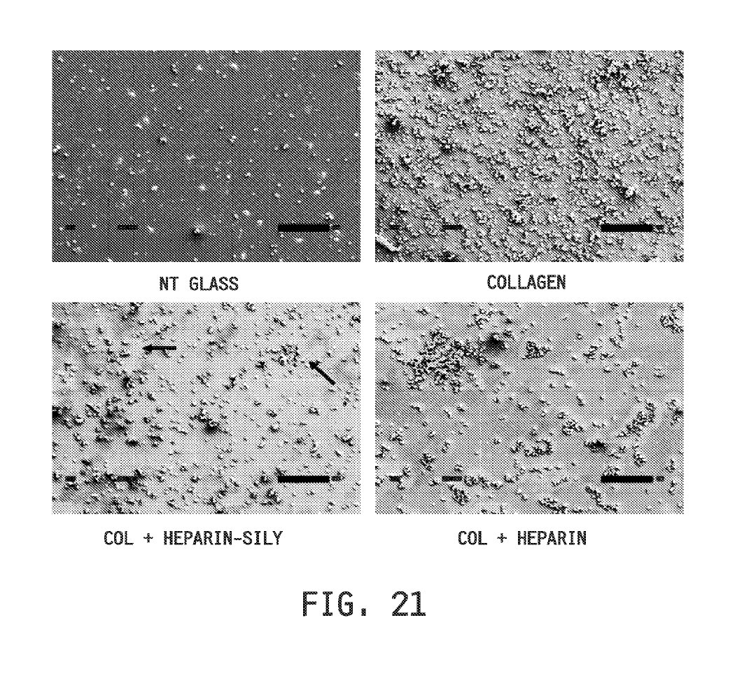

Whole human blood was centrifuged at 800×g for 15 min and 100 μL of platelet-rich plasma was removed from the huffy coat layer and added to each well. After incubating for 1 hr at 37° C., platelet-rich plasma was removed from the wells and the wells were gently washed with 1×PBS to remove unbound cells. Slides were fixed with 5% glutaraldehyde for 1 hr at room temperature, rinsed, and lyophilized before imaging. Slides were gold sputter coated for 3 min and imaged at 200× on a JEOL 840 SEM. The results are shown in FIG. 21. This images show that treatment with the heparin-SILY conjugate affects platelet cell binding to collagen.

### Example 28

### Cryo-SEM Measurement of Fibril Density

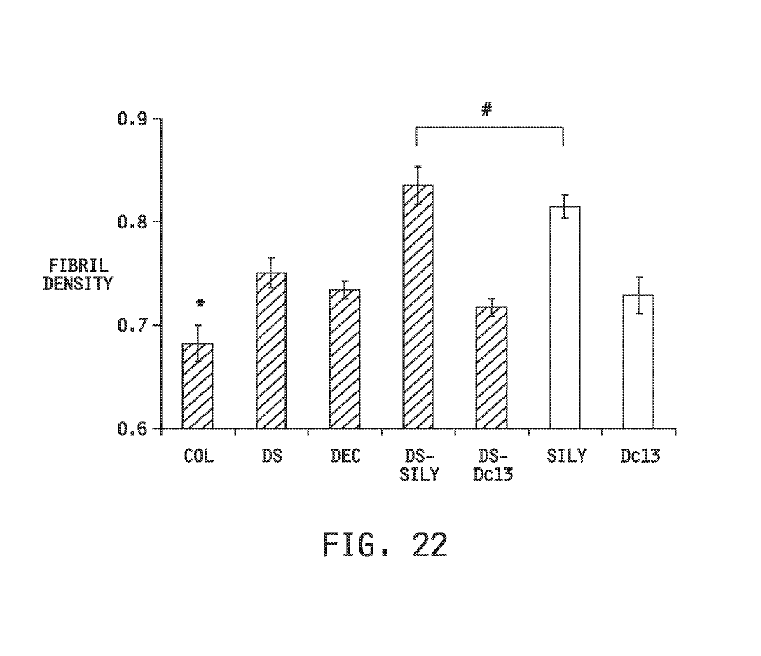

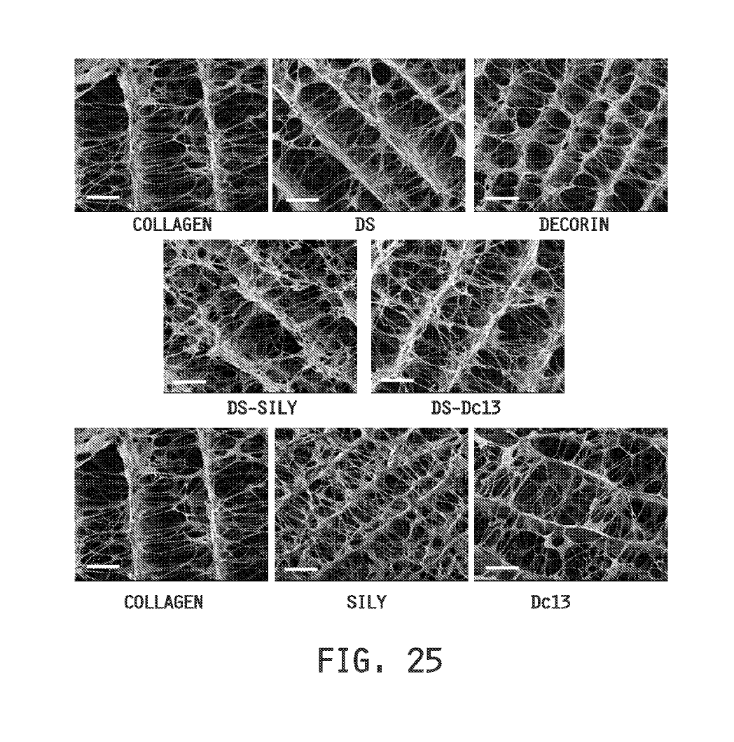

Collagen gels were formed in the presence of each additive at a 10:1 molar ratio, as described in EXAMPLE 15, directly on the SEM stage, processed, and imaged as described. Images at 10,000× were analyzed for fibril density calculations. Images were converted to 8-bit black and white, and threshold values for each image were determined using ImageJ software (NIH). The threshold was defined as the value where all visible fibrils are white, and all void space is black. The ratio of white to black area was calculated using MatLab software. All measurements were taken in triplicate and thresholds were determined by an observer blinded to the treatment. Images of the gels are shown in FIG. 25 and the measured densities are shown in FIG. 22.

### Example 29

### Viscoelastic Characterization of Gels Containing Dc13 or DS-Dc13

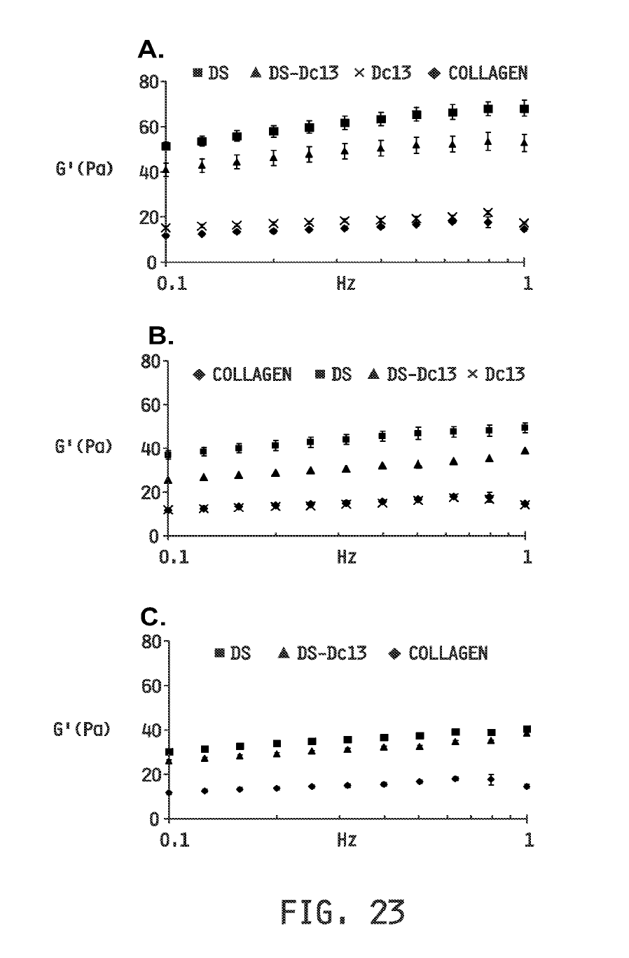

Collagen gels were prepared, as in EXAMPLE 15. Viscoelastic characterization was performed as described in EXAMPLE 16 on gels formed with varying ratios of collagen to additive (treatment). Treatment with dermatan sulfate or dermatan-Dc13 conjugate increase the stiffness of the resulting collagen gel over untreated collagen as shown in FIG. 23.

### Example 30

### Cell Proliferation and Cytotoxicity Assay

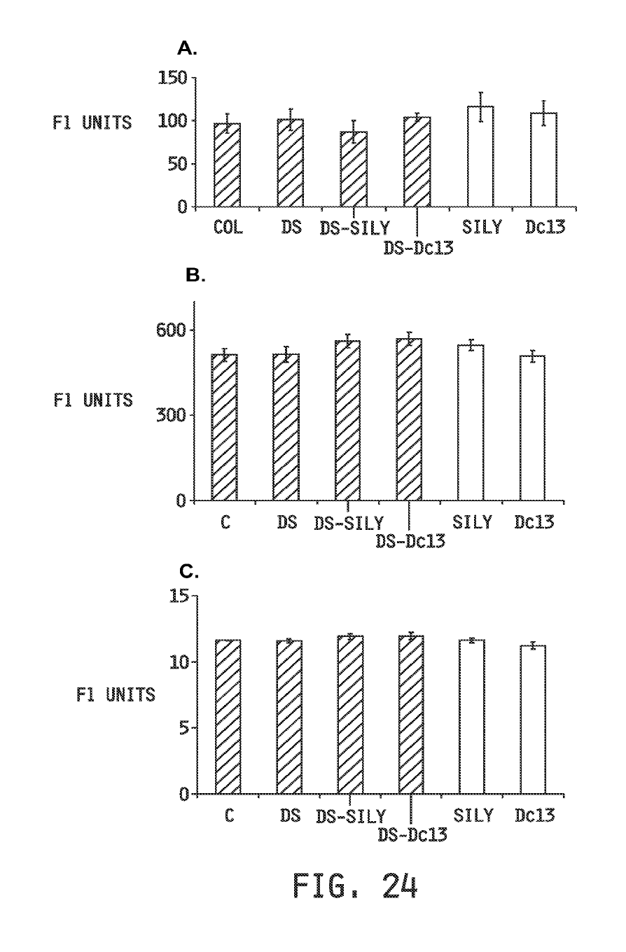

HCA SMCs, prepared as in EXAMPLE 25, were seeded at 4.8×10⁴ cells/mL in growth medium onto a 96-well tissue-culture black/clear bottom plate (Costar) and allowed to adhere for 4 hrs. Growth medium was aspirated and 600 μL of differentiation medium containing each additive at a concentration equivalent to the concentration within collagen gels (1.4×10⁻⁶ M) was added to each well. Cells were incubated for 48 hrs and were then tested for cytotoxicity and proliferation using Live-Dead and CyQuant (Invitrogen) assays, respectively, according to the manufacturer's protocol. Cells in differentiation medium containing no additive were used as control. The results are shown in FIG. 24 indicating that none of the treatments demonstrated significant cytotoxic effects.

### Example 31

### Inhibition of Platelet Binding and Platelet Activation to Collagen Type I

### Microplate Preparation

Type I fibrillar collagen (Chronolog, Havertown, Pa.) was diluted in isotonic glucose to a concentration of 20-100 μg/mL. 50 μL of collagen solution was added to each well of a high bind 96-well plate. The plate was incubated overnight at 4° C., and then rinsed 3× with 1×PBS.

Peptidoglycan was diluted in 1×PBS at concentrations of 25 μM to 50 μM and 50 μL solution was added to the collagen coated wells. Controls of GAG, peptide, or PBS were also added to collagen coated wells as controls. Treatments were incubated at 37° C. with shaking at 200 rpm for 30 min. Wells were then rinsed 3× with 1×PBS, including a 20 min rinse with 200 rpm shaking to remove unbound treatment molecule.

### Platelet Preparation and Activation

Human whole blood was collected from healthy volunteers by venipuncture following the approved Purdue IRB protocol and with informed consent. The first 5 mL of blood was discarded as it can be contaminated with collagen and other proteins, and approximately 15 mL was then collected into citrated glass vacutainers (BD Bioscience). Blood was centrifuged in the glass tube for 20 min at 200×g at 20° C. The top layer of the centrifuged blood, the platelet rich plasma (PRP), was used for platelet experiments. PRP (50 μL/well) was added to the microplate and allowed to incubate for 1 hr at room temperature without shaking.

After 1 hour of incubation, the PRP was removed from each well and added to a microcentrifuge tube containing 5 μL ETP (107 mM EDTA, 12 mM theophylline, and 2.8 μM prostaglandin E1) to inhibit further platelet activation. These tubes were spun at 4° C. for 30 min at 1900×g to pellet the platelets. The supernatant (platelet serum) was collected for ELISA studies to test for the presence of platelet activation markers PF-4 and Nap-2.

### Platelet Adherence

After the PRP was removed from the wells of the collagen/treatment coated plates, the wells were rinsed 3× with 0.9% NaCl for 5 min each shaking at 200 rpm. Platelet adherence was quantified colormetrically or visualized fluorescently.

### Colormetric Assay

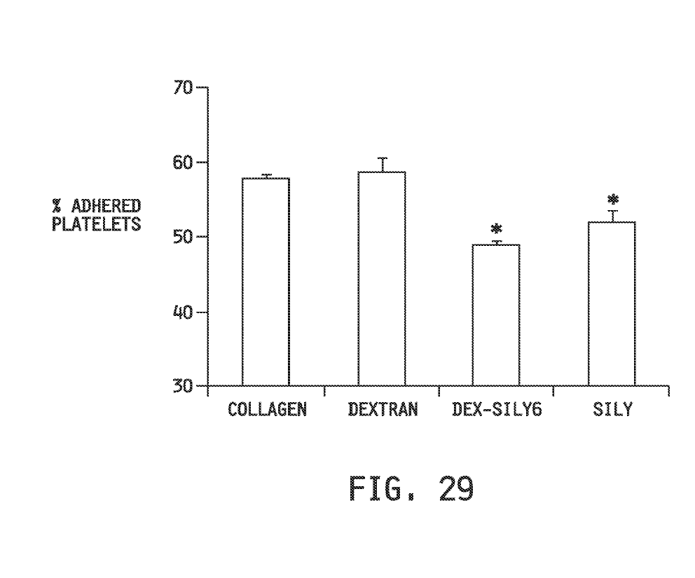

140 μL of a sodium citrate/citric acid buffer (0.1 M, pH 5.4) containing 0.1% Triton X-100 and 1 mg/mL p-nitrophenyl phosphate was added to each well. The background absorbance was measured at 405 nm. The plate was then incubated for 40 min at room temperature with shaking at 200 rpm. The Triton X-100 creates pores in the cells, allowing p-nitrophenyl phosphate to interact with acid phosphatase in the platelets to produce p-nitrophenol. After 40 min of incubation, 100 μL of 2 M NaOH was added to each well. The pH change stops the reaction by inactivating acid phosphatase, and also transforms the p-nitrophenol to an optically active compound. The absorbance was then read at 405 nm and correlated to the number of adhered platelets. The results are shown in FIG. 29.

### Fluorescent Assay

Adhered platelets were fixed by incubation with 4% paraformaldehyde for 10 min at room temperature. The platelets were permeabilized with 0.1% Triton X-100 for 5 min. Platelet actin was labeled by incubation with phalloidin-AlexaFluor 488 (Invitrogen) containing 1% BSA for 30 min. The wells were rinsed 3× with 1×PBS, and the adhered platelets were imaged using an upright fluorescent microscope using a DAPI filter.

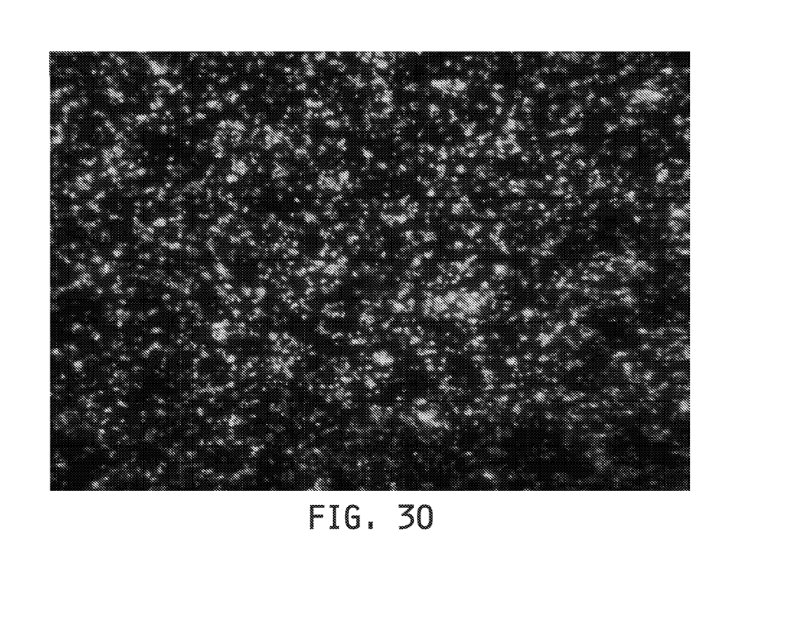

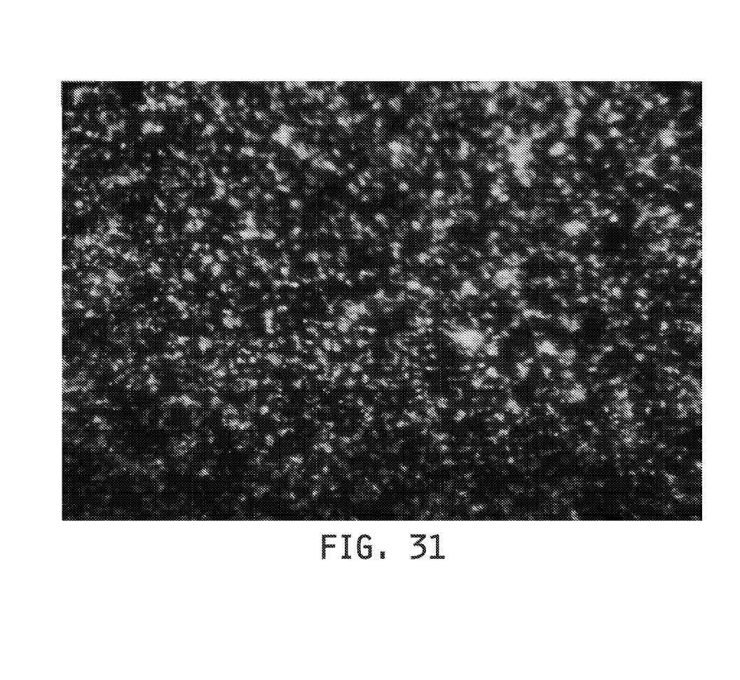

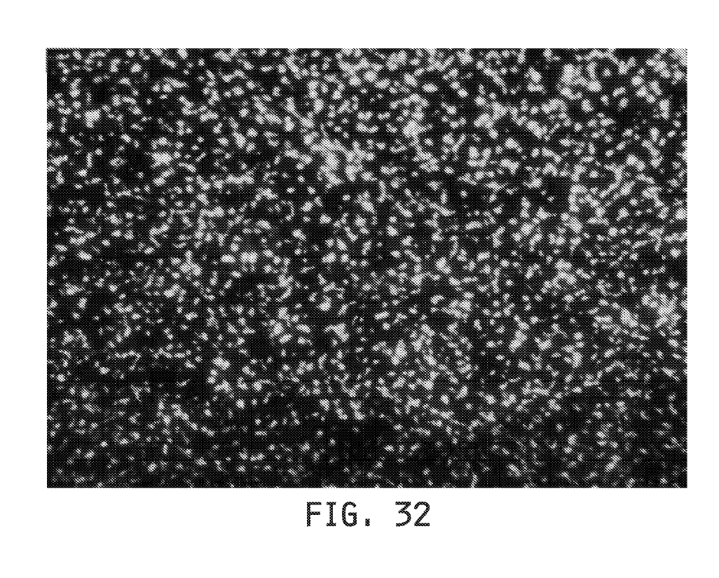

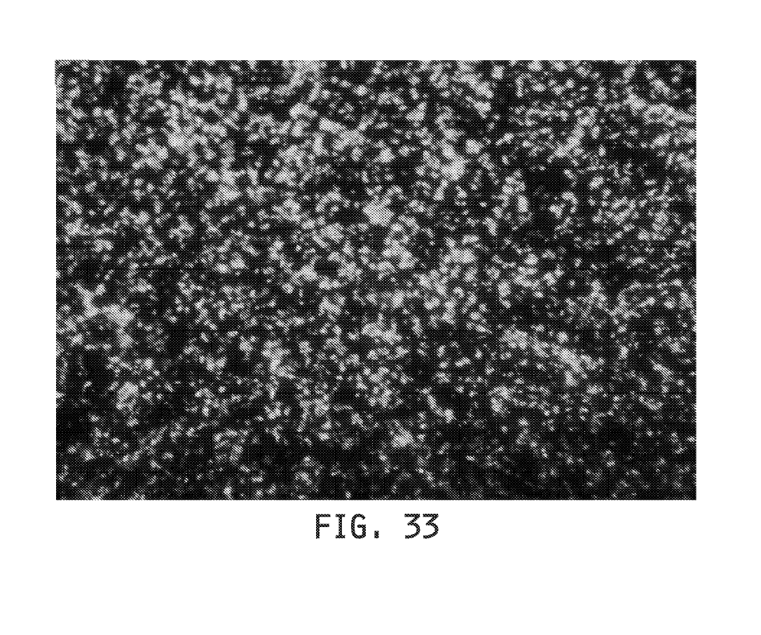

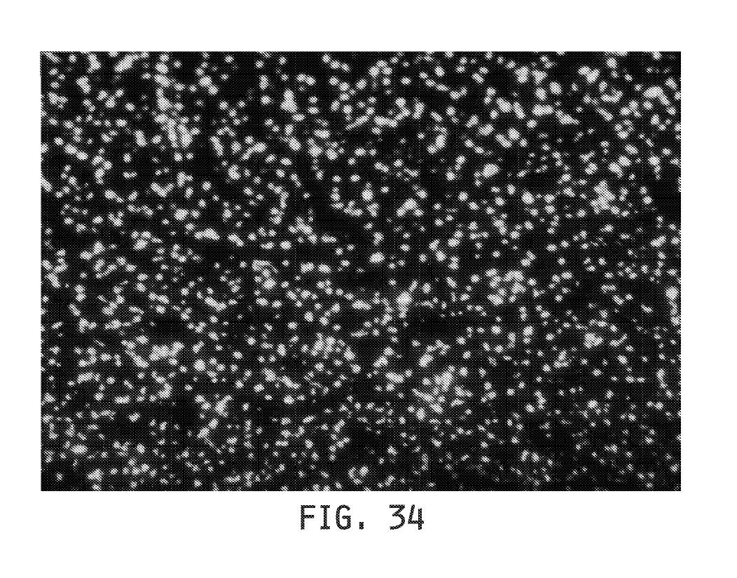

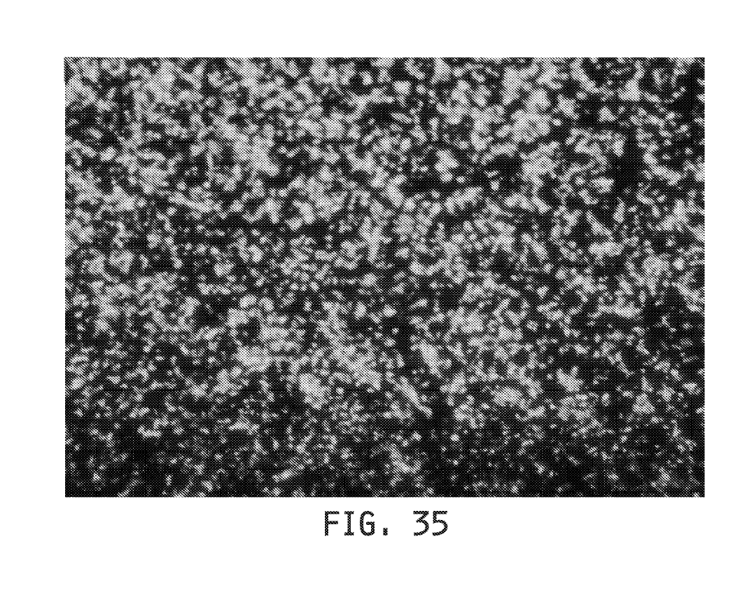

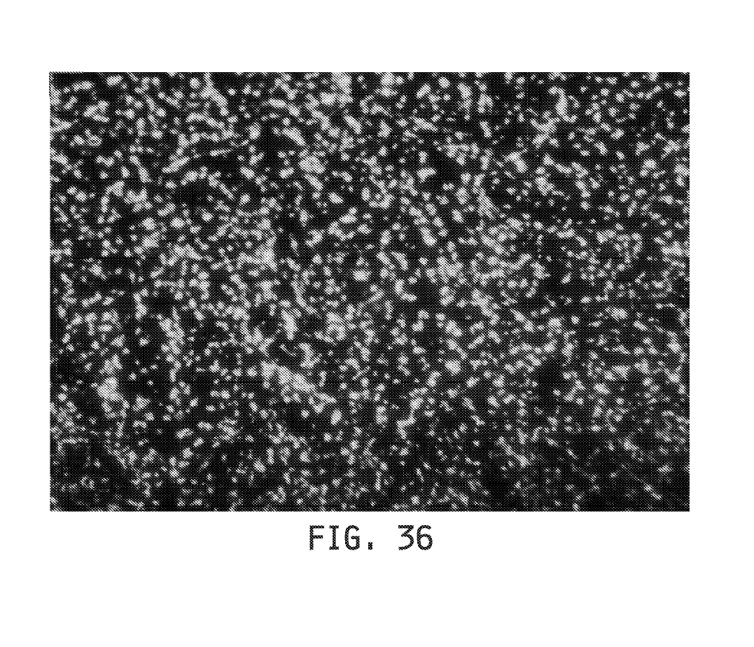

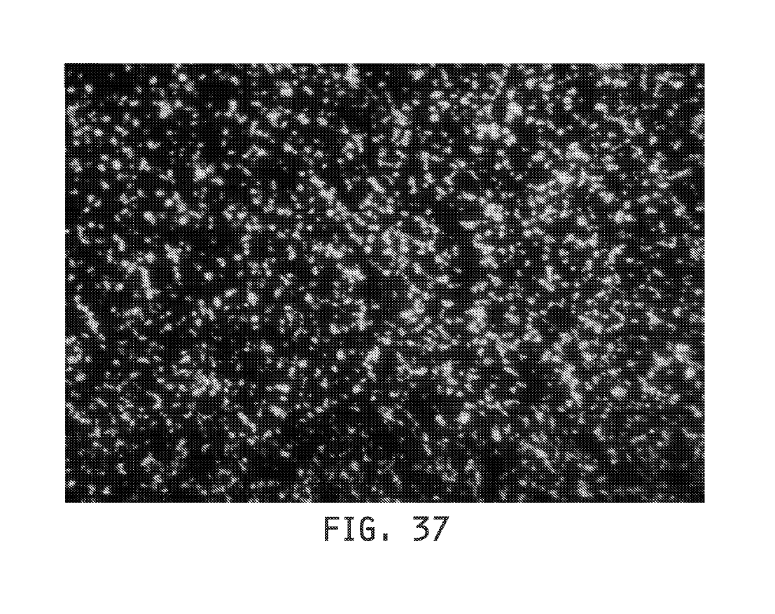

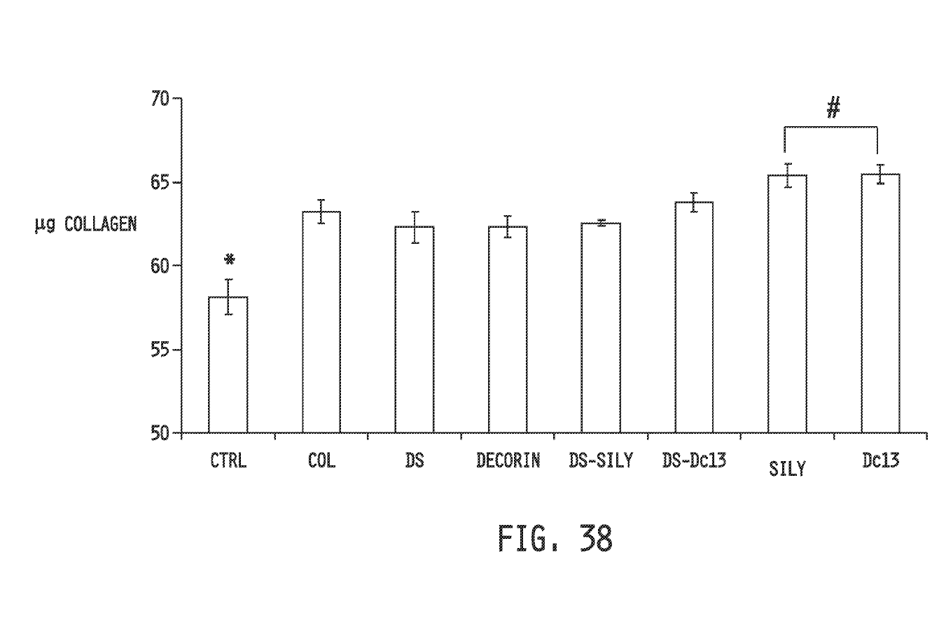

See FIGS. 30 to 39 for results. Platelet aggregation on untreated collagen surfaces is indicated by blurred images resulting from clumped platelets. Without being bound by theory, it is believed that clumping of platelets in the z-direction (perpendicular to the plate surface) prevents image capture in one focal plane. On treated surfaces, reduced platelet aggregation results in less clumping (fewer platelets in the z-direction), and focused images can be captured at the plate surface. These images show that treatment with the synthetic peptidoglycans reduces adhesion of platelet cells to collagen,

### Detection of Platelet Activation Markers

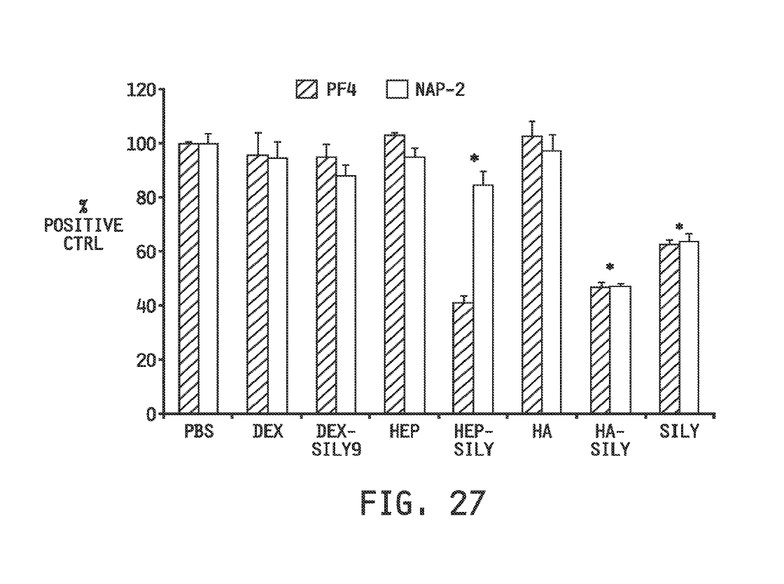

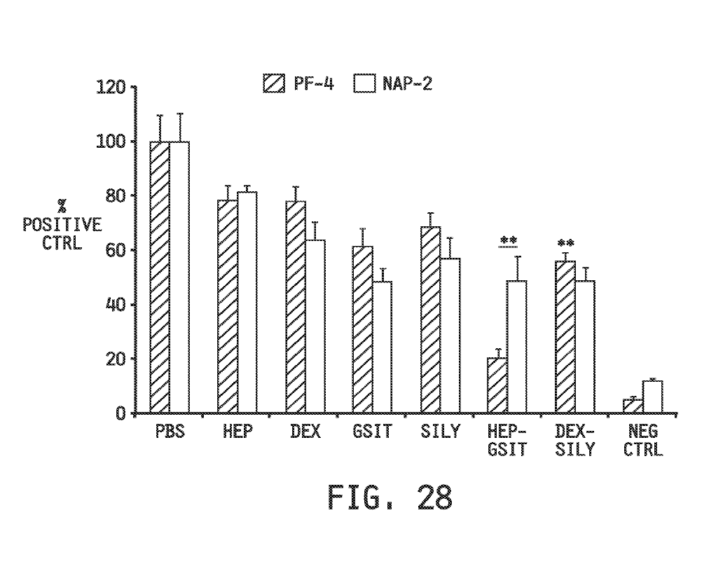

The supernatant (platelet serum) obtained after pelleting the platelets was used to determine released activation factors. Platelet factor 4 (PF-4) and β-thromboglobulin (Nap-2) are two proteins contained within alpha granules of platelets which are released upon platelet activation. Sandwich ELISAs were utilized in order to detect each protein. The components for both sandwich ELISAs were purchased from (R&D Systems) and the provided protocols were followed. The platelet serum samples were diluted 1:10,000-1:40,000 in 1% BSA in 1×PBS so the values fell within a linear range. The results shown in FIGS. 27 and 28 show that treatment with synthetic peptidoglycans decreases platelet activation by collagen type I.

### Example 32

### Inhibition of Platelet Binding and Platelet Activation to Collagen Type III and Type I

The method according to EXAMPLE 31 was used with the following modification.

### Microplate Preparation

Type I collagen (rat tail collagen, BD Biosciences) and type III collagen (Millipore) were combined on ice with NaOH, 1×PBS, and 10×PBS to physiological conditions. The total collagen concentration was 1 mg/mL with 70% type I collagen and 30% type III collagen. 30 μL of the collagen solution was pipetted into each well of a 96-well plate. The plate was incubated at 37° C. in a humidified incubator for one hour, allowing a gel composed of fibrillar collagen to form in the wells. The wells were rinsed 3× with 1×PBS.

Peptidoglycan was diluted in 1×PBS at concentrations of 25 vμM and 50 vμL solution was added to the collagen coated wells. Controls of GAG, peptide, or PBS were also added to collagen coated wells as controls. Combinations of peptidoglycan or peptide were composed of 25 μM of each molecule in 1×PBS. Treatments were incubated at 37° C. with shaking at 200 rpm for 30 min. Wells were then rinsed 3× with 1×PBS, including a 10 min rinse with 200 rpm shaking to remove unbound treatment molecule.

The results of the platelet activation inhibition measurements shown in FIG. 39 demonstrate that the synthetic peptidoglycans inhibit platelet cell activation by a mixture of collagen Type I and Type III.

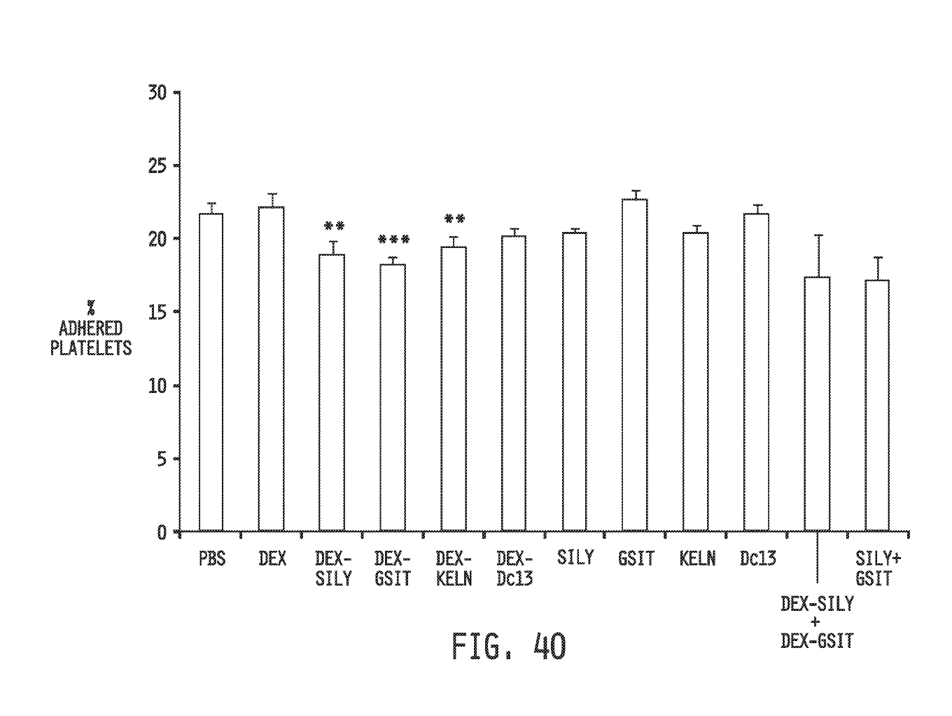

The results shown in FIG. 40 demonstrate that the peptidoglycans inhibit platelet cell binding to collagen Type I and Type III mixtures.

### Example 33

### Peptidoglycan Synthesis

The peptides used to synthesize the peptidoglycans described in this Example and the following Examples were synthesized by GenScript (Piscataway, N.J.). The peptidoglycan was synthesized as described with modifications. Dermatan sulfate (DS) was oxidized by periodate oxidation in which the degree of oxidation was controlled by varying amounts of sodium meta-periodate. After oxidizing at room temperature for 2 hours protected from light, the oxidized DS was desalted into 1×PBS pH 7.2 by size exclusion chromatography using a column packed with Bio-gel P-6 (BioRad). The heterobifunctional crosslinker BMPH (Thermo Fischer Scientific) was added to oxidized DS in 30 fold molar excess to DS, and was reacted for 2 hours at room temperature protected from light. The intermediate product DS-BMPH was then purified of excess BMPH by size exclusion as described with 1×PBS pH 7.2 as running buffer. The number of BMPH crosslinkers attached to DS was calculated by the consumption of BMPH determined from the 215 nm peak area of the excess BMPH peak. A standard curve of BMPH was generated to calculate excess BMPH. The free peptide SILY was dissolved into water at a concentration of 2 mg/mL and was added in 1 molar excess to the number of attached BMPHs and was reacted for 2 hours at room temperature. The final product DS-SILYₙ was purified by size exclusion using a column packed with Sephadex G-25 medium (GE Lifesciences) with Millipore water as the running buffer. The final product was immediately frozen, lyophilized, and stored at −20° C. until further testing.

A biotin labeled version of the peptidoglycan was synthesized by reacting 2 moles of SILY_{biotin} per mole of DS-BMPH for 1 hour, followed by addition of unlabeled SILY to a 1 molar excess per attached BMPH. After unlabeled SILY was added, the reaction continued for 2 hours at room temperature before purification. Biotin labeled peptidoglycan is designated as DS-SILY_{n-biotin} where n is the total number of SILY peptides per molecule. For DS-SILY_{4-biotin} only biotin labeled SILY was reacted, rather than unlabeled biotin.

### Example 34

### Purification and Characterization of DS-BMPH

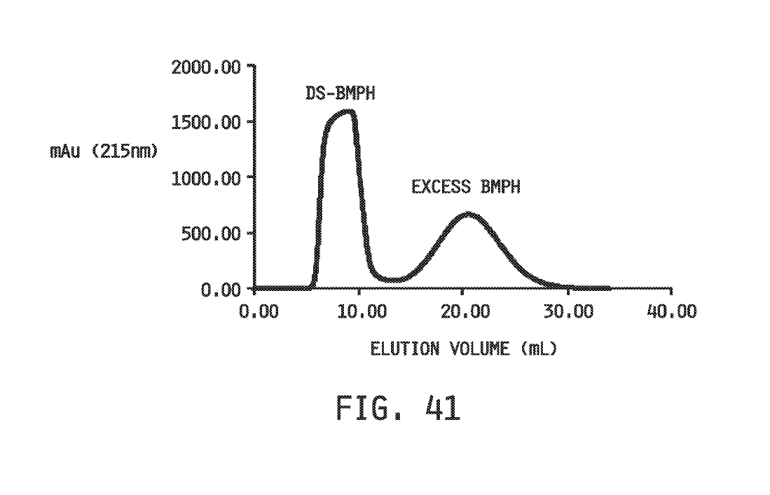

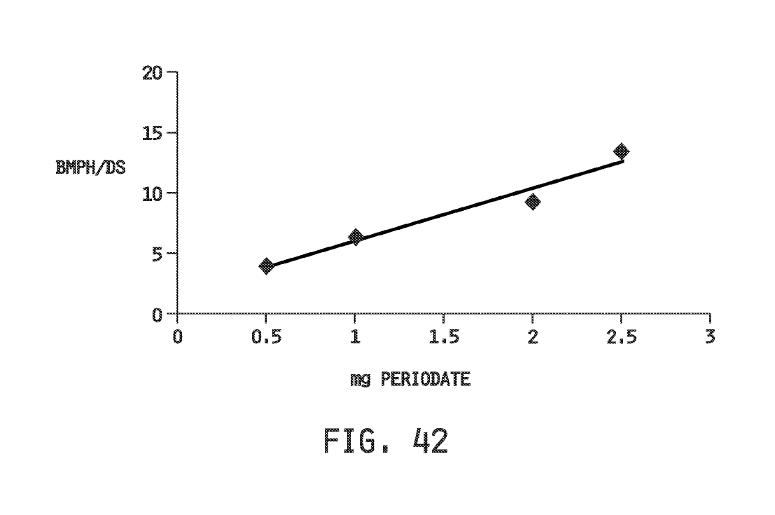

Oxidized DS was coupled to BMPH as described and purified of excess BMPH by size exclusion chromatography. As shown in FIG. 41, the amount of excess BMPH is calculated by integrating the excess BMPH peak and comparing to a standard curve for BMPH. As shown in FIG. 42, by varying the amount of sodium meta-periodate, the number of BMPH crosslinkers per DS chain increases linearly.

### Example 35

### Binding Affinity of Peptidoglycan to Collagen

Fibrillar collagen (Chronolog, Havertown, Pa.) was coated onto the surface of a 96-well high bind plate (Costar) at a concentration of 50 μg/mL diluted in isotonic glucose. Plates were covered and incubated overnight at 4° C. Unbound collagen was removed by rinsing 3 times with 1×PBS pH 7.4. Plates were then blocked with 1% BSA for 3 hours at room temperature. Peptidoglycan was dissolved at varying concentrations in 1×PBS pH 7.4 containing 1% BSA and were immediately added to the collagen surfaces, and allowed to incubate for 15 min at room temperature. Plates were then rinsed 3 times with 1×PBS pH 7.4 containing 1% BSA. Streptavidin-HRP solution (R&D Systems, Minneapolis, Minn.) was then added to the plates and incubated for 20 minutes at room temperature. Unbound streptavidin was rinsed 3 times with 1×PBS pH 7.4 and 100 μL/well of color evolving solution (stabilized hydrogen peroxide and stabilized tetramethylbenzidine, R&D Systems, Minneapolis, Minn.) was added to each well and incubated for 20 minutes at room temperature protected from light. The color evolving reaction was stopped with 50 μL 2N sulfuric acid and absorbance was measured at 450 nm using an M5 UV Vis Spectrophotometer (Molecular Devices). Plate imperfections (540 nm) were subtracted from absorbance values.

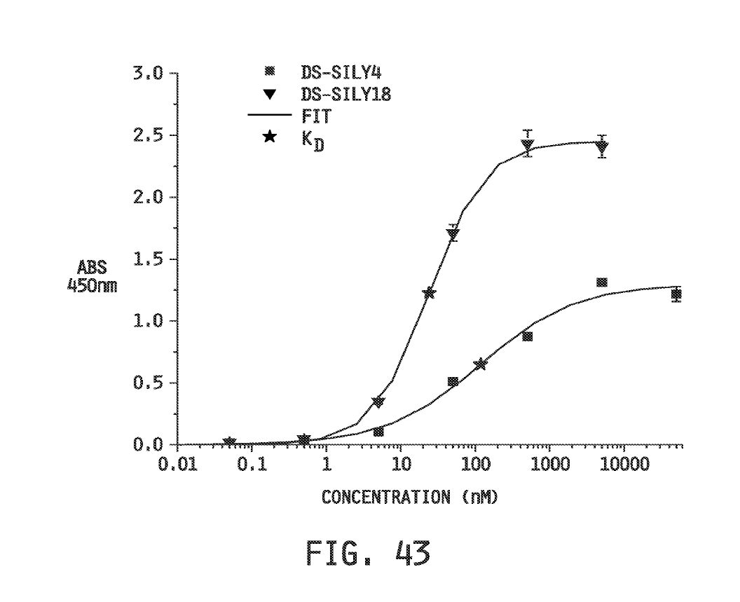

The binding affinities of biotin labeled peptidoglycans, labeled using protocols known in the art, DS-SILY₄ and DS-SILY₁₈ were calculated by fitting the saturation binding curves and calculating the inflection point. As shown in FIG. 43, DS-SILY₄ and DS-SILY₁₈ bind to fibril collagen with a K_{D} of 118 nM and 24 nM, respectively. By increasing the number of peptides per DS backbone, it is also apparent that more molecules are able to bind to the collagen surface, which is noted by the increased absorbance of DS-SILY₁₈ which does not contain more biotin label than DS-SILY₄. Consequently it is expected that DS-SILY₁₈ will show improved platelet inhibition since it can form a denser covering of the collagen surface.

### Example 36

### Inhibition of Platelet Binding and Activation

Type I fibrillar collagen from Chronolog was diluted in isotonic glucose to a concentration of 50 μg/mL. 50 μL of collagen solution was added to each well of a high bind 96-well plate. The plate was incubated overnight at 4° C., and then rinsed 3× with 1×PBS. For microplate assays, peptidoglycan was diluted in 1×PBS at concentrations between 0.0001 μM to 100 μM and 50 μL solution was added to the collagen coated wells. DS, peptide, or 1×PBS were also added to collagen coated wells as controls. Treatments were incubated at 37° C. with shaking at 200 rpm for 15 min. Wells were then rinsed of unbound treatment by removing the treatment solution, adding PBS, and shaking the wells for 24 hours. During the 24 hours, PBS solution was changed 3 times.

Human whole blood was collected from healthy volunteers by venipuncture. The first 5 mL of blood was discarded and approximately 20 mL was then collected into citrated glass vacutainers (BD Bioscience). Blood was centrifuged in the glass tube for 20 min at 200 g at 25° C. The top layer of the centrifuged blood, the platelet rich plasma (PRP), was used for platelet experiments.

PRP (50 μL/well) was added to the microplate for 1 hour at room temperature without shaking. After 1 hour of incubation, 45 μL of PRP was removed from each well and added to a microcentrifuge tube containing 5 mL ETP (107 mM EDTA, 12 mM theophylline, and 2.8 mM prostaglandin E₁) to inhibit further platelet activation. These tubes were spun at 4° C. for 30 min at 2000 g to pellet the platelets. The supernatant (platelet serum) was collected for ELISA studies to test for the presence of platelet activation markers PF-4 and NAP2. Sandwich ELISAs were utilized in order to detect each protein. The components for both sandwich ELISAs were purchased from R&D Systems and the provided protocols were followed. Platelet serum was diluted 10,000 times in 1% BSA in 1×PBS for values to fall within a linear range.

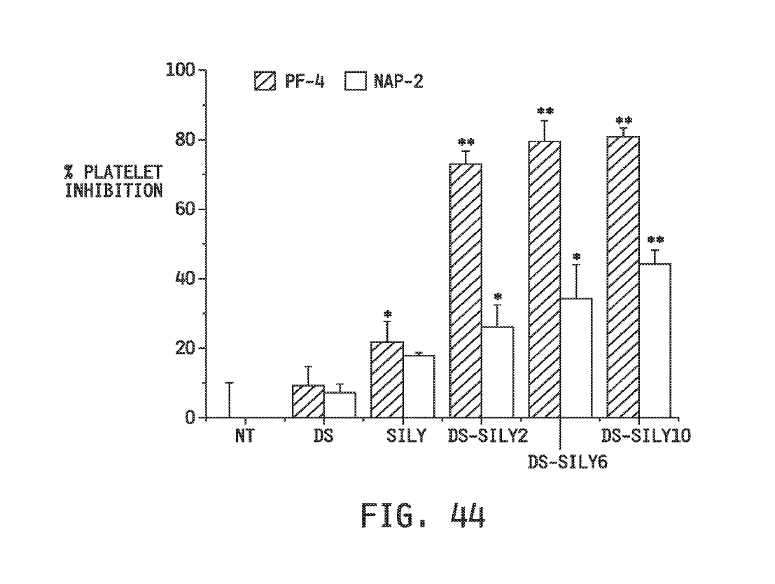

Platelet activation was measured through release of platelet factor 4 (PF4) and β-thromboglobulin (NAP2). FIG. 44 shows the % decrease in platelet activation by different treatments. At concentrations as high as 50 μM, DS and SILY had little to no effect on inhibiting platelet activation. Unlike individual DS or SILY, DS-SILY was able to inhibit collagen mediated platelet activation. As the number of SILY peptides per DS molecule increased from 2 to 10, the inhibition of platelet activation also increased. Since the high SILY/DS ratio is expected to provide higher binding affinity to the collagen surface due to multiple interactions, peptidoglycans with the higher SILY/DS ratios were prepared.

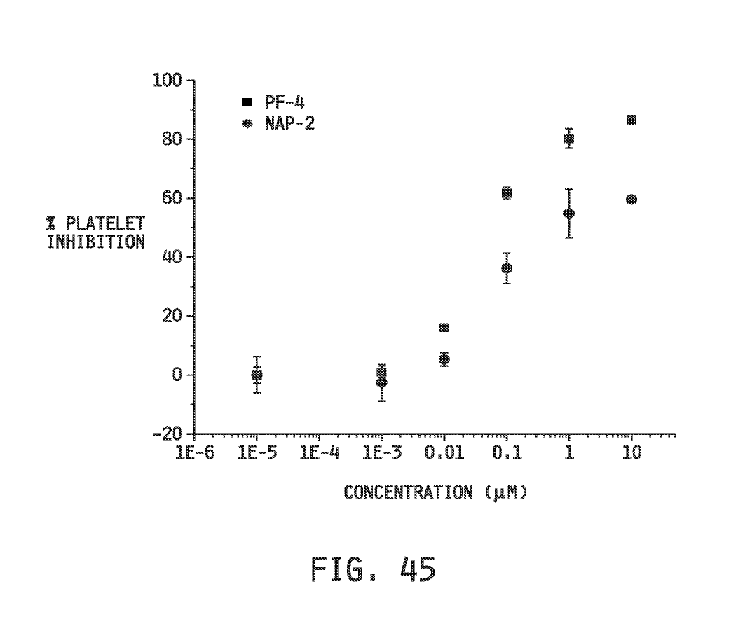

The number of peptides per DS molecule was further increased to 18 to create the peptidoglycan DS-SILY₁₈, and the concentration of molecule needed to inhibit platelet activation was tested. FIG. 45 shows the extent of inhibition of platelet activation by DS-SILY₁₈. The data together suggest that increasing the number of peptides per DS chain further inhibits platelet binding to collagen and platelet activation. Within solubility limits, the number of peptides can be increased to achieve maximal platelet inhibition as well as to reduce diffusion over time, where a higher level of peptidoglycans on the denuded vessel wall is sustained.

### Example 37

### Peptidoglycan Diffusion from Collagen Surface

The peptidoglycan OS-SILY_{18-biotin} was dissolved at 10 μM in 1×PBS pH 7.4 with 2% BSA and was incubated on fibrillar collagen coated plates prepared as described. The plate was incubated at 37° C. on an orbital shaker and was rinsed extensively with 1×PBS pH 7.4. At various time points up to 11 days, OS-SILY_{18-biotin} was detected on the surface as described for the binding affinity studies. The curve of diffusion of peptidoglycan from the collagen surface was fitted using a hyperbolic decay.

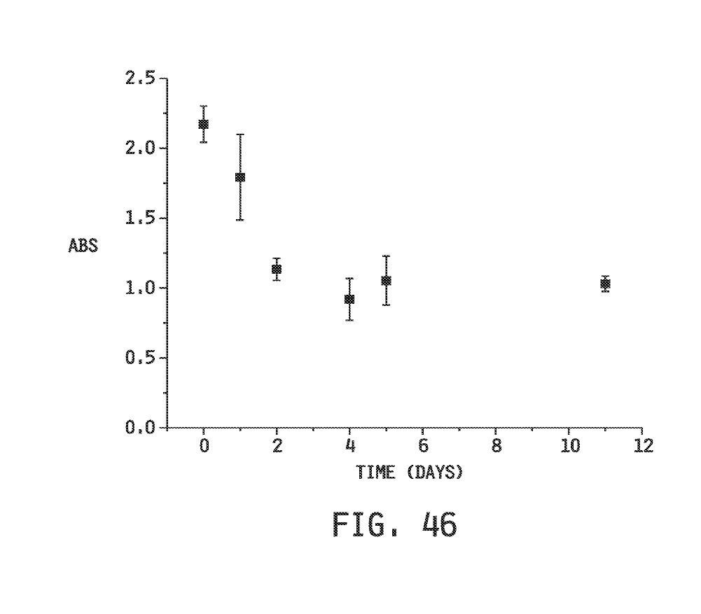

The diffusion of the peptidoglycan DS-SILY₁₈ from a collagen surface was measured by incubating at 37° C. and rinsing extensively in order to mimic blood flow in vivo. Peptidoglycan was incubated on fibrillar collagen surfaces and detected by the same methods for calculating binding affinities. As shown in FIG. 46, the peptidoglycan does diffuse to some degree from the collagen surface; however, after 1 week of extensive rinsing, the equivalent of approximately 10 nM remained bound on the surface. It is estimated that complete endothelial cell regrowth occurs within 1 week of balloon injury, and thus this time frame is useful for preventing platelet binding until endothelial cells grow back and provide a permanent cover to the underlying collagen.

### Example 38

### Endothelial Cell Proliferation

Endothelial regrowth is essential for restoring the healthy vessel and providing a permanent barrier covering the underlying collagen. Currently available drug-eluting stents for example, prevent endothelial regrowth and have consequently shown a new set of complications such as late-stent thrombosis. The peptidoglycan was tested at varying concentrations to determine if it inhibited regrowth of the endothelium. The peptidoglycan was physically bound to collagen through peptide-collagen interactions, susceptible to removal by competition binding, and thus was replaced by endothelial cells growing back over the collagen layer.

Human coronary artery endothelial cells (ECs) (Lonza, Walkersville, Md.) were seeded in 96-well plates at a cell density of 1.5×10³ cells/well. Cells were allowed to adhere to the surface for 24 hours and adherent ECs were stained using cell tracker green (Invitrogen, Carlsbad, Calif.). Initial cell number was determined by measuring fluorescence of each well with 492 nm excitation and 517 nm emission.

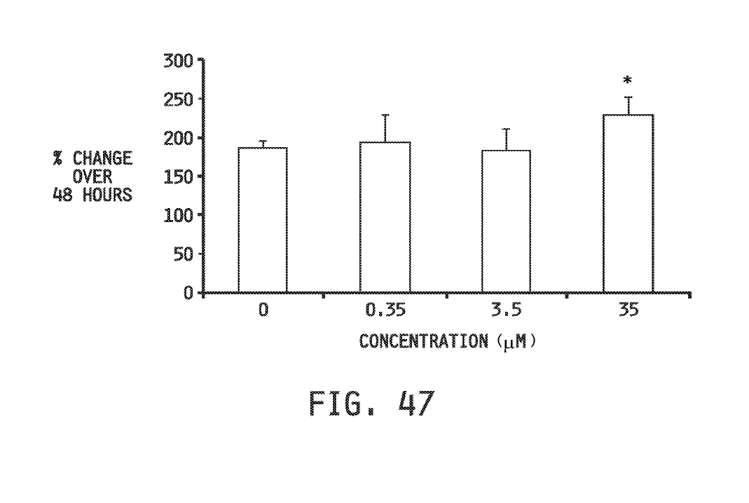

DS-SILY₁₈ was solubilized in water at a concentration of 175 μM, and diluted in water 10 fold for concentrations of 175, 17.5, and 1.75 μM. The DS-SILY solution was diluted 5 fold with cell media for final concentrations of 35, 3.5, and 0.35 mM. The control consisted of diluting water 5 fold with cell media. 100 μL of media with DS-SILY was added to each well and the cell number was determined 48 hours later by measuring fluorescence. The percent change in cell number in each well was calculated. As shown in FIG. 47, at the highest concentration tested, there was a significant increase in cell proliferation, which suggests that the peptidoglycan promotes endothelial regrowth rather than inhibiting regrowth.

### Example 39

### Endothelial Cell Migration

To mimic the true environment where an area of endothelial cells grown on collagen is denuded, a second test of endothelial cell migration was performed. Fibrillar collagen (Chronolog, Havertown, Pa.) was coated in wells of 96-well Oris Cell Migration Kit (Platypus Technologies, Madison, Wis.). Stoppers were inserted into the plate to block an inner circular portion of the cell. Human coronary artery endothelial cells (ECs) (Lonza, Walkersville, Md.) were seeded at 5×10³ cells/well and grown to confluence in the outer portion of the well. Once confluent in the outer portion of the well, the cells were stained with cell tracker green (Invitrogen, Carlsbad, Calif.). The stoppers of the wells were removed, and DS-SILY₁₈ solubilized in 1×PBS was incubated on the exposed collagen surface in the inner portion of the well for 15 min at 37° C. Unbound DS-SILY was rinsed from the surface and cell media was returned to the wells. ECs were allowed to migrate from the outer portion of the wells to the inner portion for 48 hours. Fluorescence measurements of the center of each well were measured using a mask provided with the migration kit so that only the treated inner circular portion of the well was measured.

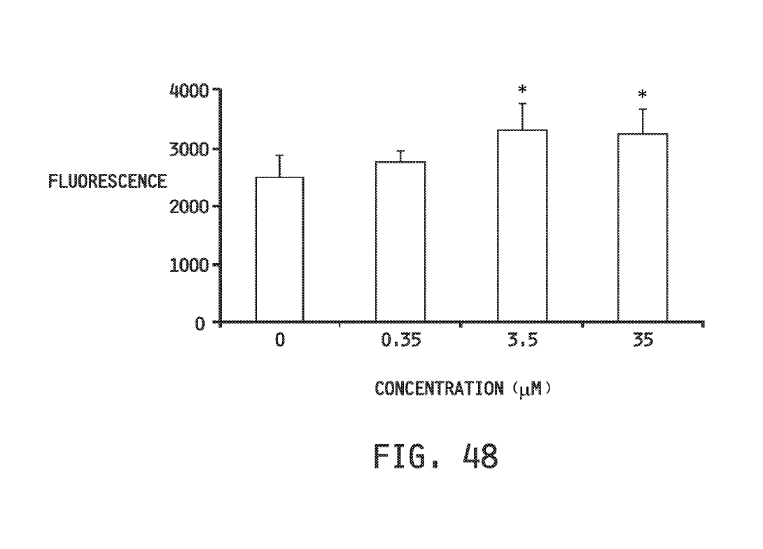

As shown in FIG. 48, the same trend of increased cell number at increasing peptidoglycan concentrations was observed. Together with cell proliferation trends, the data shows that endothelial regrowth is not inhibited by peptidoglycan treatment, but that the peptidoglycan promotes regrowth.

### Example 40

### Platelet Binding to Collagen Under Flow

To mimic physiologic conditions with flowing blood, platelet binding to collagen surfaces under flow conditions was evaluated. Flow kits were obtained from Ibidi (Munchen, Germany). Each channel was coated with fibrillar collagen (Chronolog). Excess collagen was removed from the flow channel by pushing 1×PBS through the channel with a syringe. DS-SILY₁₈ was incubated in the channel at a concentration of 50 μM for 15 min at 37° C., and unbound peptidoglycan was rinsed by pushing 1×PBS through the channel with a syringe. The control channel consisted of collagen not treated with peptidoglycan.

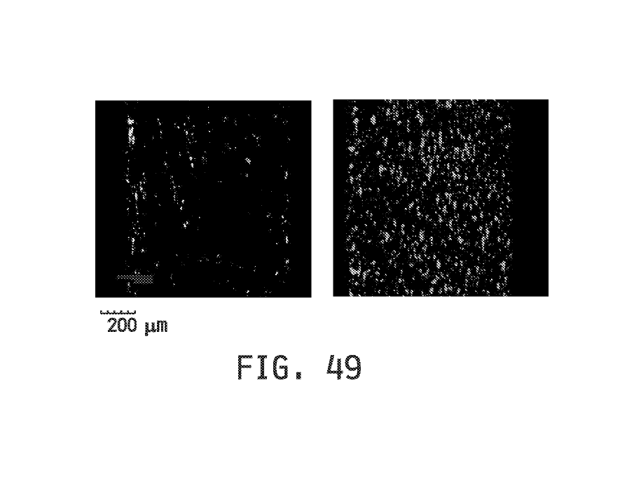

Platelet rich plasma was pushed through the channels at 2 mL/hr for 1 hour, corresponding to a shear stress of 3.55 dynes/cm² and a shear rate of 355 s⁻¹. Unbound platelets were rinsed from the channel by pushing through 1×PBS. Adhered platelets were fixed in the channel with 10% formaldehyde. Platelets were permeabilized and actin filaments were stained with phalloidin-Alexa Fluor 488 (Invitrogen). Representative images of adherent platelets are shown in FIG. 49, which demonstrate that significantly fewer platelets bound to the peptidoglycan treated surface in comparison to untreated collagen.

### Example 41

### In Vivo Ossabaw Pig Studies

In vivo studies were performed on Ossabaw pigs in order to determine the optimal delivery method and concentration of the peptidoglycan as well as to determine preliminary efficacy of the peptidoglycan treatment. Healthy adult Ossabaw pigs underwent PCI procedures following approved protocols at Indiana University School of Medicine. In these studies, a 10 mm balloon catheter was positioned in various arteries with diameters approximately 3 to 4 mm in diameter. The balloon was inflated to 1 to 1.3 times the vessel diameter to effectively denude the vessel, and was immediately followed by a ClearwayRX delivery balloon sized to the vessel diameter.

Denuded arteries were treated by injecting through the delivery balloon between 2 and 10 mL of either saline control or various concentrations of peptidoglycan dissolved in 1×PBS pH 7.4. Angiography with contrast dye was recorded before and after each treatment to monitor balloon positioning and vessel diameters. After 14 days, pigs were sacrificed and vessels harvested for histological evaluation using H&E and Verhoff-Van Gieson staining. The pigs were heparinized during PCI procedures but were not on antiplatelet therapy at any time.

Denuded vessels treated with the sham control responded to balloon injury with significant vasospasm. Vasospasm is commonly observed in swine and is a direct consequence of platelet binding and activation on the denuded endothelium. Vasospasm was used as a measure of effective inhibition of platelet binding and inhibition of platelet activation with the peptidoglycan treatment. The severity of vasospasm corresponds to the degree of platelet deposition on the denuded endothelium. The degree of vasospasm was quantified by measuring the vessel diameter before and after balloon injury and treatment using ImageJ software, and percent occlusion calculated.

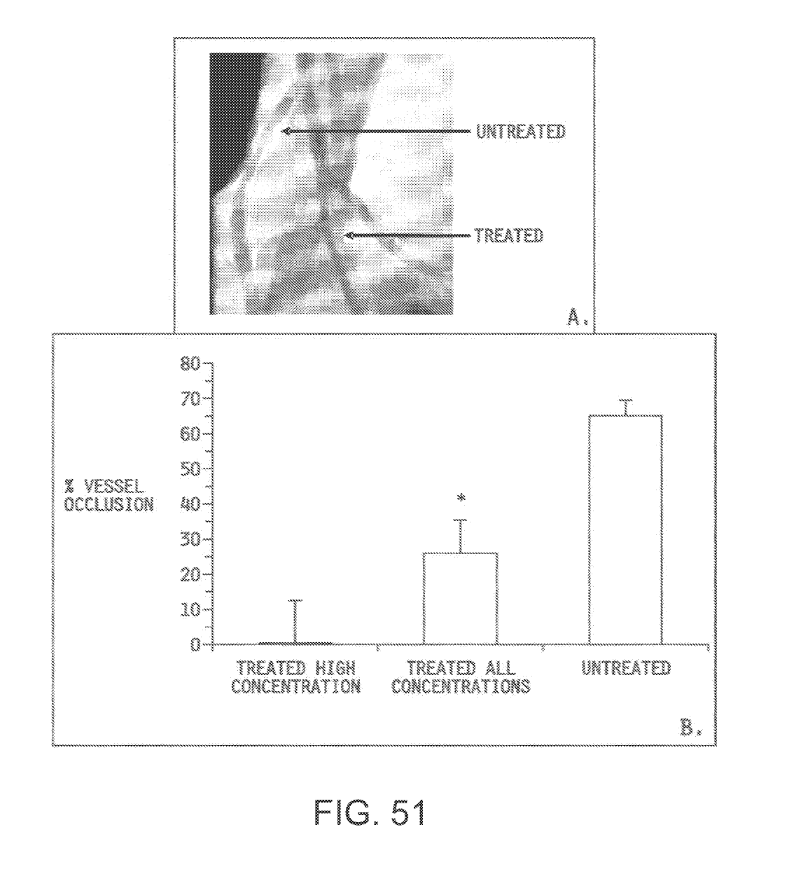

As shown in FIG. 51, the peptidoglycan treatment significantly inhibited vasospasm and platelet binding to the denuded endothelium. At higher concentrations of 2.5 mg/mL or more, vasospasm is almost completely inhibited, which corresponds to in vitro studies in which this concentration shows maximal inhibition of platelet activation.

### Example 42

### Histological Evaluation of Vessels

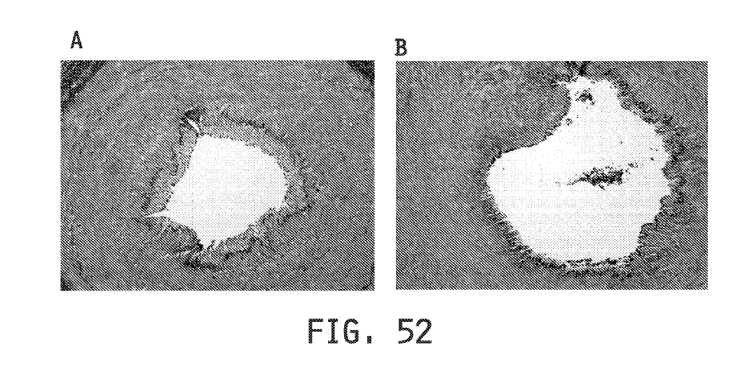

Histological evaluation was performed on balloon injured vessels 14 days post injury to assess intimal hyperplasia. No adverse responses were observed, and preliminary results at high peptidoglycan concentrations suggested that peptidoglycan inhibits intimal hyperplasia as shown in FIG. 52.

### Delivery:

For optimal delivery of the peptidoglycan treatment, application on the denuded endothelium should occur immediately following balloon injury. The peptidoglycan can prevent platelets from binding to the denuded endothelium. A system in which a single balloon capable of expanding the vessel as well as delivering the peptidoglycan treatment can be used, however effective platelet inhibition is achieved under current delivery protocols.

### Example 43

### Immunohistochemistry

Fresh carotid arteries were harvested from pigs and placed in cold 1×PBS, and tested within 5 hours of harvest. Arteries were cut open and denuded with a rubber policeman and were then cut into approximately 4 mm segments and placed into a 96-well plate. Biotin labeled peptidoglycan DS-SILY 18-biotin was incubated at 10 μM dissolved in 1×PBS pH 7.4 for 15 min at room temperature. Control arteries were incubated with 1×PBS pH 7.4. Arteries were snap frozen in liquid nitrogen, cut into 7 μm sections and air dried for 45 min, then stored at −20° C. until staining. Tissue was fixed in ice cold acetone, air dried and washed with DI water. Sections were incubated with streptavidin-HRP for 30 min, washed with DI water, incubated with DAB for 10 min, rinsed and stained with hematoxylin for 5 min. Brightfield images were taken at 10×.

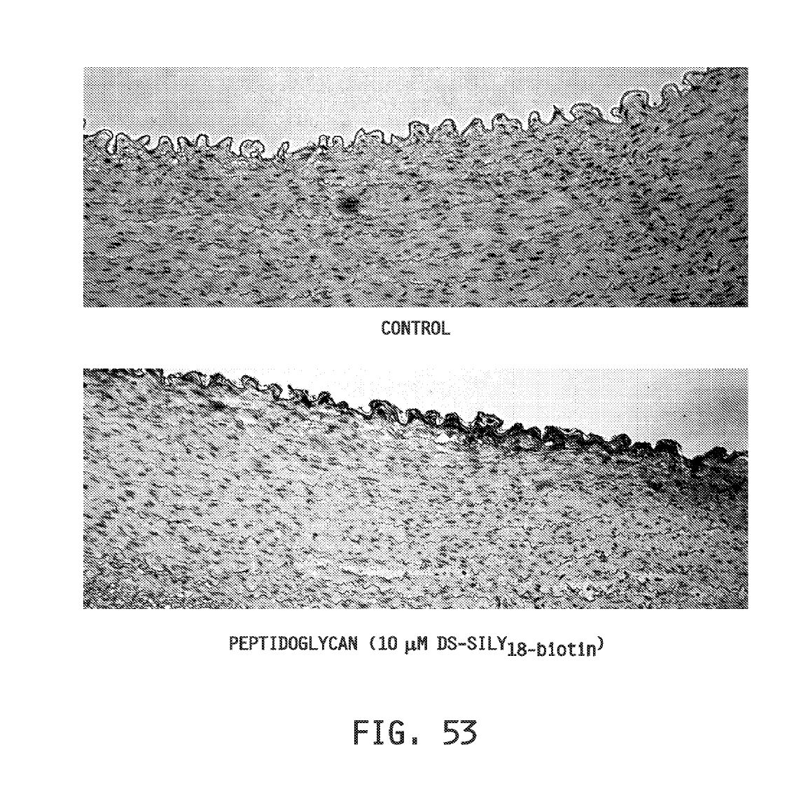

As shown in FIG. 53, denuded arteries incubated with labeled peptidoglycan stained positive at the denuded surface in contrast to control arteries which did not take up the stain. The peptidoglycan uniformly bound largely at the surface, which has a higher concentration of collagen, rather than deeper into the tissue.

### Example 44

### Inhibition of Whole Blood Binding to Collagen Under Flow

Flow kits were obtained from Ibidi (Martinsried, Germany). Each channel was coated with fibrillar collagen as described for static microplate studies. Excess collagen was removed from the flow channel by extensive rinsing with 1×PBS through the channel. DS-SILY₁₈ was incubated in the channel at a concentration of 50 μM for 15 min at 37° C., and unbound peptidoglycan was rinsed with 1×PBS. Control channels consisted of collagen not treated with peptidoglycan.

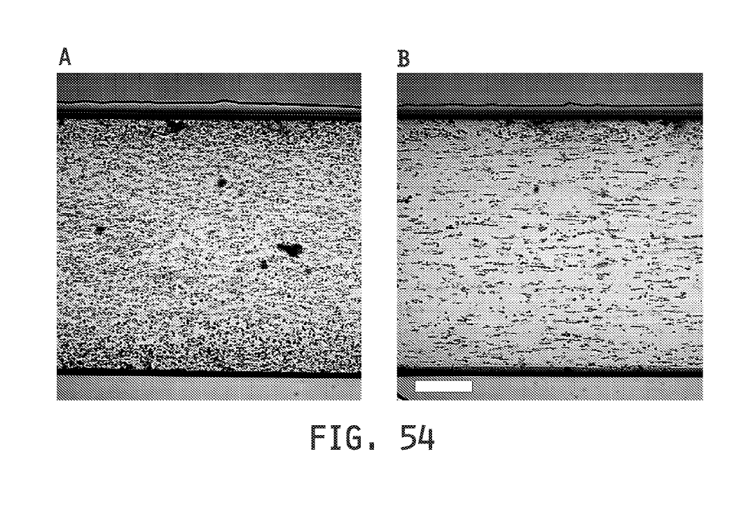

Whole blood was pushed through the flow channels by a syringe pump at a flow rate of 5.6 mL/hr, corresponding to a physiologically relevant shear rate of 1000 s⁻¹ (Badimon L, Badimon J J, Turitto V T, Vallabhajosula S, Fuster V. Platelet thrombus formation on collagen type I—a model of deep vessel injury—influence of blood rheology, von Willebrand Factor, and blood-coagulation. Circulation 1988; 78(6):1431-42). After 5 min of flow, 1×PBS pH 7.4 was pushed through at the same flow rate for 10 min to wash unbound cells. Brightfield images (n=3) were taken of each flow channel with a 10× objective. Images were thresholded and quantified for cellular coverage using ImageJ (NIH, Bethesda, Md.) and MatLab (Mathworks, Natick, Mass.) respectively. Control channels were assumed to have complete cellular coverage. The results in FIG. 54 show that there was much less blood cell binding to collagen when collagen was treated with DS-SILY₁₈.

What is claimed is:

1. A peptidoglycan comprising a glycan and from 15 to 25 synthetic peptides of up to 40 amino acids comprising the amino acid sequence GELYKSILY (SEQ ID NO: 25).

2. The peptidoglycan of claim 1, wherein the glycan is dermatan sulfate or chondroitin sulfate.

3. The peptidoglycan of claim 1, wherein the peptide is a synthetic peptide of up to 40 amino acids comprising the amino acid sequence RRANAALKAGELYKSILY (SEQ ID NO: 17).

4. The peptidoglycan of claim 1, wherein the peptidoglycan comprises about 20 synthetic peptides.

5. The peptidoglycan of claim 1, wherein the peptidoglycan comprises about 18 synthetic peptides.

6. The peptidoglycan of claim 1, wherein the peptide is bonded to the glycan via a linker.

7. A composition comprising the peptidoglycan of claim 1 and one or more additional agent.

8. The composition of claim 7, wherein each one or more additional agent is independently selected from the group consisting of a pH buffering agent, ionic strength modifying agent, viscosity modulating agent, and solubility-enhancing agent.
